(19) United States
(12) Patent Application Publication
(10) Pub. No.: US 2014/0165697 A1
(43) Pub. Date: Jun. 19, 2014

(54) MOUTHPIECE OF BREATH COMPONENT MEASURING DEVICE, BREATH COMPONENT MEASURING ASSEMBLY, BREATH COMPONENT MEASURING DEVICE, AND BREATH COMPONENT MEASURING SYSTEM

(71) Applicant: Tanita Corporation, (US)

(72) Inventors: Kei MOCHIZUKI, Saitama (JP); Kiyoshi SAGAWA, Saitama (JP)

(73) Assignee: TANITA CORPORATION, Tokyo (JP)

(21) Appl. No.: 13/842,335

(22) Filed: Mar. 15, 2013

(30) Foreign Application Priority Data
Dec. 17, 2012 (JP) 2012-274237
Dec. 17, 2012 (JP) 2012-274238
Dec. 17, 2012 (JP) 2012-274239
Dec. 17, 2012 (JP) 2012-274240

(51) Int. Cl. G01N 33/497

(52) U.S. Cl. CPC G01N 33/4972
USPC 73/23.3

(57) ABSTRACT

A mouthpiece which is attached to a breath component measuring device for measuring an alcohol concentration of breath of a subject, has: a blow port in which the breath of the subject is blown; a breath flow path which allows the breath blown in the blow port to flow in the breath component measuring device; and a sound producing portion which produces a sound by means of a flow of the breath.

### CROSS-REFERENCES TO RELATED APPLICATION

This application claims priority from Japanese Patent Application Serial Nos. 2012-274237, 2012-274238, 2012-274239, and 2012-274240, respectively filed Dec. 17, 2012, the contents of which are incorporated herein by reference in their entireties.

### TECHNICAL FIELD

The present invention relates to measurement of a gas concentration of alcohol contained in breath.

### BACKGROUND

In recent years, a mood of preventing drunken-driving is growing. Passenger vehicle transportation business operators and motor truck transportation business operators preferably check whether or not drivers are drunk by using an alcohol checker before leaving from business offices or after going back to business offices, and record the results. In Japan, the passenger vehicle transportation business operators and motor truck transportation business operators were made obligatory to record whether or not drivers are drunk. Further, even when drivers are away from business offices, preferably, whether or not the drivers are drunk is checked by using an alcohol checker, and data indicating the results is sent to the business offices.

Furthermore, an owner of an alcohol checker, that is, a device for measuring breath components (expiration components), preferably inspects the breath component measuring device at an adequate cycle, that is, for example, cyclically. In Japan, carrying valid alcohol checkers at all times was made obligatory in 2011 for passenger vehicle transportation business operators and the motor truck transportation business operators.

A device which detects an alcoholic influence of a driver based on alcohol contained in breath of the driver of a vehicle is proposed (for example, Japanese Patent Application Publication No. 2004-245800). An alcohol checker disclosed in Japanese Patent Application Publication No. 2004-245800 has an alcohol concentration sensor which measures an alcohol gas concentration in breath flowing from a blow port and a sensor unit which detects that a pressure of breath is a predetermined value or more, and prevents, for example, a trick of a sobriety test from being made by intentionally not blowing breath or blowing breath to a sensor only for a short period of time.

### SUMMARY

However, if a pressure sensor such as the alcohol checking device disclosed in Japanese Patent Application Publication No. 2004-245800 is used, since the pressure sensor is highly likely to break down when a load is large, a durable period of a product becomes short.

It is therefore an object of an embodiment of the present invention to provide a mouthpiece of a breath component measuring device, a breath component measuring assembly, a breath component measuring device and a breath component measuring system which allow subjects to continue breathing and have a longer durable period of the device.

A mouthpiece of the breath component measuring device according to an embodiment of the present invention is attached to a breath component measuring device which measures an alcohol concentration of breath of a subject, and has a blow port in which the breath of the subject is blown; a breath flow path which allows the breath blown in the blow port to flow in the breath component measuring device; and a sound producing portion which produces a sound by means of a flow of the breath.

This mouthpiece is equipped with a sound producing portion which produces a sound by a flow of breath blown by the subject. When the breath component measuring device to which this mouthpiece is attached measures the alcohol concentration of breath of the subject, a sound is produced when the subject blows breath in the mouthpiece. It is possible to check based on this sound that the subject continues breathing. Hence, even when the subject does not intentionally blow breath or blows breath in the mouthpiece only for a short period of time, the mouthpiece does not produces a sound and such cheating immediately is found out. When, for example, the alcohol concentration is measured in front of a person who monitors or supervises the subject, the subject cannot help but continuously blowing breath in the mouthpiece. A sound is used to check whether or not breathing continues, so that no pressure sensor is needed for such a breath measuring device. Although, for example, an acoustic measuring device can be used to check whether or not breathing continues, a durable period is long compared to use of a pressure sensor.

A breath component measuring assembly according to an embodiment of the present invention has the mouthpiece and the breath component measuring device to which the mouthpiece is attached, wherein the breath component measuring device has a flow path in which breath flows from the breath flow path of the mouthpiece; a gas sensor which measures an alcohol concentration of the breath in the flow path; an acoustic measuring device which measures an acoustic level of a sound produced by the mouthpiece; and a control unit which, when the acoustic level measured by the acoustic measuring device continuously exceeds a threshold for a certain period of time, starts measuring the alcohol concentration using the gas sensor.

According to an embodiment of the present invention, a mouthpiece is attached to a breath component measuring device to form a breath component measuring assembly together with a mouthpiece, and the breath component measuring device has a flow path in which breath flows from a breath flow path of the mouthpiece; a gas sensor which measures an alcohol concentration of the breath in the flow path; an acoustic measuring device which measures an acoustic level of a sound produced by the mouthpiece; and a control unit which, when the acoustic level measured by the acoustic measuring device continuously exceeds a threshold for a certain period of time, starts measuring the alcohol concentration using the gas sensor.

In the breath component measuring assembly and the breath component measuring device according to the embodiment of the present invention, when the acoustic level of the sound produced by the mouthpiece continuously exceeds the threshold for the certain period of time, the control unit starts measuring the alcohol concentration using the gas sensor. Hence, when the alcohol concentration is measured, the subject to be examined cannot help but continuously blowing breath in the mouthpiece. Although the acoustic measuring device can be used to check whether or not breathing continues, a durable period thereof is longer than that in case of use of a pressure sensor.

In the acoustic measuring device, an information processing device which can communicate with the breath component measuring device may be arranged instead of the breath component measuring device. In this case, the breath component measuring system according to an embodiment of the present invention may have a breath component measuring assembly which comprises the mouthpiece and a breath component measuring device to which the mouthpiece is attached; and an information processing device which can communicate with the breath component measuring device, wherein the breath component measuring device has: a flow path in which breath flows from the breath flow path of the mouthpiece; a gas sensor which measures an alcohol concentration of the breath in the flow path; a control unit which starts measuring an alcohol concentration using the gas sensor, the information processing device has an acoustic measuring device which measures an acoustic level of a sound produced by the mouthpiece, and the control unit of the breath component measuring device starts measuring the alcohol concentration using the gas sensor when the acoustic level measured by the acoustic measuring device continuously exceeds a threshold for a certain period of time.

### BRIEF DESCRIPTION OF THE DRAWING

Other features and advantages of the present mouthpiece of breath component measuring device, breath component measuring assembly, breath component measuring device, and breath component measuring system will be apparent from the ensuing description, taken in conjunction with the accompanying drawings, in which:

FIG. 1 is a perspective view illustrating an outlook of a breath component measuring assembly according to an embodiment of the present invention;

FIG. 2A is a front view illustrating a breath component measuring device of the breath component measuring assembly in FIG. 1;

FIG. 2B is a side view of the breath component measuring device;

FIG. 2C is a top view of the breath component measuring device;

FIG. 3A is a top view describing a mouthpiece of the breath component measuring assembly in FIG. 1;

FIG. 3B is a front view of the mouthpiece;

FIG. 3C is a side view of the mouthpiece;

FIG. 3D is a back view of the mouthpiece;

FIG. 3E is a bottom view of the mouthpiece;

FIG. 4A is a cross-sectional view along 4A-4A in FIG. 3A;

FIG. 4B is a cross-sectional view along 4B-4B in FIG. 3C;

FIG. 4C is a cross-sectional view along 4C-4C in FIG. 3C;

FIG. 5A is a front view illustrating the breath component measuring assembly in which the mouthpiece is attached to the breath component measuring device in a first direction;

FIG. 5B is a front view illustrating the breath component measuring assembly in which the mouthpiece is attached to the breath component measuring device in a second direction;

FIG. 6 is an explanatory view illustrating an internal structure of the breath component measuring device according to the present embodiment;

FIG. 7A is a block diagram illustrating an entire configuration of the breath component measuring device according to the present embodiment;

FIG. 7B is a block diagram illustrating an internal module of a control unit of the breath component measuring device;

FIG. 8A is a conceptual diagram illustrating the breath component measuring system which has a mobile computer according to the present embodiment;

FIG. 8B is a conceptual diagram illustrating a breath component measuring system which has a fixed mounted computer according to the present embodiment;

FIG. 9 is a block diagram illustrating an internal configuration of an information processing terminal according to the present embodiment;

FIG. 10 is a flowchart illustrating a breath component measuring method according to the present embodiment;

FIG. 11A is a front view illustrating the breath component measuring device of the breath component measuring assembly;

FIG. 11B is a side view of the breath component measuring device;

FIG. 11C is a top view of the breath component measuring device;

FIG. 12A is a top view describing the mouthpiece of the breath component measuring assembly;

FIG. 12B is a front view of the mouthpiece;

FIG. 12C is a side view of the mouthpiece;

FIG. 12D is a back view of the mouthpiece;

FIG. 12E is a bottom view of the mouthpiece;

FIG. 13A is a cross-sectional view of the mouthpiece in FIG. 12A along 13A-13A;

FIG. 13B is a cross-sectional view along 13B-13B in FIG. 12C;

FIG. 13C is a cross-sectional view along 13C-13C in FIG. 12C;

FIG. 14 is an explanatory view illustrating an internal configuration of the breath component measuring device in FIGS. 11A to 11C;

FIG. 15A is a block diagram illustrating an entire configuration of the breath component measuring device in FIGS. 11A to 11C;

FIG. 15B is a block diagram illustrating an internal module of a control processing unit of the breath component measuring device;

FIG. 16 is a block diagram illustrating an internal configuration of an information processing terminal used in a breath component measuring system;

FIG. 17 is a flowchart illustrating a breath component measuring method according to an EXAMPLE 1;

FIG. 18A is a perspective view illustrating a protective cover according to an EXAMPLE 2, which is diagonally viewed from a lower side thereof;

FIG. 18B is a perspective view illustrating the protective cover, which is diagonally viewed from an upper side thereof;

FIG. 19 is a side view illustrating the breath component measuring device to which the protective cover is attached;

FIG. 20 is a top view of the breath component measuring device according to the EXAMPLE 2;

FIG. 21 is a block diagram illustrating an entire configuration of the breath component measuring device according to the EXAMPLE 2;

FIG. 22 is a block diagram illustrating an internal module of a control processing unit of the breath component measuring device according to the EXAMPLE 2;

FIG. 23A is a perspective view illustrating a test attachment according to an EXAMPLE 3;

FIG. 23B is a perspective view illustrating the test attachment, which is diagonally viewed from an upper side thereof;

FIG. 24 is a side view illustrating the breath component measuring device to which the test attachment is attached;

FIG. 25 is a top view of the breath component measuring device according to the EXAMPLE 3;

FIG. 26A is a block diagram illustrating an entire configuration of the breath component measuring device according to the EXAMPLE 3;

FIG. 26B is a block diagram illustrating an internal module of a control processing unit of the breath component measuring device according to the EXAMPLE 3;

FIG. 27 is an explanatory view illustrating an internal configuration of the breath component measuring device in FIG. 1;

FIG. 28A is a block diagram illustrating an entire configuration of the breath component measuring device in FIG. 3;

FIG. 28B is a block diagram illustrating an internal module of a control unit of the breath component measuring device;

FIG. 29 is a block diagram illustrating an internal configuration of an information processing terminal according to the present embodiment;

FIG. 30 is a flowchart illustrating a breath component measuring method according to the present embodiment;

FIG. 31 is a perspective view illustrating an outlook of a breath component measuring assembly according to an embodiment of the present invention;

FIG. 32A is a front view illustrating the breath component measuring device of the breath component measuring assembly in FIG. 31;

FIG. 32B is a side view of the breath component measuring device;

FIG. 32C is a top view of the breath component measuring device;

FIG. 33A is a top view of a mouthpiece of the breath component measuring assembly in FIG. 31;

FIG. 33B is a front view of the mouthpiece;

FIG. 33C is a side view of the mouthpiece;

FIG. 33D is a back view of the mouthpiece;

FIG. 33E is a bottom view of the mouthpiece;

FIG. 34A is a cross-sectional view of the mouthpiece in FIGS. 33A to 33E along 34A-34A;

FIG. 34B is a cross-sectional view along 34B-34B in FIG. 33C;

FIG. 34C is a cross-sectional view along 34C-34C in FIG. 33C;

FIG. 35 is a front view illustrating the breath component measuring device of the breath component measuring assembly;

FIG. 36 is an explanatory view illustrating an internal configuration of the breath component measuring device in FIGS. 2A to 2C;

FIG. 37A is a block diagram illustrating an entire configuration of the breath component measuring device in FIG. 31;

FIG. 37B is a block diagram illustrating an internal module of a control unit of the breath component measuring device;

FIG. 38A is a top view illustrating a test device according to an EXAMPLE 1;

FIG. 38B is a lower surface view of the test device;

FIG. 38C is a cross-sectional view along 38C-38C in FIG. 38A;

FIG. 39 is a cross-sectional view of the test device which indicates a use state of the test device and the breath component measuring device according to the EXAMPLE 1;

FIG. 40A is a top view illustrating a test device according to an SECOND EMBODIMENT;

FIG. 40B is a front view of the test device;

FIG. 40C is a side view of the test device;

FIG. 40D is a back view of the test device;

FIG. 40E is a bottom view of the test device;

FIG. 41A is a cross-sectional view along 41A-41A in FIG. 40A;

FIG. 41B is a cross-sectional view along 41B-41B in FIG. 40B;

FIG. 41C is a cross-sectional view along 41C-41C in FIG. 40B;

FIG. 42 is a cross-sectional view of the test device which indicates a use state of the test device and the breath component measuring device according to the EXAMPLE 2;

FIG. 43A is a perspective view illustrating a test attachment according to an EXAMPLE 3;

FIG. 43B is a perspective view illustrating the test attachment, which is diagonally viewed from an upper side thereof;

FIG. 44 is a side view illustrating the breath component measuring device to which the test attachment is attached according to EXAMPLE 3;

FIG. 45A is a top view illustrating a test device of the test attachment according to the EXAMPLE 3;

FIG. 45B is a lower surface view of the test device;

FIG. 45C is a cross-sectional view along 45C-45C in FIG. 45A; and

FIG. 46 is a cross-sectional view of the test attachment which indicates a use state of the test attachment and the breath component measuring device according to the EXAMPLE 3.

### DESCRIPTION

Hereinafter, embodiments of a breath component measuring device according to an embodiment of the present invention will be described in details with reference to the drawings.

### First Embodiment

Hereinafter, a FIRST EMBODIMENT of the present invention will be described with reference to the accompanying drawings.

### Breath Component Measuring Device

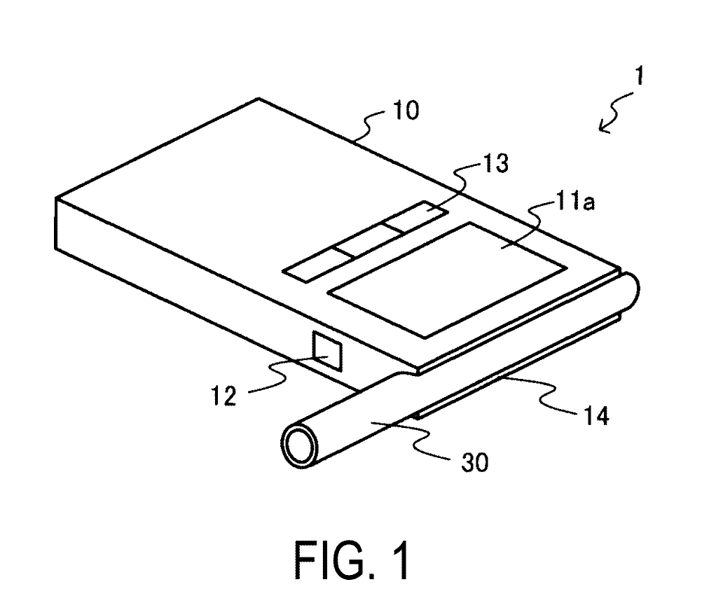

A breath component measuring assembly 1 is a device which measures an alcohol gas concentration which is a detection target gas component contained in breath of a subject, and, as illustrated in FIGS. 1, 2A-2C, 3A-3C, 4A-4C, 5A and 5B, has a breath component measuring device 10 which has a casing made of a hard material such as metal or resin and a mouthpiece 30 which is detachably attached to the breath component measuring device 10 and is made of hard material such as resin.

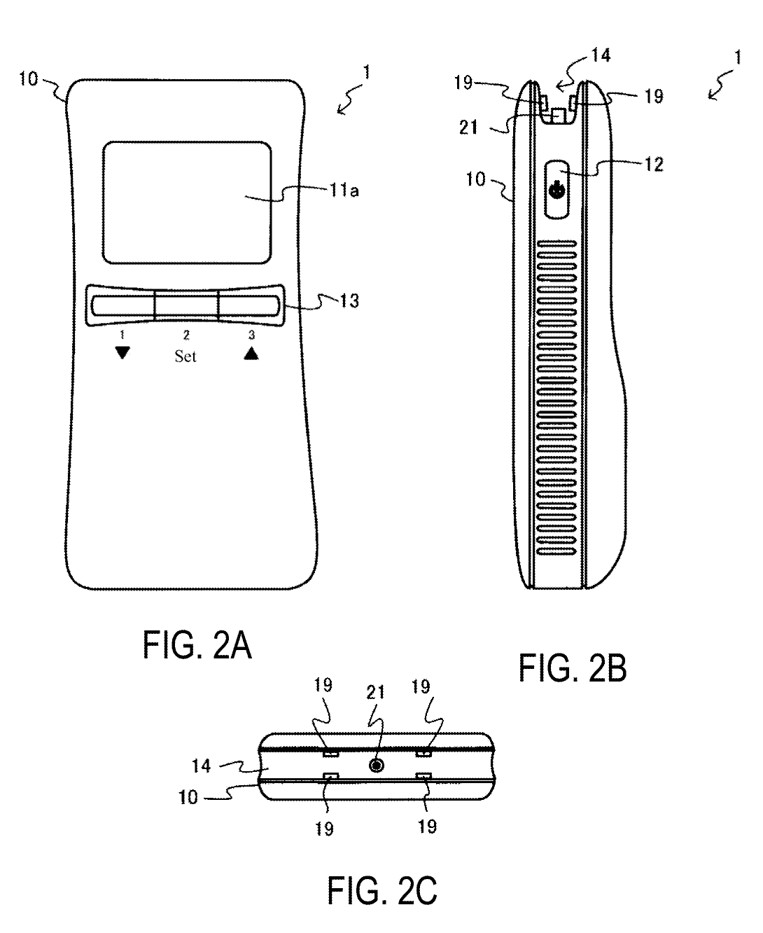

To an end portion of the breath component measuring device 10 of the breath component measuring assembly 1, the mouthpiece 30 is attached and the breath component measuring device 10 acquires and inspects breath blown in this mouthpiece 30. As illustrated in FIGS. 2A and 2B, on an outer face of the breath component measuring device 10, has a power switch 12, an input interface 13 such as operation buttons, an attachment portion 14 to which the mouthpiece 30 in which the subject blows breath is attached, a projection 21 which is provided with a guide hole for allowing breath to flow thereinside, and a display unit 11a such as an LCD (Liquid Crystal Display) which displays, for example, a measurement result of a detection target gas component contained in breath based on a detection result of a gas sensor for measuring the gas (alcohol) concentration in the breath.

Further, in the present embodiment, the breath component measuring device 10 has, for example, a communication interface which is connected with a communication cable such as the information processing terminal 7, for connecting it to an external device and a power input terminal with which a power code for supplying power from an outside is connected although not illustrated.

Furthermore, as illustrated in FIGS. 2B and 2C, engaging pieces 19 which engage with the mouthpiece 30 are provided in side surfaces of the attachment portion 14, and the breath component measuring device 10 and the mouthpiece 30 are jointed through these engaging pieces 19. The breath component measuring device 10 can have a built-in battery such as a dry cell battery thereinside as a power source, and can be carried by a user.

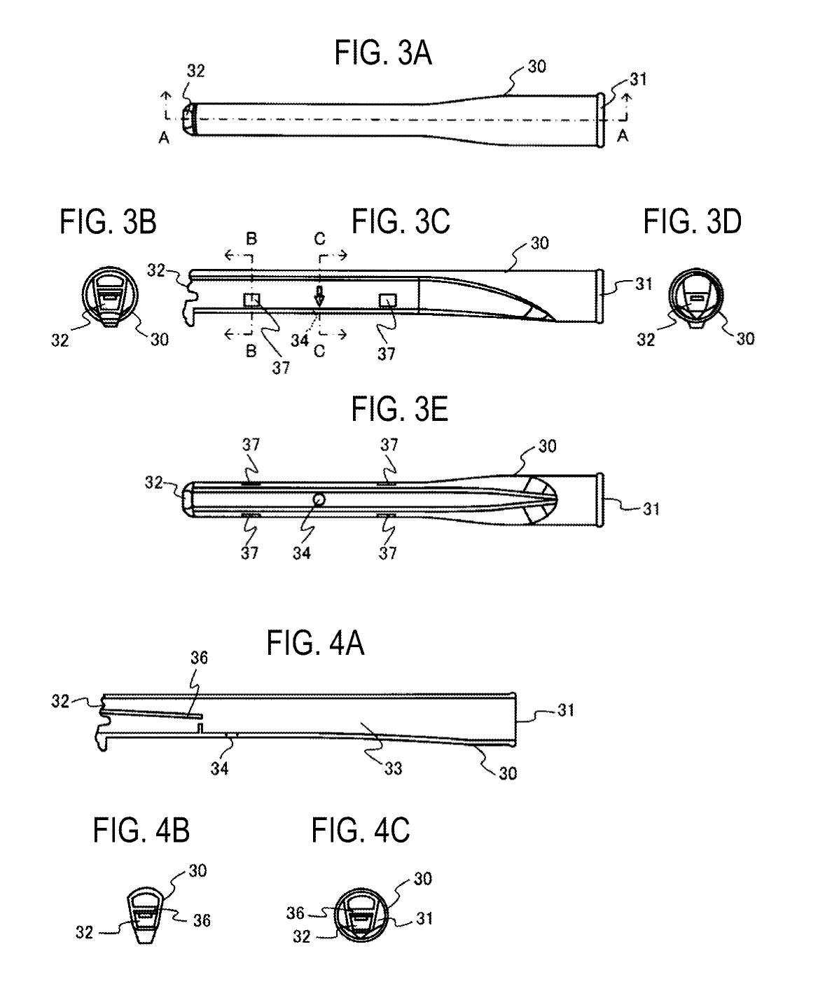

The mouthpiece 30 is a tubular blow unit in which the subject to be examined blows breath, and has a blow port 31 in which the subject blows breath, an outlet 32 which discharges breath and a breath flow path 33 which is formed between the blow port 31 and the outlet 32 and allows the breath blown from the blow port 31 to pass therethrough as illustrated in FIGS. 3A to 3C and 4A to 4C. In the breath flow path 33, a penetration hole 34 to which the projection 21 of the breath component measuring device 10 is fitted when the mouthpiece 30 is attached to the breath component measuring device 10, and a partitioning wall (sound producing portion) 36, which is formed on the outlet 32 side and separates a flow of the breath passing through the breath flow path 33 and produces a sound, are formed. The penetration hole 34 fits into the projection 21 to make the breath flow path 33 and a guide hole 21a formed in the projection 21 communicate with each other. When air flows therein from the blow port 31, the mouthpiece 30 produces a sound similar to a whistle.

Even when the subject does not intentionally blow breath or blows breath in the mouthpiece only for a short period of time, a sound is not produced by the mouthpiece 30 and such cheating immediately is found out. To measure an alcohol concentration in front of a person who monitors or supervises the subject to be examined, the subject cannot help but continually blowing breath in the mouthpiece 30.

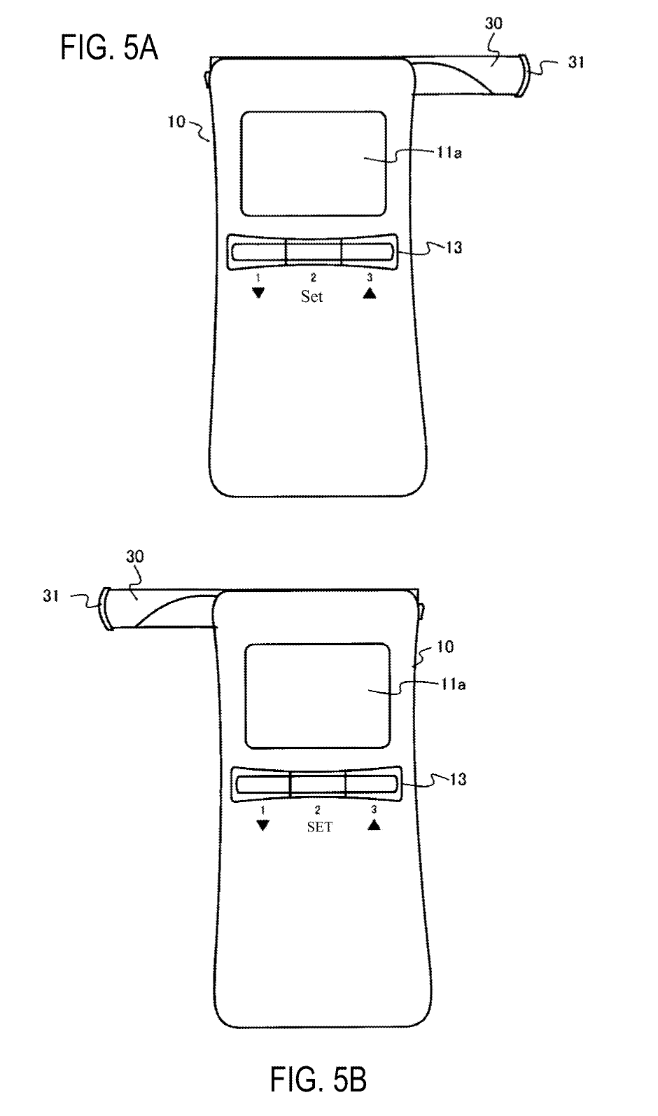

Further, on outer face of the mouthpiece 30, engaging holes 37 are formed so as to engage with the engaging pieces 19 when attached to the breath component measuring device 10. As illustrated in FIG. 5A or 5B, when the mouthpiece 30 is attached to the attachment portion 14, the engaging pieces 19 of the breath component measuring device 10 are fitted to the engaging holes 37 of the mouthpiece 30, so that the mouthpiece 30 is stably held by the attachment portion 14.

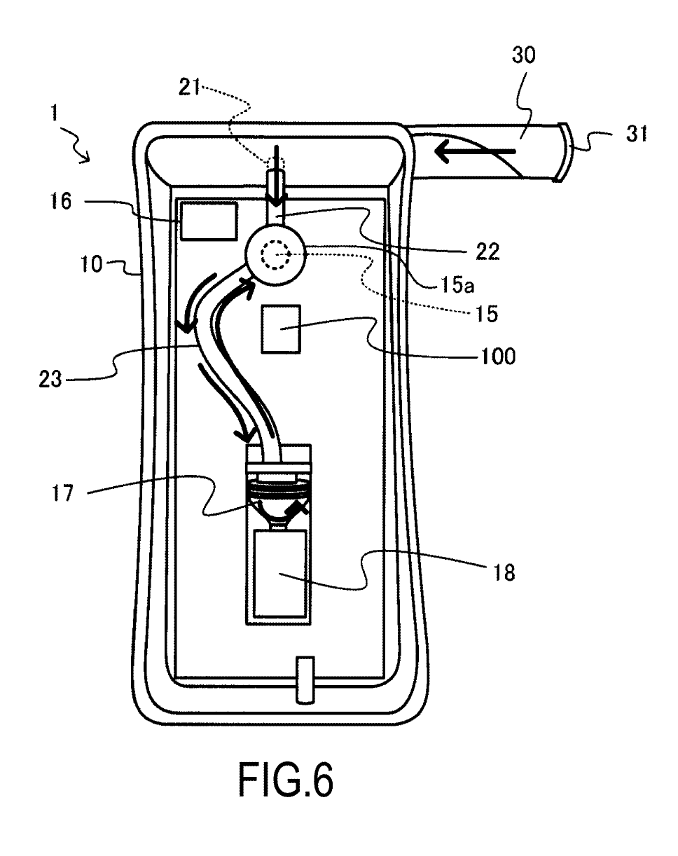

As illustrated in FIG. 6, an internal structure of this breath component measuring device 10 has a gas sensor 15 which measures the alcohol concentration of breath blown in the breath component measuring device 10, an air barrel 17 which is a container for accumulating breath thereinside and can be expanded and contracted, and a solenoid 18 which contracts the air barrel 17 and guides the breath in the air barrel 17 to the gas sensor 15. Further, a flow path of breath inside the breath component measuring device 10 includes a flow path 22 which guides breath blown from the guide hole provided in the projection 21, to a gas sensor room 15a, and a flow path 23 which connects the gas sensor room 15a to the air barrel 17. The gas sensor room 15a is also part of the flow path, and the gas sensor 15 is arranged inside the gas sensor room 15a.

The gas sensor 15 is a detecting unit which has a gas sensing body, is accommodated inside the gas sensor room 15a of the breath component measuring device 10 and detects gas in breath. At least part of breath blown in the breath flow path contacts the gas sensing body. In the present embodiment, an electrochemical sensor which has a gas sensing body in which a current flows by contact with alcohol, and detects the alcohol concentration in the gas according to a value of a flowing current, is used as the gas sensor 15. While, for example, Pt or Pt alloys are used as an anode and a cathode of this electrochemical sensor, sulfuric acid (H₂SO₄) is used for electrolyte, wherein a change in a current produced when alcohol molecules become oxidized by platinum catalyst is measured.

In addition, the gas sensor 15 only needs to detect the alcohol concentration contained in breath, and can be adopted from various alcohol sensors such as a semiconductor sensor for detecting the alcohol concentration in gas, based on electrical resistance which changes according to a reaction of oxygen adsorbed to a metal oxide and alcohol in the gas.

Near the mouthpiece 30 and inside the breath component measuring device 10, provided is an acoustic sensor 16 which is an acoustic measuring device for measuring a sound produced by the breath passing through the breath flow path 33. This acoustic sensor 16 measures an acoustic level of the sound produced by the mouthpiece 30 when the breath blown in the mouthpiece 30 passes through the breath flow path 33. For the acoustic sensor 16, for example, a microphone which receives an acoustic wave and converts the acoustic wave into an electrical signal can be used. Further, the acoustic sensor 16 may have a great sensitivity with respect to the frequency of the sound produced by the mouthpiece 30, and a less sensitivity with respect to the other frequencies. In this case, even when noise in the surrounding is significant, if the frequency of the noise is different from the frequency of the sound of the mouthpiece 30, it is possible to accurately measure the acoustic level of the sound produced by the mouthpiece.

Further, a control unit 100 such as a CPU is provided on a circuit substrate of the breath component measuring device 10, and this control unit 100 controls the above measurement of breath components, and controls the entirety of the breath component measuring device 10 such as an input of the power switch 12 and an operation button, and an output of the display unit 11a.

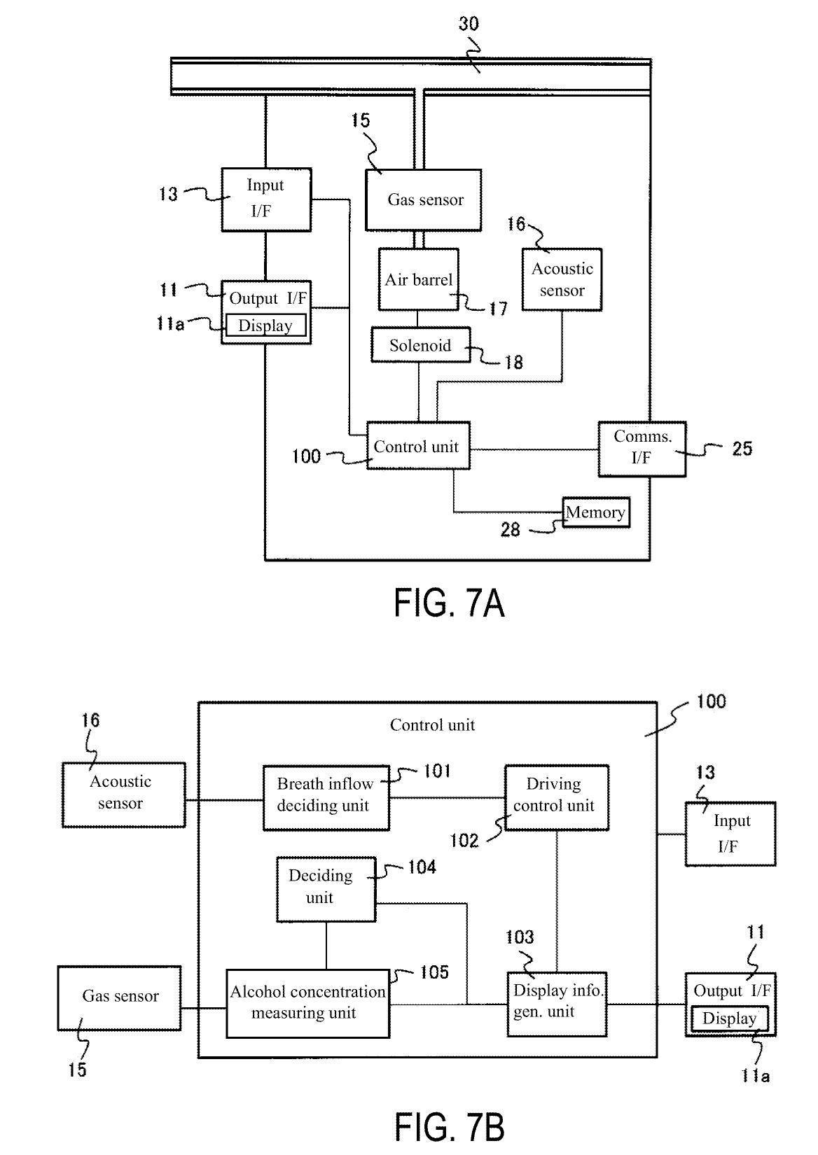

Next, a function of the breath component measuring device 10 will be described below. FIG. 7A is a block diagram illustrating an entire configuration of the breath component measuring device 10 according to the present embodiment, and FIG. 7B is a block diagram illustrating an internal module of the control unit 100. In addition, a “module”, which is used in the specification, is configured by hardware such as an apparatus or a device etc., software which has a function thereof or a combination thereof, and means a functional unit for achieving a predetermined operation.

The breath component measuring device 10 has the input interface 13, an output interface 11, a communication interface 25, a memory 28 and the control unit 100 as data processing modules which measure breath components.

The input interface 13 is a device such as an operation button, a touch panel or a jog dial which receives an input of a user operation. The output interface 11 is a device such as a display or a speaker which outputs an image or a sound. In particular, this output interface 11 includes a display unit 11a which is an LCD for displaying a measurement result of a detection target gas component contained in breath based on a detection result of the gas sensor 15, and information such as an operation guide.

The communication interface 25 is a communication interface with which the communication cable is connected, and transmits, for example, a measurement result to the information processing terminal connected through the communication cable. Instead of the communication cable, wireless communication such as Bluetooth (registered trademark) may be used.

The memory 28 is a memory device such as ROM or RAM which stores various items of data, and stores a threshold value of the acoustic sensor 16 which determines whether or not measurement can be performed, a threshold of an alcohol concentration value which determines whether or not an operation is allowed, and information such as a measurement result.

The control unit 100 is a computation processing device such as a CPU, and is a module which virtually configures each functional module by executing various programs on this control unit 100. In the present embodiment, as illustrated in FIG. 7B, the control unit 100 has a driving control unit 102, a display information generating unit 103, a deciding unit 104, a breath inflow deciding unit 101 and an alcohol concentration measuring unit 105.

The driving control unit 102 is a module which drives each device of the breath component measuring device 10, and drives, for example, the acoustic sensor 16, the gas sensor 15 and the display unit 11a. Particularly, the driving control unit 102 drives the solenoid 18, when acquiring from the breath inflow deciding unit 101a signal indicating that blow of breath is completed, so as to send breath in the air barrel 17 to the gas sensor 15 and drives the gas sensor 15 to detect the alcohol concentration.

The breath inflow deciding unit 101 is a module which judges start of an inflow of breath and continuation of the breath based on a detection result of the acoustic sensor 16. More specifically, when an acoustic level of a sound produced by the mouthpiece 30 reaches a predetermined threshold or more, the breath inflow deciding unit 101 continuously detects the acoustic level in a predetermined period of time while counting this predetermined period of time (for example, about five seconds). Further, when the acoustic level detected by the breath inflow deciding unit 101 continuously exceeds the predetermined threshold, the driving control unit 102 drives the solenoid 18, and pushes the breath in the air barrel 17 back to the gas sensor room 15a. This is because, if the breath which exceeds a certain pressure continues, the amount of air required to measure the alcohol concentration can be obtained in the air barrel 17. Meanwhile, even when blow is started, if the acoustic level goes below the threshold during the predetermined period of time, the breath inflow deciding unit 101 executes error processing to perform error display on the screen of the display unit 11a thereby displaying an instruction of redoing of blow. The subject needs to take a deep breath and exhale breath for a long period of time to obtain an alcohol measurement result.

The alcohol concentration measuring unit 105 is a module for measuring the alcohol concentration, which is the detection target gas component contained in breath, based on a detection result of the gas sensor 15, and, more specifically, calculates the alcohol concentration in the breath based on a detection result detected when air in the air barrel 17 is sent out to the gas sensor 15 by the driving control unit 102. Thus, the alcohol concentration measuring unit 105 starts measuring the alcohol concentration using the gas sensor 15 when the acoustic level measured by the acoustic sensor 16 continuously exceeds the threshold for a certain period of time, and does not start measuring the alcohol concentration using the gas sensor 15 in other cases. “Starting measuring the alcohol concentration” referred hereto means acquiring an output of the gas sensor 15 and starting calculating the alcohol concentration. Meanwhile, the alcohol concentration measuring unit 105 may drive the gas sensor 15 when the acoustic level measured by the acoustic sensor 16 continuously exceeds the threshold for a certain period of time, and may not drive the gas sensor 15 in other cases. In this case, “starting measuring the alcohol concentration” means starting an output from the gas sensor 15.

The deciding unit 104 is a module for determining whether or not driving is allowable, based on the alcohol concentration of the detection target gas component calculated by the alcohol concentration measuring unit 105. More specifically, the deciding unit 104 compares the calculated alcohol concentration in breath and the threshold stored in the memory 28, and decides that driving is allowable when the calculated alcohol concentration is the threshold or less.

The display information generating unit 103 is a module for displaying various pieces of information on the display unit 11a, and, when, for example, the power switch 12 acquires an inputted signal, it displays information about an operation guide such as an instruction to start blowing breath or a message to end blowing or displays, for example, the alcohol concentration contained in the breath detected by the gas sensor 15 or a decision result of the deciding unit 104 on the display unit 11a.

### Breath Component Measuring System

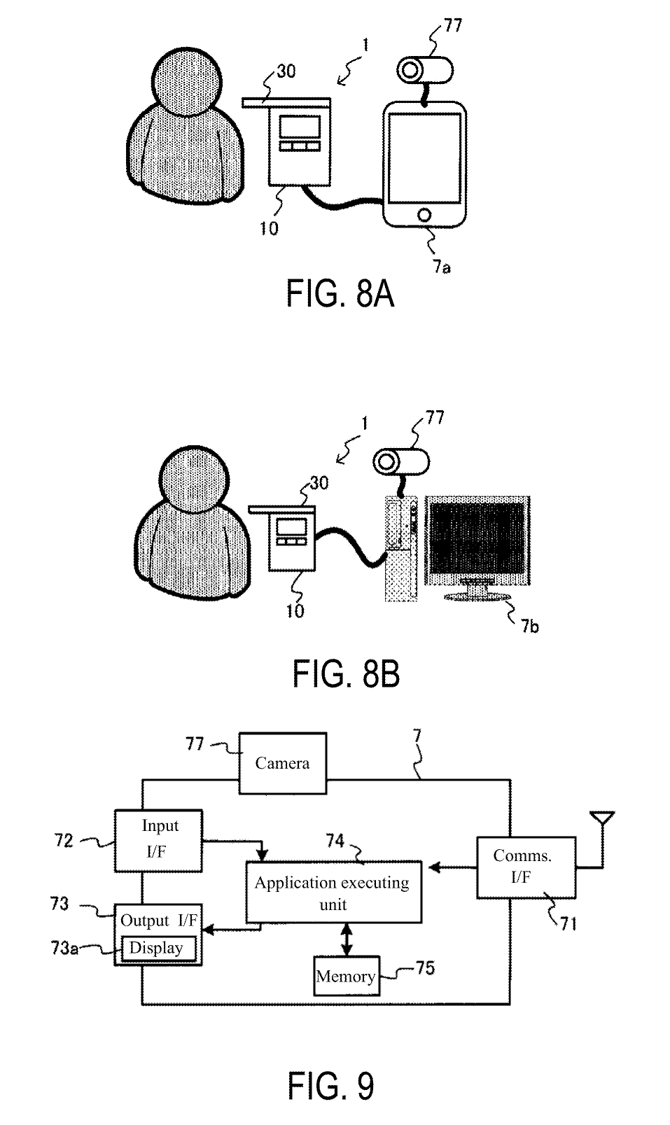

Next, the breath component measuring system which uses the above-described breath component measuring device 10 will be described below. FIGS. 8A and 8B are conceptual diagrams illustrating the breath component measuring system according to the present embodiment, and FIG. 9 is a block diagram illustrating an internal configuration of the information processing terminal 7 according to the present embodiment.

As illustrated in FIGS. 8A and 8B, the breath component measuring system according to an embodiment of the present invention has the breath component measuring assembly 1 and the information processing terminal (computer) 7 which is electrically connected to the breath component measuring device 10 of the breath component measuring assembly 1 through a communication cable, and sends, for example, information about a measurement result measured by the breath component measuring assembly 1, to the information processing terminal 7. Instead of the communication cable, wireless communication such as Bluetooth (registered trademark) may be used.

The information processing terminal 7 (7a and 7b) is a computer which has a computation processing function of the CPU and a communication processing function of the communication interface, and can be realized by a general-purpose computer such as a personal computer, a tablet PC or a dedicated device, which has a specialized function. The information processing terminal 7 may be a mobile computer, a PDA (Personal Digital Assistance), a smartphone or a mobile telephone. Further, in this information processing terminal 7 (7a and 7b), functions such as a communication function, a digital camera function, a function of executing application software and a GPS function are implemented. The information processing terminal 7 may be a mobile terminal 7a which is owned by the subject who is, for example, a driver of a vehicle as illustrated in FIG. 8A or a fixed mounted terminal 7b which is installed a business office of a passenger vehicle transportation business operator or a motor truck transportation business operator as illustrated in FIG. 8B. The mobile terminal 7a stores a mobile terminal application (measurement result processing program) and executes this application, and the fixed mounted terminal 7b stores a fixed mounted terminal application (measurement result processing program) and executes this application. The mobile terminal 7a transmits a measurement result, information which indicates the subject to be examined and position information about the terminal 7a, to a computer installed at the business office of the business operator through a mobile telephone communication network according to the mobile terminal measurement processing program. Meanwhile, the fixed mounted terminal 7b stores information which indicates the subject and a measurement result according to the fixed mounted terminal measurement result processing program.

Next, an internal configuration of the information processing terminal 7 will be described. FIG. 9 is a block diagram illustrating an internal configuration of the information processing terminal 7. As illustrated in the figure, the information processing terminal 7 has an input interface 72 and an output interface 73 as modules of user interfaces. The input interface 72 is a device such as an operation button, a touch panel or a jog dial which receives an input of a user operation. The output interface 73 is a device such as a display or a speaker which outputs an image or a sound. This output interface 73 in particular includes a display unit 73a such as a liquid crystal display.

Further, the information processing terminal 7 has a communication interface 71 as a communication module. The communication interface 71 is a module which transmits and receives various items of data through the communication network such as a mobile telephone communication network or an IP network by way of wireless communication or wired communication. Further, the information processing terminal 7 has a memory 75 and a digital camera 77. The memory 75 is a memory device which stores various items of data, and, in this memory 75, the measurement result processing program and, in addition, personal information such as a name of the subject to be examined, a telephone number thereof and a vehicle number of use are associated with one another and stored therein. The personal information about the subject is associated with identification information which is used to identify the subject, and the subject may be recognized when, for example, the subject inputs an ID and a password upon measurement or a recording medium in which identification information such as an IC card is recorded is read. The digital camera 77 is an image capturing unit which captures an image of the subject, and captures a still image of the subject under control of an application executing unit 74.

Further, the information processing terminal 7 has the application executing unit 74 as a module which executes an application. The application executing unit 74 is a common OS or a module which executes an application, and is realized by, for example, the CPU. In the present embodiment, when a measurement result processing program is installed, the application executing unit 74 can execute the measurement result processing program. The mobile terminal 7a transmits a measurement result, identification information which is used to specify the subject to be examined, and position information about the terminal 7a, to a computer installed at a business office of a business operator through a mobile telephone communication network according to the mobile terminal measurement processing program. Meanwhile, the fixed mounted terminal 7b stores in the memory 75 the measurement result and the identification information which is used to specify the subject according to the fixed mounted terminal measurement result processing program.

A signal transmitted from the breath component measuring device 10 triggers the application executing unit 74 to capture, using the digital camera 77, images of the face of the subject before and after the subject blows breath. The application executing unit 74 compares image data obtained by capturing the images of the face(s) of the user(s) before and after breath is blown, and determines whether or not the captured user images match with each other. In this processing, face detection processing of determining a face area from an image and face feature point detection processing of calculating feature point positions of the face such as the eyes, the nose and the corners of the lips are performed to compare the image data before and after breath is blown, to decide whether or not the captured user images match with each other, and to output the degree of match of both images which is a comparison result. The degree of match of both of these images may be outputted by producing a warning sound, displaying a message on a display screen and, in addition, sending a message to a predetermined electronic mail address(s) when the users are different from each other. In addition, the application executing unit 74 also has a function of displaying a profile for aligning the position of the face of the subject on the screen of the display unit 73a upon a first image capturing operation and a second image capturing operation, and making an instruction to align the face to this profile. The mobile terminal 7a sends the image data to the computer installed at the business office of the business operator. The fixed mounted terminal 7b stores the image data in the memory 75.

### Breath Component Measuring Method

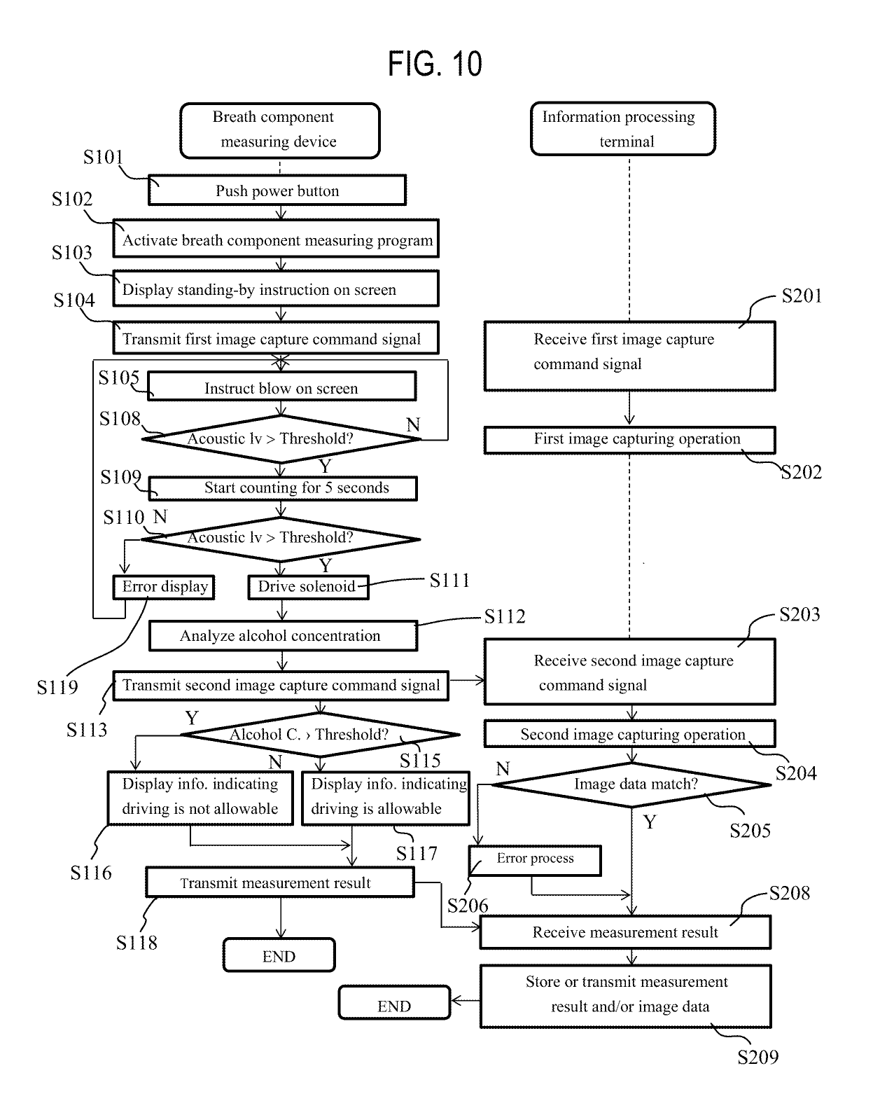

By operating the above-described breath component measuring system, it is possible to implement the breath component measuring method according to an embodiment of the present invention. FIG. 10 is a flowchart illustrating the breath component measuring method according to the present embodiment.

As illustrated in FIG. 10, first, when the subject to be examined pushes the power switch 12 (S101), the control unit 100 activates the breath component measuring program (S102). In this case, the display information generating unit 103 displays a movie of a countdown and an instruction of standing-by for a predetermined period (for example, about five seconds) on the display unit 11a until it becomes ready to perform measurement (S103).

The operation control unit 102 transmits a first image capture command signal to the information processing terminal 7 (S104). When the information processing terminal 7 receives the first image capture signal (S201), in the standing-by period (for example, five seconds) for program start-up, the application executing unit 74 executes a first image capturing operation of capturing an image of the face of the subject using the digital camera 77 (S202). Subsequently, after the standing-by period ends, the display information generating unit 103 of the breath component measuring device 10 displays an instruction to blow breath in the mouthpiece 30 for the predetermined period of time (five seconds) on the screen of the display unit 11a (S105).

Further, when the subject blows breath using the mouthpiece 30, the breath blown from the blow port 31 passes through the breath flow path 33, and the breath flows in the breath component measuring device 10 through the guide hole formed in the projection 21. The breath flows in the breath component measuring device 10, and the mouthpiece 30 produces a sound.

The breath inflow deciding unit 101 calculates an acoustic level based on the detection result of the acoustic sensor 16, and monitors the acoustic level. The breath inflow deciding unit 101 first judges whether or not the acoustic level detected by the acoustic sensor 16 exceeds the predetermined threshold (S108). Meanwhile, when the acoustic level does not exceed the threshold (“N” in S108), the breath inflow deciding unit 101 stands by until the acoustic level exceeds the threshold.

Meanwhile, when the acoustic level exceeds the threshold (“Y” in S108), while counting the predetermined period of time (for example, five seconds) from start of measurement (S109), the breath inflow deciding unit 101 judges whether or not the acoustic level detected by the acoustic sensor 16 continuously exceeds the predetermined threshold in this predetermined period of time (S110).

When the acoustic level does not continuously exceed the threshold, the breath inflow deciding unit 101 decides that the alcohol concentration cannot be measured (“N” in S110). This information is inputted to the display information generating unit 103, and the display information generating unit 103 displays error display and an instruction of redoing of blow, on the screen of the display unit 11a (S119).

Meanwhile, when the acoustic level detected by the acoustic sensor 16 continuously exceeds the predetermined threshold (“Y” in S110), the operation control unit 102 drives the solenoid 18 and sends the air in the air barrel 17 to a gas sensor 15 side (S111).

After a pressure exceeding the threshold is continuously measured, by returning the breath in the air barrel 17 to the gas sensor 15, it is possible to acquire from the subject the amount of breath which is sufficient to measure alcohol using the gas sensor. The subject needs to take a deep breath and exhale breath for a long period of time to obtain an alcohol measurement result. Even when the subject tries to supply a little amount of breath to the breath component measuring device 10 and obtain a measurement result indicating a low alcohol concentration, redoing of blow is instructed.

The alcohol concentration measuring unit 105 measures (analyzes) the alcohol concentration which is a detection target gas component contained in the breath, based on the detection result of the gas sensor 15 (S113). In this case, the operation control unit 102 controls the output interface 11, and instructs the subject to end blowing breath. Further, the operation control unit 102 transmits a second image capture command signal to the information processing terminal 7 (S113). When the information processing terminal 7 receives the second image capture command signal (S203), the application executing unit 74 executes a second image capturing operation of capturing an image of the subject using the digital camera 77 after measurement of the alcohol concentration ends (S204).

Subsequently, the image comparing unit 74a compares image data (that is, first still image data and second still image data) of the subjects the images of which are captured by the first image capturing operation and the second image capturing operation, and decides whether or not the users of both items of image data match with each other according to face recognition processing (S205). When the users of the image data do not match (“N” in S205), error processing is executed (S206). In the error processing, the image comparing unit 74a generates data which indicates that the users of both images do not match.

In the breath component measuring device 10, when the alcohol concentration measuring unit 105 finishes measuring (analyzing) the alcohol concentration of the detection target gas component, information about the alcohol concentration which is the measurement result is inputted to the deciding unit 104, and the deciding unit 104 compares the calculated alcohol concentration and the predetermined threshold with each other (S115). When the alcohol concentration in breath exceeds the threshold (“Y” in S115), the deciding unit 104 decides that driving is not allowable, and the display information generating unit 103 displays that driving is not allowable, on the screen of the display unit 11a (S116). In this case, the alcohol concentration measurement value may be displayed on the screen of the display unit 11a. Meanwhile, when the alcohol concentration is the threshold or less (“N” in S115), the deciding unit 104 decides that driving is allowable, and the display information generating unit 103 displays that driving is allowable (S117). In this case, the alcohol concentration measurement value may be displayed on the screen of the display unit 11a.

Subsequently, the breath component measuring device 10 transmits information about the measurement result to the information processing terminal 7 through the communication interface 25 (S118). When the information processing terminal 7 receives the measurement result information (S208), the measurement result and image data captured twice are stored or sent (S209). More specifically, when the information processing terminal 7 is a fixed mounted terminal 7b, the application executing unit 74 of the fixed mounted terminal 7b stores the measurement result, the image data, the measurement date data and data which indicates a name of a subject in the memory 75. When the error processing in step S206 is performed (when the user images which are captured twice, do not match with each other), the application executing unit 74 of the fixed mounted terminal 7b stores data which indicates that the users do not match with each other, in the memory 75. The fixed mounted terminal 7b may display measurement result information on the screen of the display unit 73a. When the information processing terminal 7 is the mobile terminal 7a, the application executing unit 74 of the mobile terminal 7a transmits a signal indicating the measurement result, the image data, the measurement date data and data which indicates the name of the subject, by wireless communication, using the communication interface 71 to the computer installed at a business office of a business operator. When the error processing in step S206 is performed (when user images, which are captured twice, do not match with each other), the application executing unit 74 of the mobile terminal 7a sends a report indicating data which indicates that the users do not match with each other to the computer installed at the business office of the business operator. In the present embodiment, when the user images which are obtained by the first image capturing operation and the second image capturing operation do not match with each other, data which indicates that the users in both images do not match with each other is stored in the fixed mounted terminal 7b at the business office of the business operator. Alternatively, the report indicating that the users in both images do no match with each other is sent to the business office of the business operator. Staff at a business office of a business operator learns that a subject was switched with another before and after breath is blown. That is, the staff at the business office of the business operator can learn the existence of an untrustworthy subject who did cheating.

Although the breath component measuring device 10 decides whether or not driving is allowable and the breath component measuring device 10 displays, for example, a result in the present embodiment, the present invention is not limited to this, and may transmit an alcohol concentration measurement result to the information processing terminal 7, have the information processing terminal 7 side decide whether or not driving is allowable and have the display unit 73a of the information processing terminal 7 side display the decision result as to whether or not driving is allowable.

When users in images obtained by the first image capturing operation and the second image capturing operation do not match, the information processing terminal 7 may transmit an error signal to the breath component measuring device 10, and, in the breath component measuring device 10 which receives this error signal, the control unit 100 may have the display unit 11a display a warning and the display unit 11a may instruct a subject to blow breath again and do alcohol measurement over. That is, the information processing terminal 7 may transmit to the breath component measuring device 10 an error signal which encourages the subject to try alcohol measurement again using the breath component measuring device 10. Further, the breath component measuring device 10 which receives this error signal may produce a warning sound. Even when measurement is done over again, the information processing terminal 7 may store or send data which indicates that users previously did not match.

Further, although the digital camera 77 of the information processing terminal 7 captures an image of the face of the subject in the above embodiment, for example, the breath component measuring device 10 may have a camera and the breath component measuring device 10 may capture an image of the face of a subject.

As described above, according to the embodiment, a sound is produced when the subject to be examined blows breath in the mouthpiece. It is possible to check based on this sound that the subject continues breathing. Hence, even when the subject does not intentionally blow breath or blows breath in the mouthpiece only for a short period of time, the mouthpiece does not produces a sound and such cheating immediately is found out. When, for example, the alcohol concentration is measured in front of a person who monitors or supervises the subject, the subject cannot help but continuously blowing breath in the mouthpiece. A sound is used to check whether or not breathing continues, so that the breath measuring device does not have a pressure sensor. Although the acoustic sensor 16 is used to check whether or not breathing continues in the embodiment, when an alcohol concentration is measured in front of a person who monitors or supervises a subject, the acoustic sensor 16 does not necessarily need to be used. Further, although, when measurement is performed before the eyes of a monitoring or supervising person without using an acoustic sensor, a sound produced by the mouthpiece is preferably an audible sound, when the acoustic sensor is used, the sound is not limited to an audible sound and the sound in a range which the acoustic sensor can sense may be produced.

In the breath component measuring device 10 according to the embodiment, when an acoustic level of a sound produced by the mouthpiece continuously exceeds a threshold for a certain period of time, the control unit starts measuring the alcohol concentration using the gas sensor. Hence, when the alcohol concentration is measured, the subject cannot help but continuously blowing breath in the mouthpiece. Although the acoustic sensor 16 is used to check whether or not breathing continues, a durable period is long compared to use of a pressure sensor.

Although the acoustic sensor 16 is mounted on the breath component measuring device 10 in the above embodiment, the present invention is not limited to this, and the acoustic sensor 16 may be mounted in the information processing terminal 7 which is an information processing device which can communicate with the breath component measuring device 10. Next, a modified example of an information processing terminal 7 on which an acoustic sensor is mounted will be described.

In this modified example, an application executing unit 74 calculates an acoustic level based on the detection result of an acoustic sensor, and monitors an acoustic level. The application executing unit 74 decides start of an inflow of breath and continuation of breath based on a detection result of the acoustic sensor. When the acoustic level of a sound produced by a mouthpiece 30 is a predetermined threshold or more, the application executing unit 74 continuously detects the acoustic level in a predetermined period of time while counting this predetermined period of time (for example, about five seconds).

When the acoustic level equal to or more than the threshold does not continue for the predetermined period of time (for example, five seconds) or more, the application executing unit 74 decides that blow is insufficient to detect an alcohol concentration in breath, and has a screen of a display unit 73a display that measurement is not possible and information to instruct that a subject to be examined blows breath again. Substituting this, or in addition to this, when the application executing unit 74 sends an error signal to the breath component measuring device 10 and the breath component measuring device 10 receives the error signal, the control unit 100 has a display unit 11 display a warning and has the display unit 11 instruct the subject to blow breath again to do alcohol measurement over. That is, the information processing terminal 7 may transmit to the breath component measuring device 10 an error signal which encourages the subject to try alcohol measurement again using the breath component measuring device 10. Meanwhile, when the acoustic level equal to or more than the threshold continues for a predetermined period of time (for example, five seconds), the application executing unit 74 sends a signal which drives a solenoid to the breath component measuring device 10. When acquiring this signal, the driving control unit 102 of the breath component measuring device 10 drives the solenoid 18, pushes the air (breath) in the air barrel 17 back to the gas sensor 15 and drives the gas sensor 15 to detect an alcohol concentration.

### Second Embodiment

Upon measurement of the amount of alcohol, when a device which reads identification information from an ID card is used, cost is required to dispose the device. Further, the reading device occupies a certain space. Furthermore, a person requires care to input an ID number and a password. Hence, in a SECOND EMBODIMENT, provided are a breath component measuring assembly, a breath component measuring device and a mouthpiece, which do not require care to input information for specifying a subject to be examined and do not require a special device for inputting this information.

The SECOND EMBODIMENT of the present invention will be described below with reference to the accompanying drawings. In addition, a basic configuration of a breath component measuring system (including an information processing terminal 7) according to the SECOND EMBODIMENT is the same as that of a breath component measuring system according to the FIRST EMBODIMENT illustrated in FIGS. 8A and 8B, and will not be described. In addition, in the SECOND EMBODIMENT, the same reference numerals will be assigned to the same configurations as those of the FIRST EMBODIMENT, and configurations different from the FIRST EMBODIMENT will be mainly described below.

### Example 1

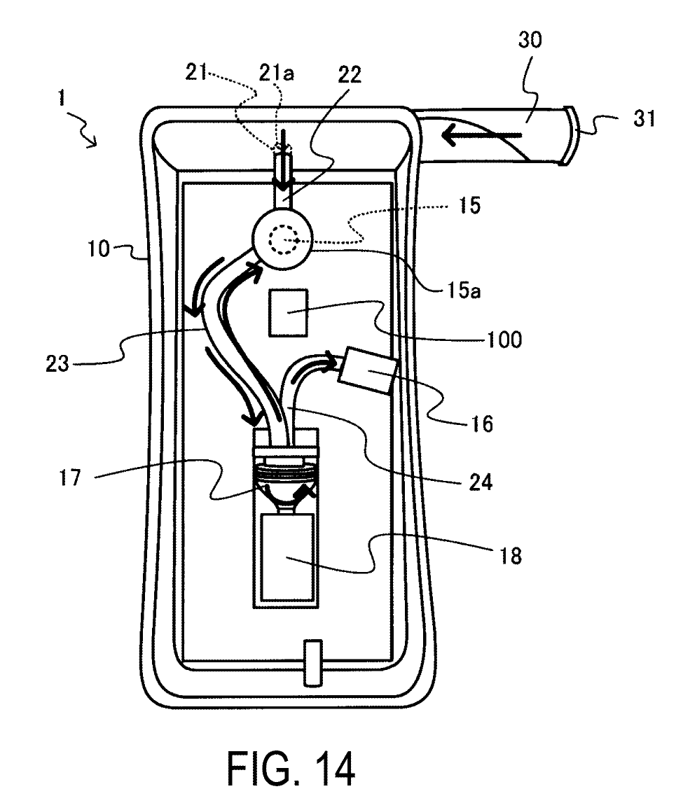

As illustrated in FIGS. 13A to 13C, inside a mouthpiece 30, a memory device 40, which can store owner information indicating an owner of the mouthpiece 30, is arranged. The memory device 40 is, for example, an IC chip, and can permanently store owner information which indicates an owner. The owner information is, for example, an owner ID. Further, the owner information may include a name of the owner and a vehicle number which the owner uses.

Furthermore, in a lower surface of the mouthpiece 30, a plurality of mouthpiece electrodes 41 and one mouthpiece contact point 42 are arranged. These are formed using conductive materials. The mouthpiece electrode 41 is electrically connected to the memory device 40, and a wiring is not illustrated. The mouthpiece electrode 41 is provided to allow a breath component measuring device 10 to read owner information stored in the memory device 40. The number of the mouthpiece electrodes 41 is not limited thereto. The mouthpiece contact point 42 is provided to allow the breath component measuring device 10 to recognize the direction of the mouthpiece 30 with respect to the breath component measuring device 10.

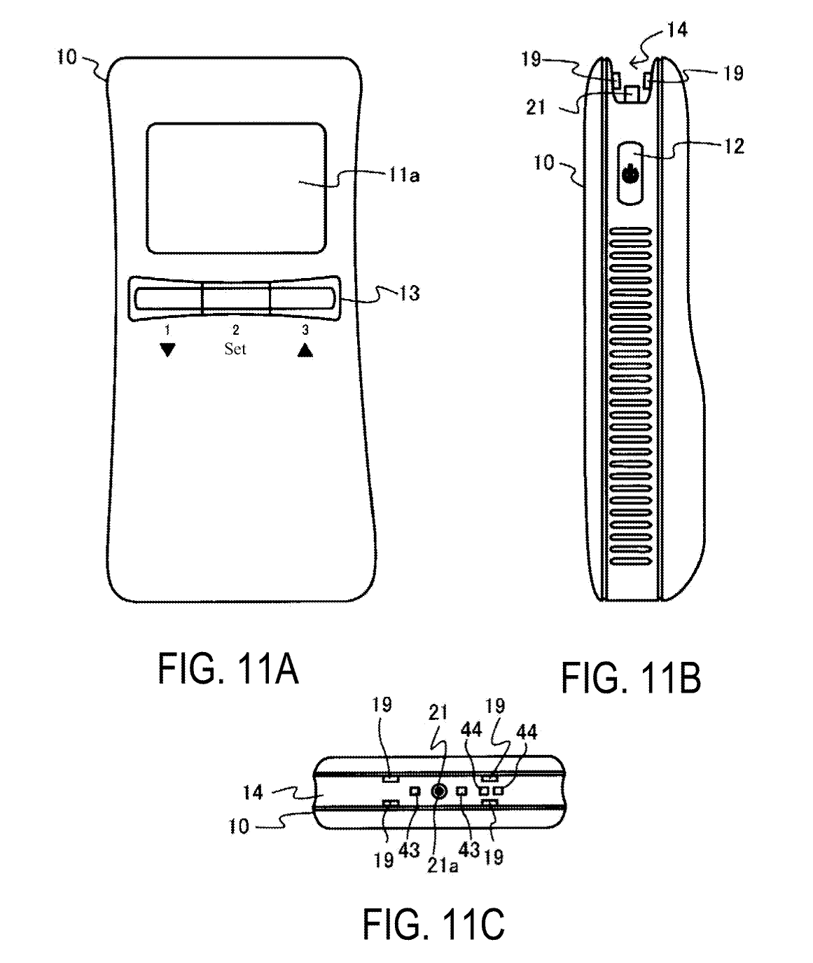

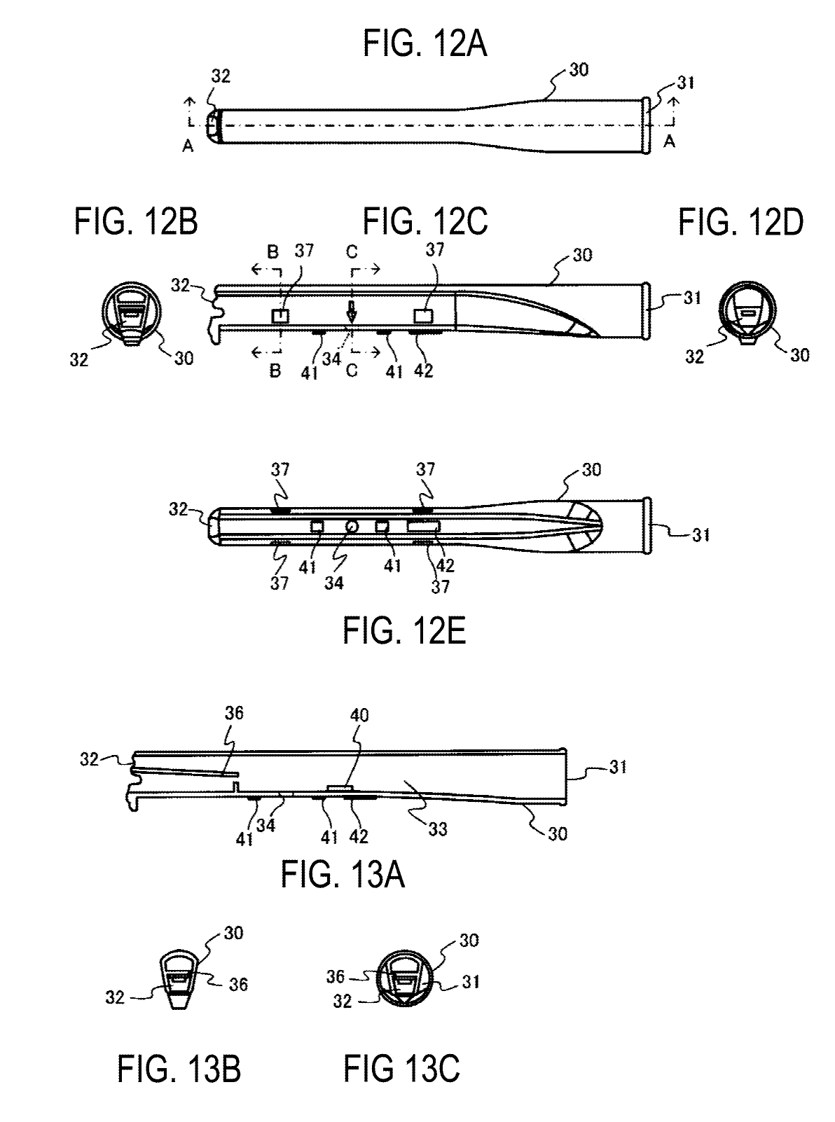

As illustrated in FIG. 11C, a plurality of owner information reading electrodes 43 and two mouthpiece direction recognizing contact points 44 are arranged in an attachment portion 14 of the breath component measuring device 10. They are made from conductive materials. The owner information reading electrodes 43 are provided to allow the breath component measuring device 10 to read owner information stored in the memory device 40. When the mouthpiece 30 is attached to the attachment portion 14, the mouthpiece electrodes 41 provided in the mouthpiece 30 is brought into contact with the owner information reading electrodes 43, thereby allowing the breath component measuring device 10 to read owner information from the memory device 40 of the mouthpiece 30. The number of owner information reading electrodes 43 is not limited thereto. The two mouthpiece direction recognizing contact points 44 are provided to allow the breath component measuring device 10 to recognize the direction of the mouthpiece 30 with respect to the breath component measuring device 10.

As shown in FIGS. 5A and 5B of the FIRST EMBODIMENT, the mouthpiece 30 can also be attached, in different directions, to the breath component measuring device 10 in the SECOND EMBODIMENT. The direction of the mouthpiece 30 at which a blow port 31 illustrated in FIG. 5A is arranged on a right side of the breath component measuring device 10 is referred to as a “first direction”, and the direction of the mouthpiece 30 at which the blow port 31 illustrated in FIG. 5B is arranged on a left side of the breath component measuring device 10 is referred to as a “second direction”. In either the first direction or the second direction of the mouthpiece 30, when the mouthpiece 30 is attached to the attachment portion 14, engaging pieces 19 of the breath component measuring device 10 fit to engaging holes 37 of the mouthpiece 30, and the mouthpiece 30 is stably held by the attachment portion 14 (refer to FIGS. 11B and 11C). In addition, in either the first direction or the second direction of the mouthpiece 30, when the mouthpiece 30 is attached to the attachment portion 14, the mouthpiece electrodes 41 of the mouthpiece 30 fit to the owner information reading electrodes 43 of the breath component measuring device 10.

When the mouthpiece 30 is attached, in the first direction, to the attachment portion 14, the mouthpiece direction recognizing contact points 44 are brought in contact with the mouthpiece contact points 42 of the mouthpiece 30. However, when the mouthpiece 30 is attached, in the second direction, to the attachment portion 14, the mouthpiece direction recognizing contact points 44 are not brought in contact with the mouthpiece contact points 42 of the mouthpiece 30. Consequently, the breath component measuring device 10 can recognize the direction of the mouthpiece 30 (the first direction or the second direction) with respect to the breath component measuring device 10.

Next, an internal configuration of the breath component measuring device 10 will be described below. As illustrated in FIG. 14, this breath component measuring device 10 has a gas sensor 15 which measures an alcohol concentration of breath blown in the breath component measuring device 10, a pressure sensor 16 which detects the pressure of breath blown into the breath component measuring device 10, an air barrel 17 which is a container which accumulates breath thereinside and can be expanded and contracted, and a solenoid 18 which contracts the air barrel 17 and guides breath in the air barrel 17 to the gas sensor 15. Further, a flow path of breath in the breath component measuring device 10 includes a flow path 22 which guides the breath blown from a guide hole 21a provided in the projection 21, to a gas sensor room 15a, a flow path 23 which connects the gas sensor room 15a and the air barrel 17 with each other, and a flow path 24 which connects the air barrel 17 and the pressure sensor 16 with each other. The gas sensor room 15a is also part of the flow path, and the gas sensor 15 is arranged inside the gas sensor room 15a.

The air barrel 17 is made from flexible material and is a stretchable and airtight container which has a bellow shape, and the solenoid 18 is a driving mechanism which contracts this air barrel 17. Further, breath is blown into the air barrel 17 in a contracted state, and an air pressure of the breath expands the air barrel 17. Subsequently, the solenoid 18 contracts the air barrel 17, so that the breath accumulated therein is pushed back to the gas sensor room 15a.

The pressure sensor 16 is a detecting unit which detects a pressure of breath blown into the breath component measuring device 10. In the present embodiment, a semiconductor distortion gauge is formed on a surface of a diaphragm, and a semiconductor piezoresistance diffusing pressure sensor which converts a change of electric resistance resulting from a piezoresistance effect produced when the diaphragm deforms due to a force (pressure) from an outside, into an electrical signal is used as the pressure sensor 16.

The gas sensor 15 is a detecting unit which has a gas sensing body, is accommodated inside the gas sensor room 15a of the breath component measuring device 10 and detects gas in breath. At least part of breath blown in the breath flow path is brought in contact with the gas sensing body. In the present embodiment, an electrochemical sensor which has a gas sensing body in which a current flows upon contact with alcohol, and detects the alcohol concentration in the gas according to a value of a flowing current is used as the gas sensor 15. For this electrochemical sensor, for example, Pt or Pt alloys are used as an anode and a cathode, and sulfuric acid (H₂SO₄) is used as electrolyte to measure a change in a current produced when alcohol molecules become oxidized by platinum catalyst.

The gas sensor 15 only needs to detect the alcohol concentration contained in breath, and can be selected from various alcohol sensors such as a semiconductor sensor which detects the alcohol concentration in gas based on electrical resistance which changes according to a reaction of oxygen adsorbed to a metal oxide and alcohol in the gas.

The gas sensor 15 deteriorates in some cases. Hence, the gas sensor 15 is preferably checked up on a regular basis. Although deterioration of the above electrochemical sensor in particular is accelerated depending on conditions, sensors of other types can also deteriorate.

In the present embodiment, the gas sensor 15 starts measuring the alcohol concentration when the pressure sensor 16 detects that breath is continuously blown in the breath component measuring device 10 for a predetermined period of time (for example, five seconds). In addition, start of detection of alcohol by the gas sensor 15 may be judged based on a condition other than a pressure of gas, and, for example, a sensor which detects a carbon dioxide gas concentration in gas in the breath component measuring device 10, an acoustic sensor which detects a sound produced in the mouthpiece 30 which is produced by breath of a subject to be examined or a temperature sensor (thermistor) which detects a temperature of gas in the breath component measuring device 10 is disposed in the breath component measuring device 10 in order to decide whether or not breath is continuously blown in the breath component measuring device 10 for a predetermined period of time.

Further, a control processing unit 100 such as a CPU is provided on a circuit substrate in the breath component measuring device 10. This control processing unit 100 is a computing module which is configured by hardware such as a processor like a CPU or a DSP (Digital Signal Processor), memory and other electronic circuits, software such as a program having a function of the hardware or a combination of these, virtually constructs various functional modules by adequately reading and executing the program, has each constructed functional module control measurement of the above breath component, and controls the entirety of the breath component measuring device 10 such as an input of a power switch 12 or an operation button and an output of a display unit 11a.

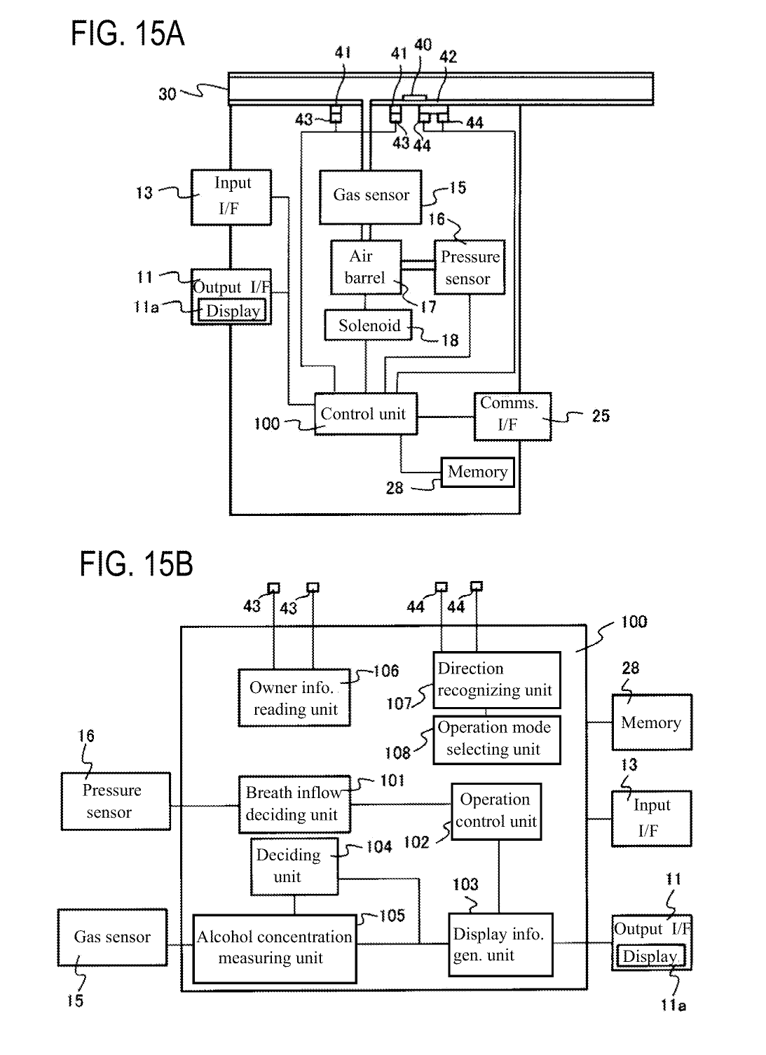

Next, a functional module of the breath component measuring device 10 will be described. FIG. 15A is a block diagram illustrating an entire configuration of the breath component measuring device 10 according to the present embodiment, and FIG. 15B is a block diagram illustrating an internal module of the control processing unit 100. In addition, a “module” which is used in the specification is configured by hardware such as an apparatus or a device, software which has a function thereof or a combination thereof, and means a functional unit for achieving a predetermined operation.

The breath component measuring device 10 has an input interface 13, an output interface 11, a communication interface 25, a memory 28 and the control processing unit 100.

The input interface 13 is a device such as an operation button, a touch panel or a jog dial which receives an input of a user operation. The output interface 11 is a device such as a display or a speaker which outputs an image or a sound. This output interface 11 in particular includes a display unit 11a which is an LCD which displays a measurement result of a detection target gas component contained in breath based on a detection result of the gas sensor 15 and information such as an operation guide.

The communication interface 25 is a communication interface with which the communication cable is connected, and transmits, for example, a measurement result to the information processing terminal connected through the communication cable. Instead of the communication cable, wireless communication such as Bluetooth (registered trademark) may be used.

The memory 28 is a memory device such as ROM or RAM which stores various items of data, and stores a threshold value with respect to the acoustic sensor 16 which determines whether or not the gas sensor 15 starts measurement, a threshold of an alcohol concentration value which determines whether or not driving is allowable and information such as a measurement result.

The control processing unit 100 is a computation processing device such as a CPU, and is a module which virtually configures each functional module by executing various programs on this control processing unit 100. In the present embodiment, the control processing unit 100 has an operation control unit 102, a display information generating unit 103, a deciding unit 104, a breath inflow deciding unit 101, an alcohol concentration measuring unit 105, an owner information reading unit 106, a direction recognizing unit 107 and an operation mode selecting unit 108.

The operation control unit 102 is a module which drives each device of the breath component measuring device 10, and drives, for example, the pressure sensor 16, the gas sensor 15 and the display unit 11a. Particularly, when breath is blown into the breath component measuring device 10 and the pressure sensor 16 continuously detects the pressure exceeding a predetermined threshold, the operation control unit 102 drives the solenoid 18 so as to push breath (gas) in the air barrel 17 back to a gas sensor room 15a. Further, the operation control unit 102 performs control to drive the gas sensor 15 and detect an alcohol concentration.

Furthermore, the operation control unit 102 has a function of controlling an image capturing timing of a still image of a subject using a digital camera 77. More specifically, the operation control unit 102 finishes a first image capturing operation before the subject blows breath into the breath component measuring device 10, and starts a second image capturing operation after the subject blows the sufficient amount of breath in the breath component measuring device 10.

Meanwhile, the first image capturing operation is an operation of capturing a still image of the subject using the digital camera 77 before measurement of the alcohol concentration is started, and, when the power switch 12 of the breath component measuring device 10 is pushed, the operation control unit 102 is activated and transmits a signal which instructs image capturing, to the information processing terminal 7, and the information processing terminal 7 side captures an image of the face of the user within a stand-by time (for example, five seconds) after the power switch 12 is pushed.

Further, the second image capturing operation is an operation of capturing a still image of a subject using the digital camera 77 after acquisition of a breath sample of the subject is finished to measure an alcohol concentration. In the present embodiment, when a certain pressure continues for a predetermined period of time (for example, five seconds) after the pressure sensor 16 detects a certain pressure, the operation control unit 102 transmits a signal which instructs image capture to the information processing terminal 7, and the information processing terminal 7 side captures an image of the subject while the alcohol concentration measuring unit 105 analyzes the alcohol concentration (for example, about three seconds to ten seconds).

The breath inflow deciding unit 101 is a module which calculates a pressure value in the device based on the detection result of the pressure sensor 16, and decides start of an inflow of breath and continuation of breath. More specifically, when the subject blows breath and the pressure sensor 16 detects a predetermined pressure value, the breath inflow deciding unit 101 continuously detects a pressure value in a predetermined period of time while counting the predetermined period of time (for example, about five seconds). Further, when the pressure value detected by the breath inflow deciding unit 101 continuously exceeds the predetermined threshold, the driving control unit 102 drives the solenoid 18, and pushes breath in the air barrel 17 back to the gas sensor room 15a. This is because, when breath which exceeds a certain pressure continues, the amount of air required to measure the alcohol concentration is obtained in the air barrel 17. The subject needs to take a deep breath and exhale breath for a long period of time to obtain an alcohol measurement result.

The alcohol concentration measuring unit 105 is a module for measuring the alcohol concentration which is the detection target gas component contained in breath based on a detection result of the gas sensor 15, and, more specifically, calculates the alcohol concentration in the breath based on a detection result detected when air in the air barrel 17 is sent out to the gas sensor 15 by the operation control unit 102. “Starting measuring the alcohol concentration” referred here is acquiring an output of the gas sensor 15 and starting calculating the alcohol concentration. Meanwhile, the alcohol concentration measuring unit 105 may drive the gas sensor 15 when the pressure level measured by the pressure sensor 16 continuously exceeds the threshold for a certain period of time, and may not drive the gas sensor 15 in other cases. In this case, “starting measuring the alcohol concentration” is starting an output from the gas sensor 15.

The deciding unit 104 is a module which decides whether or not driving is allowable, based on the alcohol concentration of the detection target gas component calculated by the alcohol concentration measuring unit 105. More specifically, the deciding unit 104 compares the calculated alcohol concentration in breath and the threshold stored in the memory 28, and decides that driving is allowable when the calculated alcohol concentration is the threshold or less.

The display information generating unit 103 is a module which displays various pieces of information on the display unit 11a, and, when, for example, the power switch 12 acquires an inputted signal, displays information about an operation guide such as an instruction to start blowing breath or a message to end blowing or displays, for example, a numerical value of the alcohol concentration detected by the gas sensor 15 or a decision result of the deciding unit 104 on the display unit 11a.

When the mouthpiece 30 is attached to an attachment portion 14, the owner information reading unit 106 reads owner information from a memory device 40 of the mouthpiece 30. When the mouthpiece 30 is attached to the attachment portion 14, the owner information reading electrodes 43 contact the mouthpiece electrodes 41 provided in the mouthpiece 30, thereby allowing the owner information reading unit 106 to read owner information from the mouthpiece 30.

The control processing unit 100 associates information about the alcohol concentration obtained from the alcohol concentration measuring unit 105 and subject information, and stores or outputs the information about the alcohol concentration and the subject information. “Store” referred here means storing the information about the alcohol concentration and the subject information in a memory 28 or a memory device which is not illustrated. “Output” referred here means transmitting the information about the alcohol concentration and the subject information to the information processing terminal 7 using the communication interface 25. Further, the control processing unit 100 may output the information about the alcohol concentration and the subject information to the output interface 11 of the breath component measuring device 10.

The subject information referred here may be owner information which is read by the owner information reading unit 106 (owner information stored in the memory device 40) or subject information which corresponds to owner information. The control processing unit 100 may store or output an owner ID read from the memory device 40 or may store or output a name of an owner read from the memory device 40 and a vehicle number used by the owner. Alternatively, the control processing unit 100 may store or output subject information (at least one of, for example, a subject ID, a name of a subject and a vehicle number used by the subject) associated in the memory 28 with the owner ID read from the memory device 40.

The direction recognizing unit 107 recognizes whether the mouthpiece 30 is attached to the attachment portion 14 in a first direction or a second direction. As described above, when the mouthpiece 30 is attached to the attachment portion 14 in the first direction, the mouthpiece direction recognizing contact points 44 contact the mouthpiece contact points 42 of the mouthpiece 30 (the two mouthpiece direction recognizing contact points 44 are electrically connected). However, when the mouthpiece 30 is attached to the attachment portion 14 in the second direction, the mouthpiece direction recognizing contact points 44 do not contact the mouthpiece contact points 42 of the mouthpiece 30 (the two mouthpiece direction recognizing contact points 44 are not electrically connected). Consequently, depending on whether or not a current flows between the two mouthpiece direction recognizing contact points 44, the direction recognizing unit 107 can recognize whether the direction of the mouthpiece 30 with respect to the breath component measuring device 10 is the first direction or the second direction.

The operation mode selecting unit 108 selects an operation mode of the breath component measuring device 10 according to the direction of the mouthpiece 30 recognized by the direction recognizing unit 107. More specifically, when the direction recognizing unit 107 recognizes that the mouthpiece 30 is attached in the first direction, the operation mode selecting unit 108 selects a simple operation mode. When the direction recognizing unit 107 recognizes that the mouthpiece 30 is attached in the second direction, the operation mode selecting unit 108 selects a normal operation mode. However, the operation mode selecting unit 108 may select the normal operation mode with respect to the first posture, and select the simple operation mode with respect to the second posture.

In the normal operation mode, the control processing unit 100 transmits information about an alcohol concentration calculated by the alcohol concentration measuring unit 105, and subject information to the information processing terminal 7. In the simple operation mode, the control processing unit 100 does not transmit the information about the alcohol concentration and the subject information to the information processing terminal 7. In both operation modes, the control processing unit 100 decides whether or not driving is allowable using the deciding unit 104, and the display information generating unit 103 displays the decision result on the display unit 11a.

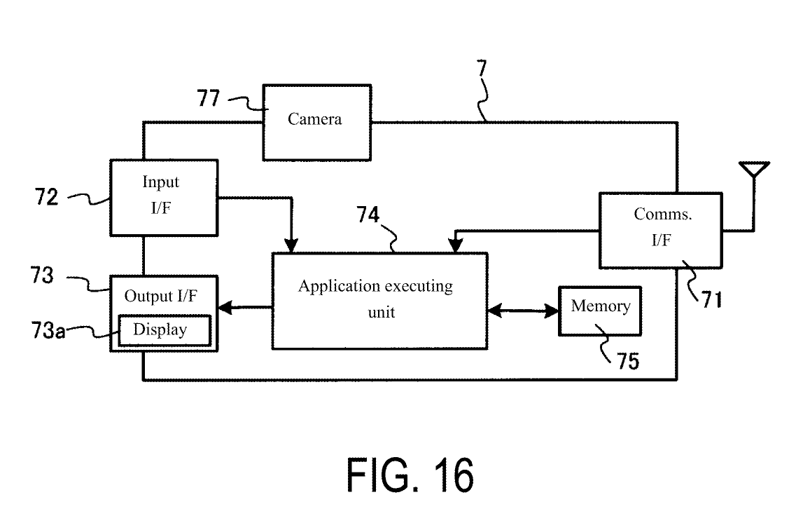

Next, an internal configuration of the information processing terminal 7 will be described. FIG. 16 is a block diagram illustrating an internal configuration of the information processing terminal 7 according to the present embodiment.

As illustrated in FIG. 16, the information processing terminal 7 has an input interface 72 and an output interface 73 as modules of user interfaces. The input interface 72 is a device such as an operation button, a touch panel or a jog dial which receives an input of a user operation. The output interface 73 is a device such as a display or a speaker which outputs an image or a sound. This output interface 73 in particular includes a display unit 73a such as a liquid crystal display.

Further, the information processing terminal 7 has a communication interface 71 as a communication module. The communication interface 71 is a module which transmits and receives various items of data through the communication network such as a mobile telephone communication network or an IP network by way of wireless communication or wired communication.

Further, the information processing terminal 7 has a memory 75 and a digital camera 77. The memory 75 is a memory device which stores various items of data, and, in this memory 75, the measurement result processing program and, in addition, personal information such as a name of the subject to be examined, a telephone number thereof and a vehicle number of use are associated with one another and stored therein. The personal information about the subject is associated with identification information which is used to identify the subject, and the subject may be recognized when, for example, the subject inputs an ID and a password upon measurement or a recording medium in which identification information such as an IC card is recorded is read. The digital camera 77 is an image capturing unit which captures an image of the subject, and captures a still image of the subject under control of the application executing unit 74.

Further, the information processing terminal 7 has the application executing unit 74 as a module which executes an application. The application executing unit 74 is a common OS or a module which executes an application such as browser software or a media viewing application, and is realized by, for example, the CPU.

In the present embodiment, when a measurement result processing program (computer program) is installed, the application executing unit 74 can execute the measurement result processing program. The mobile terminal 7a transmits a measurement result, subject information and position information about the terminal 7a, to a computer installed at a business office of a business operator through a mobile telephone communication network according to the mobile terminal measurement processing program. Meanwhile, the fixed mounted terminal 7b stores in the memory 75 a measurement result and subject identification information according to the fixed mounted terminal measurement result processing program.

A signal transmitted from the breath component measuring device 10 triggers the application executing unit 74 to capture using the digital camera 77 images of the face of the subject before blow of breath is started and after blow is finished. The application executing unit 74 compares image data obtained by capturing images of the faces of the users before and after breath is blown, and decides whether or not the captured user images are match with each other. In this processing, face detection processing of determining a face area from an image and face feature point detection processing of calculating feature point positions of the face such as the eyes, the nose and the corners of the lips are performed to compare the image data before and after breath is blown, to decide whether or not the captured user images match with each other and to output the degree of match of both images which is a comparison result. The degree of match of both of these images may be outputted by producing a warning sound, displaying a message(s) on a display screen and, in addition, sending a message(s) to a predetermined electronic mail address(es) when the users are different from each other. In addition, the application executing unit 74 also has a function of displaying a profile for aligning the position of the face of the subject on the screen of the display unit 73a upon a first image capturing operation and a second image capturing operation, and making an instruction to align the face to this profile.

The mobile terminal 7a sends the image data to the computer installed at the business office of the business operator. The fixed mounted terminal 7b stores image data in the memory 75.

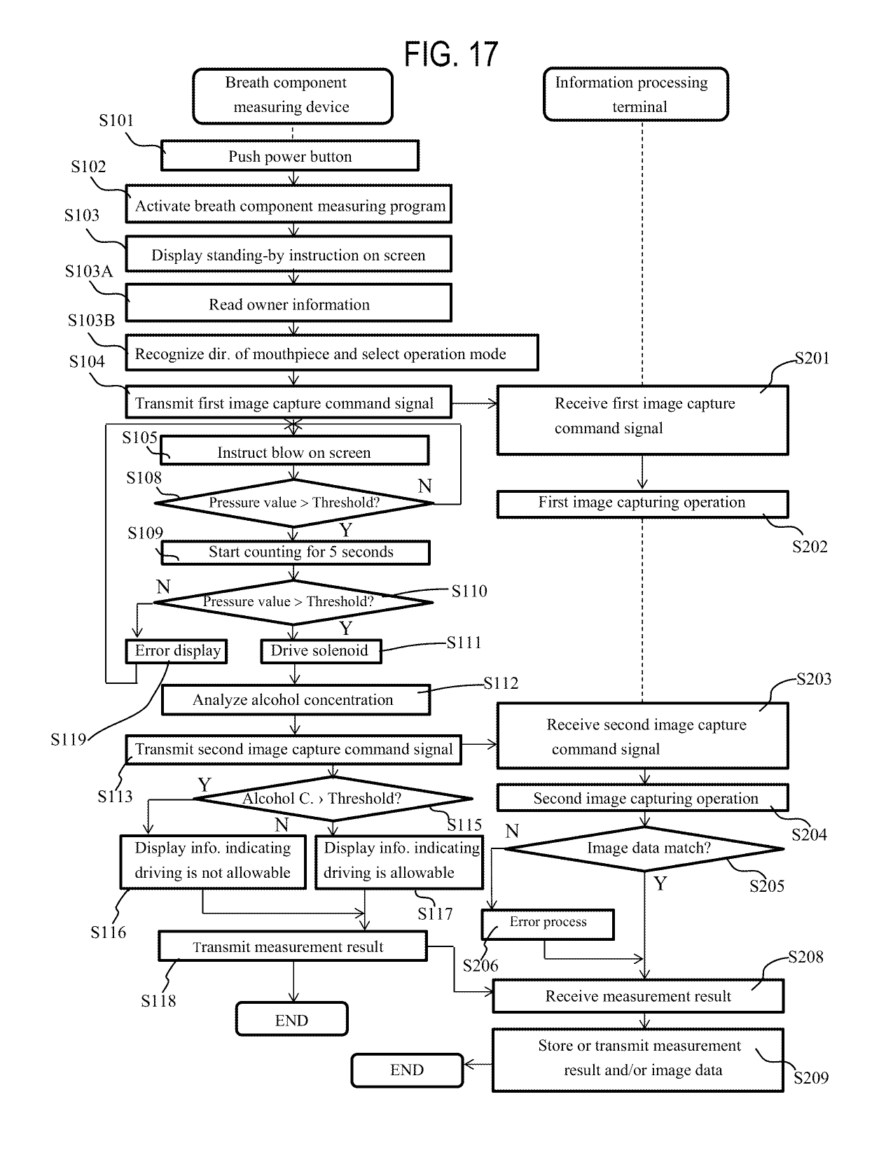

By operating the above-described breath component measuring system, it is possible to implement the breath component measuring method. FIG. 17 is a flowchart illustrating process of the above normal operation mode of the breath component measuring method according to the present embodiment.

As illustrated in FIG. 17, first, when the subject pushes the power switch 12 (S101), the control processing unit 100 activates the breath component measuring program (S102). In this case, the display information generating unit 103 displays a movie of a countdown and an instruction of standing-by for a predetermined period of time (for example, about five seconds) on the display unit 11a until it becomes ready to perform measurement (S103).

The owner information reading unit 106 reads owner information from the memory device 40 of the mouthpiece 30 (S103A). Further, the direction recognizing unit 107 recognizes the direction of the mouthpiece 30, and the operation mode selecting unit 108 selects an operation mode based on the direction of the mouthpiece 30 (S103B). Hereinafter, it is assumed that the normal operation mode is selected.

The operation control unit 102 transmits a first image capture command signal to the information processing terminal 7 (S104). When the information processing terminal 7 receives the first image capture signal (S201), in the standing-by period (for example, five seconds) for start-up of the program, the application executing unit 74 executes a first image capturing operation of capturing an image of the face of the subject using the digital camera 77 (S202). Subsequently, after the stand-by time ends, the display information generating unit 103 of the breath component measuring device 10 displays an instruction to blow breath into the mouthpiece 30 for the predetermined period of time (five seconds) on the screen of the display unit 11a (S105).

Further, when the subject blows breath using the mouthpiece 30, the breath blown from the blow port 31 passes in the breath flow path 33, and the breath flows in the breath component measuring device 10 through a guide hole 21a formed in the projection 21. The breath flows in the breath component measuring device 10, and the breath reaches the pressure sensor 16 through the flow path 24.

The breath inflow deciding unit 101 calculates a pressure value based on the detection result of the pressure sensor 16, and monitors the pressure value. The breath inflow deciding unit 101 first decides whether or not the pressure value detected by the pressure sensor 16 exceeds the predetermined threshold (S108). Meanwhile, when the pressure value does not exceed the threshold (“N” in S108), the breath inflow deciding unit 101 stands by until the pressure value exceeds the threshold.

Meanwhile, when the pressure value exceeds the threshold (“Y” in S108), while counting the predetermined period of time (for example, five seconds) from start of measurement (S109), the breath inflow deciding unit 101 decides whether or not the pressure value detected by the pressure sensor 16 continuously exceeds the predetermined threshold in this predetermined period of time (S110).

When the pressure value does not continuously exceed the threshold, the breath inflow deciding unit 101 decides that the alcohol concentration cannot be measured (“N” in S110). This information is inputted to the display information generating unit 103, and the display information generating unit 103 displays error display and an instruction of redoing of blow on the screen of the display unit 11a (S119).

Meanwhile, when the pressure value detected by the pressure sensor 16 continuously exceeds the predetermined threshold (“Y” in S110), the operation control unit 102 drives the solenoid 18 and sends the air in the air barrel 17 to a gas sensor 15 side (S111).

After a pressure exceeding the threshold is continuously measured, by returning the breath in the air barrel 17 back to the gas sensor 15, it is possible to acquire from the subject the amount of breath which is sufficient to measure alcohol using the gas sensor. The subject needs to take a deep breath and exhale breath for a long period of time to obtain an alcohol measurement result. Even when the subject tries to supply a little amount of breath to the breath component measuring device 10 and obtain a measurement result indicating a low alcohol concentration, redoing of blow is instructed.

The alcohol concentration measuring unit 105 measures (analyzes) the alcohol concentration which is a detection target gas component contained in the breath, based on the detection result of the gas sensor 15 (S112). In this case, the operation control unit 102 controls the output interface 11, and instructs the subject to end blowing breath. Further, the operation control unit 102 transmits a second image capture command signal to the information processing terminal 7 (S113). When the information processing terminal 7 receives the second image capture command signal (S203), the application executing unit 74 executes a second image capturing operation of capturing an image of the subject using the digital camera 77 after measurement of the alcohol concentration ends (S204).

Subsequently, the image comparing unit 74a compares image data (that is, first still image data and second still image data) of the subjects the images of which are captured by the first image capturing operation and the second image capturing operation, and decides whether or not the users of both items of image data match according to face recognition processing (S205). When the users of the image data do not match with each other (“N” in S205), error processing is executed (S206). In the error processing, the image comparing unit 74a generates data which indicates that the users of both images do not match with each other.

In the breath component measuring device 10, when the alcohol concentration measuring unit 105 finishes measuring (analyzing) the alcohol concentration of the detection target gas component, information about the alcohol concentration which is the measurement result is inputted to the deciding unit 104, and the deciding unit 104 compares the calculated alcohol concentration and the predetermined threshold with each other (S115). When the alcohol concentration in breath exceeds the threshold (“Y” in S115), the deciding unit 104 decides that driving is not allowable, and the display information generating unit 103 displays that driving is not allowable, on the screen of the display unit 11a (S116). In this case, the alcohol concentration measurement value may be displayed on the screen of the display unit 11a. Meanwhile, when the alcohol concentration is the threshold or less (“N” in S115), the deciding unit 104 decides that driving is allowable, and the display information generating unit 103 displays that driving is allowable (S117). In this case, the alcohol concentration measurement value may be displayed on the screen of the display unit 11a.

Subsequently, the breath component measuring device 10 transmits information about the measurement result to the information processing terminal 7 through the communication interface 25 (S118). When the information processing terminal 7 receives the measurement result information (S208), the measurement result and/or image data captured twice are stored or sent (S209). More specifically, when the information processing terminal 7 is a fixed mounted terminal 7b, the application executing unit 74 of the fixed mounted terminal 7b stores the measurement result, the image data, the measurement date data and data which indicates an ID or a name of a subject in the memory 75. When the error processing in step S206 is performed (when users the images of which are captured twice do not match with each other), the application executing unit 74 of the fixed mounted terminal 7b stores data which indicates that the users do not match in the memory 75. The fixed mounted terminal 7b may display measurement result information on the screen of the display unit 73a. When the information processing terminal 7 is the mobile terminal 7a, the application executing unit 74 of the mobile terminal 7a transmits a signal indicating the measurement result, image data, measurement date data and data which indicates the ID or the name of the subject, by wireless communication using the communication interface 71 to a computer installed at a business office of a business operator. When the error processing in step S206 is performed (when users images which are captured twice do not match with each other), the application executing unit 74 of the mobile terminal 7a sends a report indicating data which indicates that the users do not match with each other to the computer installed at the business office of the business operator. In the present embodiment, when the users the images of which are obtained by the first image capturing operation and the second image capturing operation do not match with each other, data which indicates that the users in both images do not match with each other is stored in the fixed mounted terminal 7b at the business office of the business operator. Alternatively, the report indicating that the users in both images do no match with each other is sent to the business office of the business operator. Staff at a business office of a business operator learns that a subject changes before and after breath is blown. That is, the staff at the business office of the business operator can learn the existence of an untrustworthy subject who did cheating.

When users in images obtained by the first image capturing operation and the second image capturing operation do not match, the information processing terminal 7 may transmit an error signal to the breath component measuring device 10, and, in the breath component measuring device 10 which receives this error signal, the control processing unit 100 may have the display unit 11a display a warning and the display unit 11a instructs a subject to blow breath again and do alcohol measurement over. That is, the information processing terminal 7 may transmit to the breath component measuring device 10 an error signal which encourages the subject to try alcohol measurement again using the breath component measuring device 10. Further, the breath component measuring device 10 which receives this error signal may produce a warning sound. Even when measurement is done over again, the information processing terminal 7 may store or send data which indicates that users previously did not match.

In S103A in the above process, the owner information reading unit 106 reads owner information from the memory device 40 of the mouthpiece 30. Before the information processing terminal 7 stores or transmits a measurement result and the ID or the name of the subject in S209, the control processing unit 100 of the breath component measuring device 10 transmits subject information (owner information itself as described above or subject information corresponding to the owner information) to the information processing terminal 7 using the communication interface 25. By using the subject information received from the breath component measuring device 10, the information processing terminal 7 can store or send data which indicates the ID or the name of the subject. The control processing unit 100 may transmit the subject information to the information processing terminal 7 at the same time when the first image capture command signal is transmitted (S104), the second image capture command signal is transmitted (S113) or a measurement result is transmitted (S118). Alternatively, the control processing unit 100 may transmit subject information to the information processing terminal 7 at another period.

When the simple operation mode is selected in S103B, the breath component measuring device 10 does not transmit the first image capture command signal (S104), transmit the second image capture command signal (S113) and transmit the measurement result (S118). The breath component measuring device 10 transmits no signal to the information processing terminal 7.

In the above embodiment, an image of a face of a subject is captured using the digital camera 77 of the information processing terminal 7. However, an image capturing operation is not indispensable, and the digital camera 77 is not an indispensable component. Further, transmitting an image capture command signal is not indispensable.

As described above, when the mouthpiece 30 is attached to the attachment portion 14 of the breath component measuring device 10, the owner information reading unit 106 of the breath component measuring device 10 reads owner information from the memory device 40 of the mouthpiece 30. The control processing unit 100 of the breath component measuring device 10 associates information about the alcohol concentration measured by the gas sensor 15 and subject information (owner information or subject information corresponding to the owner information), and stores or outputs the information about the alcohol concentration and the subject information. Consequently, it is possible to store or output the information about the measured alcohol concentration and the subject information without requiring labor to input information for specifying the subject and requiring a special device which inputs this information.

Further, in the above embodiment, when the mouthpiece 30 is attached to the attachment portion 14 of the breath component measuring device 10, the direction recognizing unit 107 recognizes the direction of the mouthpiece 30, and the operation mode selecting unit 108 selects an operation mode of the breath component measuring device 10. Consequently, it is possible to easily switch the operation mode. However, recognizing the direction of the mouthpiece 30 and selecting the operation mode based on the direction are not indispensable, and the mouthpiece direction recognizing contact points 44 of the breath component measuring device 10 and the mouthpiece contact points 42 of the mouthpiece 30 are not indispensable, either.

### Example 2

Next, an EXAMPLE 2 of the SECOND EMBODIMENT according to the present invention will be described. The EXAMPLE 2 is a product of improvement of the EXAMPLE 1, and a breath component measuring assembly 1 according to the EXAMPLE 2 adopts basically the same structure as that of a breath component measuring assembly 1 according to the EXAMPLE 1. An information processing terminal 7 according to the EXAMPLE 2 adopts the same structure as that of the information processing terminal 7 according to the EXAMPLE 1. Meanwhile, a breath component measuring device 10 of the breath component measuring assembly 1 according to the EXAMPLE 2 has a function of recognizing a protective cover as described below.

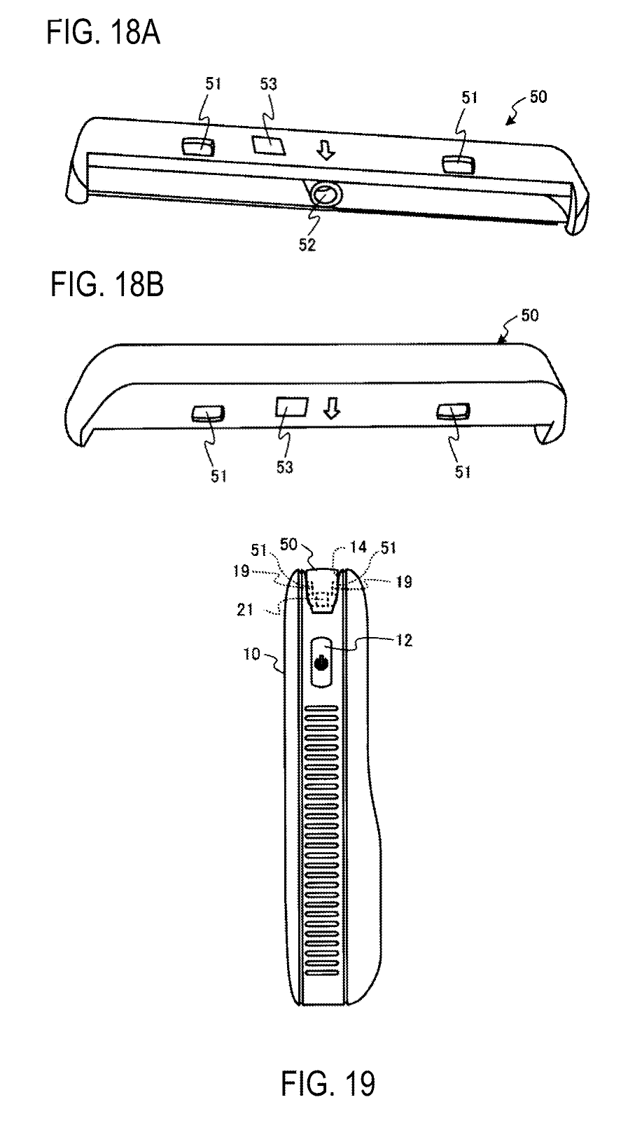

FIG. 18A is a perspective view illustrating a protective cover 50 according to the EXAMPLE 2, which is diagonally viewed from a lower side thereof, and FIG. 18B is a perspective view illustrating the protective cover 50, which is diagonally viewed from an upper side thereof. As illustrated in FIG. 19, the protective cover 50 is detachably attached to the attachment portion 14 of the breath component measuring device 10.

When the breath component measuring device 10 is kept for storage (that is, the breath component measuring device 10 is not used to measure an alcohol concentration), this protective cover 50 is attached to the breath component measuring device 10 to block a guide hole 21a of a projection 21 of the breath component measuring device 10 and protects the gas sensor 15 of the breath component measuring device 10. The protective cover 50 is formed using a hard material such as resin. As illustrated in FIG. 18A, the protective cover 50 has a sealing portion 52. When the protective cover 50 is attached to the breath component measuring device 10, the sealing portion 52 covers and seals the guide hole 21a. The sealing portion 52 is a cylinder which has a hollow portion to which a projection 21 provided with the guide hole 21a fits. As illustrated in FIGS. 18A and 18B, in the protective cover 50, engaging holes 51 are formed which engage with engaging pieces 19 when the protective cover 50 is attached to the breath component measuring device 10.

On an outer surface of the protective cover 50, a protective cover contact point 53 is formed using a conductive material. The protective cover contact point 53 is provided to allow the breath component measuring device 10 to recognize that the protective cover 50 is attached to the breath component measuring device 10.

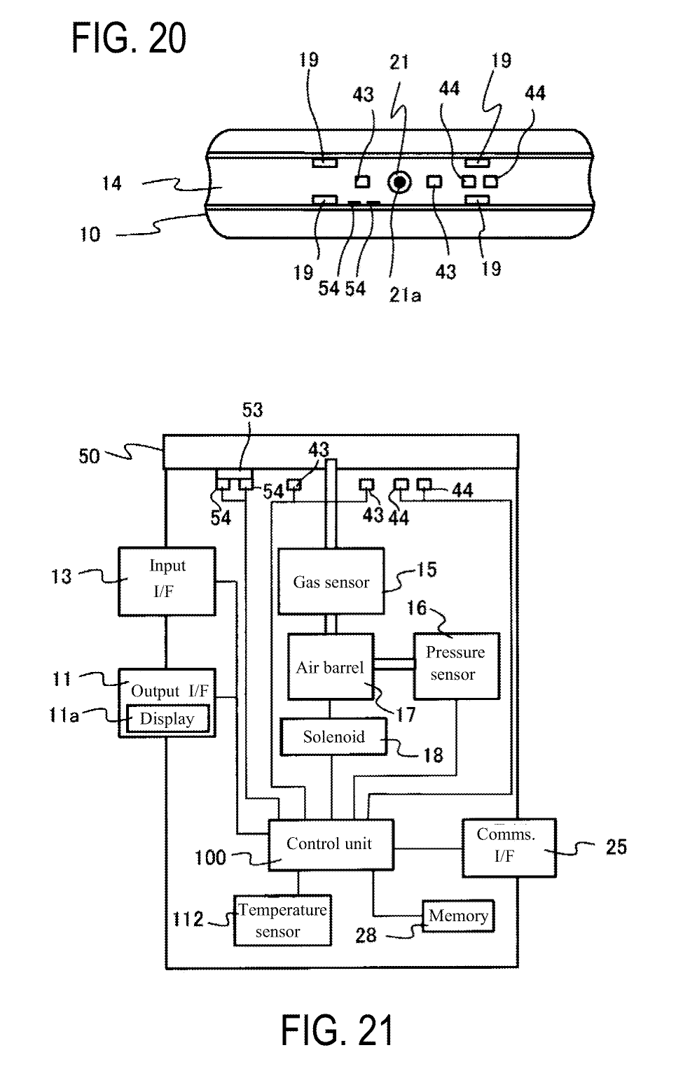

As illustrated in FIG. 20, two protective cover recognizing contact points 54 which can be brought in contact with the protective cover contact point 53 are arranged in the attachment portion 14 of the breath component measuring device 10. The protective cover recognizing contact points 54 are formed using a conductive material. When the protective cover 50 is attached to the attachment portion 14, the protective cover contact point 53 provided in the protective cover 50 is contact with the protective cover recognizing contact points 54, so that the breath component measuring device 10 can recognize the protective cover 50. The protective cover recognizing contact points 54 are arranged at positions different from positions of owner information reading electrodes 43 and mouthpiece direction recognizing contact points 44.

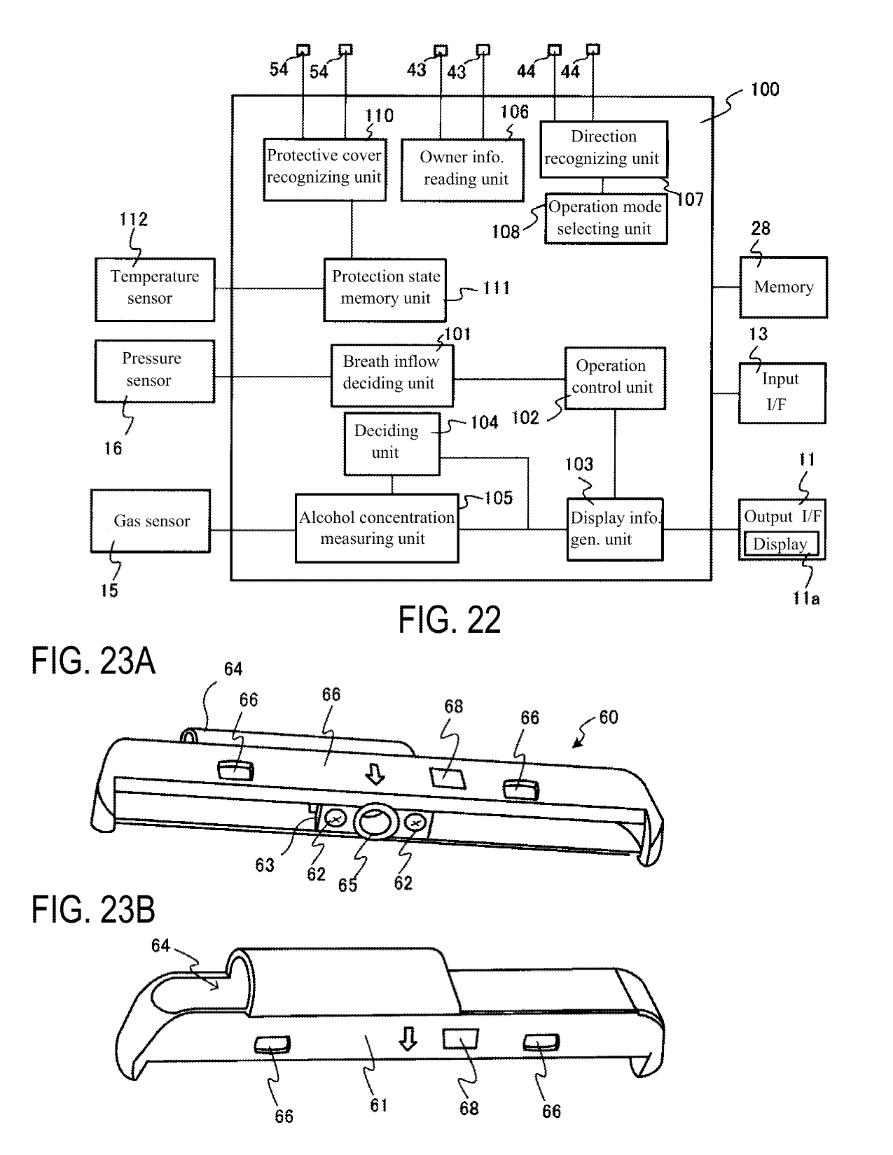

FIG. 21 is a block diagram illustrating an entire configuration of the breath component measuring device 10 according to the present embodiment, and FIG. 22 is a block diagram illustrating an internal module of a control processing unit 100. As illustrated in FIG. 22, the control processing unit 100 has a protective cover recognizing unit 110 and a protection state memory unit 111 in addition to components of the control processing unit 100 according to EXAMPLE 1.

When the protective cover 50 is attached to the attachment portion 14, the protective cover recognizing unit 110 recognizes the protective cover 50. As described above, when the protective cover 50 is attached to the attachment portion 14, the protective cover contact point 53 of the protective cover 50 contacts the two protective cover recognizing contact points 54 of the breath component measuring device 10 (the two protective cover recognizing contact points 54 are electrically connected). When the protective cover 50 is not attached to the attachment portion 14, the protective cover contact point 53 of the protective cover 50 does not contact the two protective cover recognizing contact points 54 of the breath component measuring device 10 (the two protective cover recognizing contact points 54 are not electrically connected). Depending on whether or not current flows between the two protective cover recognizing contact points 54, the protective cover recognizing unit 110 can recognize whether or not the protective cover 50 is attached to the attachment portion 14.

The protection state memory unit 111 stores in the memory 28 a record which indicates whether or not the protective cover 50 is attached to the attachment portion 14 of the breath component measuring device 10, based on the recognition of the protective cover recognizing unit 110. Preferably, in at least a period when the breath component measuring device 10 is not used to measure an alcohol concentration, the protection state memory unit 111 stores a record which indicates whether or not the protective cover 50 is attached to the breath component measuring device 10. That the breath component measuring device 10 is not used to measure the alcohol concentration means that the mouthpiece 30 is not attached to the attachment portion 14. That the mouthpiece 30 is not attached to the attachment portion 14 is decided by an owner information reading unit 106. More specifically, when the owner information reading unit 106 cannot read owner information from the memory device 40 of the mouthpiece 30, the breath component measuring device 10 is not used to measure the alcohol concentration.

More preferably, at a certain cycle, the protection state memory unit 111 stores a record which indicates whether or not the protective cover 50 is attached to the breath component measuring device 10. When the breath component measuring device 10 is not used to measure the alcohol concentration, the control processing unit 100 operates in a sleep mode (a low power consumption state). Also in the sleep mode, the protective cover recognizing unit 110, the protection state memory unit 111 and the owner information reading unit 106 effectively function.

In the present embodiment, in at least a period when the breath component measuring device 10 is not used to measure the alcohol concentration, a record which indicates whether or not the breath component measuring device 10 is adequately kept for storage (whether or not the protective cover 50 blocks the guide hole 21a) is accumulated. The gas sensor 15 and, more particularly, the above electrochemical sensor deteriorate in some cases. By blocking the guide hole 21a of the breath component measuring device 10, it is possible to suppress this deterioration. A record which indicates whether or not the guide hole 21a is blocked is important for a repair man or a manufacturer of the breath component measuring device 10.

The protective cover recognizing contact points 54 are arranged at positions different from positions of owner information reading electrodes 43 and mouthpiece direction recognizing contact points 44. Hence, the control processing unit 100 of the breath component measuring device 10 can easily distinguish which one of the mouthpiece 30 and the protective cover 50 is attached to the attachment portion 14.

In the present embodiment, depending on whether or not a current flows between the two protective cover recognizing contact points 54, the protective cover recognizing unit 110 recognizes whether or not the protective cover 50 is attached to the attachment portion 14. However, by arranging a memory device which stores special information in the protective cover 50, the protective cover recognizing unit 110 may recognize whether or not the protective cover 50 is attached to the attachment portion 14 based on whether or not the protective cover recognizing unit 110 can read this special information.

As illustrated in FIGS. 21 and 22, a temperature sensor 112 which measures the temperature of the breath component measuring device 10 is preferably arranged in a housing of the breath component measuring device 10. The temperature sensor 112 is, for example, a thermistor. An output of the temperature sensor 112 is supplied to the protection state memory unit 111, and the protection state memory unit 111 stores in the memory 28 a record which indicates the temperature. Preferably, in at least a period when the breath component measuring device 10 is not used to measure an alcohol concentration, the protection state memory unit 111 stores a record which indicates the temperature.

The protection state memory unit 111 may store a record which indicates the temperature at a certain cycle. Alternatively, when the temperature exceeds a threshold, the protection state memory unit 111 may record a record which indicates the temperature and a date. Also in a sleep mode, the temperature sensor 112 effectively functions. The record which indicates the temperature indicates whether or not the breath component measuring device 10 is adequately kept for storage, and therefore is important for a repair man or a manufacturer of the breath component measuring device 10. Meanwhile, leaving a record which indicates a temperature is not indispensable, and the temperature sensor 112 used for this purpose is not indispensable, either.

### Example 3

Next, an EXAMPLE 3 of the SECOND EMBODIMENT according to the present invention will be described. The EXAMPLE 3 is a product of improvement of the EXAMPLE 2, and a breath component measuring assembly 1 according to the EXAMPLE 3 adopts basically the same structure as that of a breath component measuring assembly 1 according to the EXAMPLE 2. An information processing terminal 7 according to the EXAMPLE 3 adopts the same structure as that of an information processing terminal 7 according to the EXAMPLE 2. Meanwhile, a breath component measuring device 10 of a breath component measuring assembly 1 according to the EXAMPLE 3 has a function of recognizing a test attachment as described below.

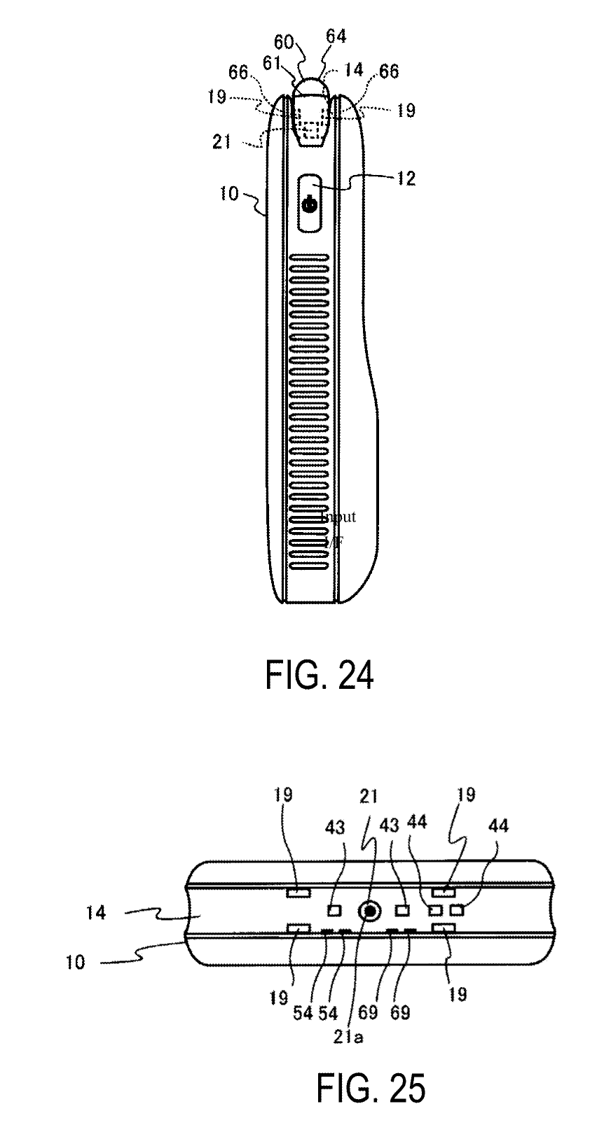

FIG. 23A is a perspective view illustrating a test attachment 60 according to the EXAMPLE 3, which is viewed diagonally from a lower side thereof, and FIG. 23B is a perspective view illustrating a test attachment 60, which is diagonally viewed from an upper side thereof. As illustrated in FIG. 24, the test attachment 60 is detachably attached to the attachment portion 14 of the breath component measuring device 10.

When the breath component measuring device 10 is kept for storage (that is, the breath component measuring device 10 is not used to measure an alcohol measurement), this test attachment 60 is attached to the breath component measuring device 10 to test a gas sensor 15 of the breath component measuring device 10. The gas sensor 15 and, more particularly, the above electrochemical sensor deteriorate in some cases. Upon test of the gas sensor 15, a person uses the test attachment 60 to supply air containing alcohol to the gas sensor 15 in the breath component measuring device 10, and the person or the control processing unit 100 decides whether or not the breath component measuring device 10 normally operates.

The test attachment 60 is formed using a hard material such as resin. As illustrated in FIG. 23A, the test attachment 60 has a long cover 61 and an air guide part 63 which is fixed to the cover 61 by screws 62. In the cover 61, a straw holder 64 is formed. The air guide part 63 has a columnar hollow portion 65. The cover 61 is attached to the attachment portion 14 of the breath component measuring device 10. When the cover 61 is attached to the attachment portion 14, the hollow portion 65 covers a projection 21 of the breath component measuring device 10, and communicates with a guide hole 21a of the projection 21.

An internal space of the straw holder 64 communicates with the hollow portion 65 of the air guide part 63. Hence, when the cover 61 is attached to the attachment portion 14, the internal space of the straw holder 64 communicates with the guide hole 21a of the breath component measuring device 10. The straw holder 64 allows a straw to be inserted. A person who checks up the breath component measuring device 10 attaches the test attachment 60 to the attachment portion 14 of the breath component measuring device 10, sucks gel or a liquid containing alcohol using the straw, inserts the straw in the straw holder 64 and blows the gel or the liquid containing alcohol by means of breath. By this means, air containing alcohol circulates in the air guide part 63, and flows in a flow path in the breath component measuring device 10 from the guide hole 21a.

Meanwhile, a shape and a structure of a test attachment are not limited to the illustrated test attachment 60. For example, the test attachment has a spray, a fan or other parts which guide air containing alcohol to the breath component measuring device 10.

As illustrated in FIGS. 23A and 23B, in the test attachment 60, engaging holes 66 are formed which engage with engaging pieces 19 when the test attachment 60 is attached to the breath component measuring device 10. On the outer surface of the test attachment 60, a test attachment contact point 68 is formed using a conductive material. The test attachment contact point 68 is provided to allow the breath component measuring device 10 to recognize that the test attachment 60 is attached to the breath component measuring device 10.

As illustrated in FIG. 25, two test attachment recognizing contact points 69 which can contact the test attachment contact point 68 are arranged in the attachment portion 14 of the breath component measuring device 10. The test attachment contact points 69 are formed using a conductive material. When the test attachment 60 is attached to the attachment portion 14, the test attachment contact point 68 provided in the test attachment 60 contacts the test attachment recognizing contact points 69, so that the breath component measuring device 10 can recognize the test attachment 60. The test attachment recognizing contact points 69 are arranged at positions different from positions of owner information reading electrodes 43, mouthpiece direction recognizing contact points 44 and the protective cover recognizing contact points 54.

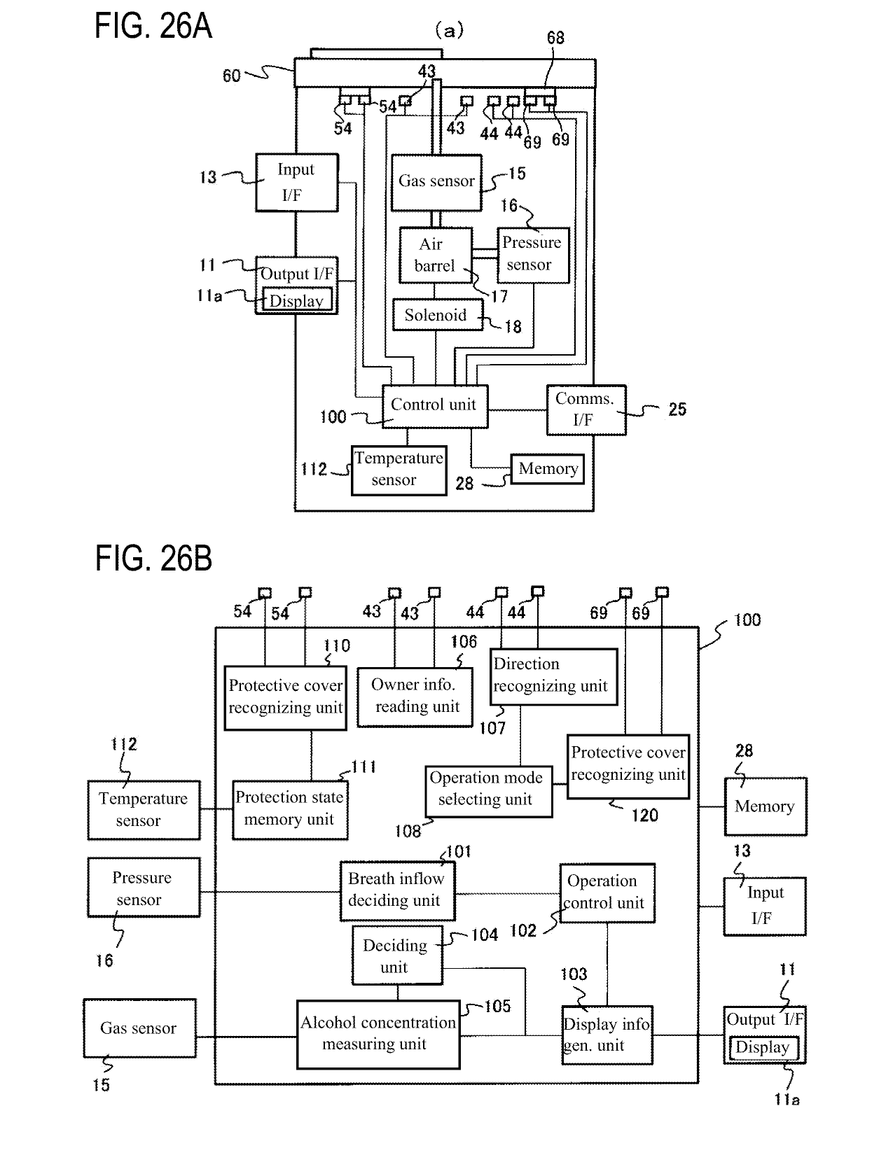

FIG. 26A is a block diagram illustrating an entire configuration of the breath component measuring device 10 according to the present embodiment, and FIG. 26B is a block diagram illustrating an internal module of a control processing unit 100. As illustrated in FIG. 26B, the control processing unit 100 has a test attachment recognizing unit 120 in addition to components of a control processing unit 100 according to the EXAMPLE 2.

When the test attachment 60 is attached to the attachment portion 14, the test attachment recognizing unit 120 recognizes the test attachment 60. As described above, when the test attachment 60 is attached to the attachment portion 14, the test attachment contact point 68 of the test attachment 60 contacts the two test attachment recognizing contact points 69 of the breath component measuring device 10 (the two test attachment recognizing contact points 69 are electrically connected). When the test attachment 60 is not attached to the attachment portion 14, the test attachment contact point 68 of the test attachment 60 does not contact the two test attachment recognizing contact points 69 of the breath component measuring device 10 (the two test attachment recognizing contact points 69 are not electrically connected). Depending on whether or not current flows between the two test attachment recognizing contact points 69, the test attachment recognizing unit 120 can recognize whether or not the test attachment 60 is attached to the attachment portion 14.

An operation mode selecting unit 108 of the control processing unit 100 selects an operation mode based on reading of the owner information reading unit 106 and recognition of the test attachment recognizing unit 120. More accurately, when the owner information reading unit 106 reads owner information, the operation mode selecting unit 108 sets the breath component measuring device 10 to a measurement mode. When the test attachment recognizing unit 120 recognizes the test attachment 60, the operation mode selecting unit 108 sets the breath component measuring device 10 to a test mode.

The measurement mode includes the above normal operation and a simple operation mode in relation to the EXAMPLE 1. In the measurement mode, the control processing unit 100 (sensor output analyzing unit) executes measurement of an alcohol concentration of breath of a subject (including analysis of an output of the gas sensor 15) and various operations related to this measurement.

In the test mode, although the control processing unit 100 (sensor output analyzing unit) analyzes the output of the gas sensor 15, this analysis is performed to check whether or not the gas sensor 15 normally operates. In the test mode, the alcohol concentration calculated by the alcohol concentration measuring unit 105 is outputted by the display information generating unit 103 through the output interface 11. Based on the outputted measurement result, a person may decide whether or not the breath component measuring device 10 is normal. Alternatively, when the alcohol concentration calculated by the alcohol concentration measuring unit 105 is too low, the deciding unit 104 may generate warning information, and the display information generating unit 103 may output the warning information to the output interface 11.

In the present embodiment, depending on whether or not a current flows between the two test attachment recognizing contact points 69, the test attachment recognizing unit 120 can recognize whether or not the test attachment 60 is attached to the attachment portion 14. However, by arranging a memory device which stores special information in the test attachment 60, the test attachment recognizing unit 120 may recognize whether or not the test attachment 60 is attached to the attachment portion 14 depending on whether or not the test attachment recognizing unit 120 can read this special information.

The test attachment recognizing contact points 69 are arranged at positions different from positions of owner information reading electrodes 43, mouthpiece direction recognizing contact points 44 and the protective cover recognizing contact points 54. Hence, the control processing unit 100 of the breath component measuring device 10 can easily distinguish which one of the mouthpiece 30, the protective cover 50 and the test attachment 60 is attached to the attachment portion 14.

The above embodiments may be modified in various modes. For example, the number and an arrangement of electrodes and contact points are not limited to the above embodiment. Components (for example, the protective cover recognizing contact points 54 and the protective cover recognizing unit 110) which recognize the protective cover 50 may be removed from the EXAMPLE 3. Instead of using electrodes, wireless communication may be used to read owner information from the mouthpiece 30.

### Third Embodiment

There are problems that, when an image is captured while the amount of alcohol is measured, a subject decides based on a shutter sound that the measurement ends and stops blowing breath and therefore the amount of alcohol cannot be normally measured and, further, when an image is captured while the amount of alcohol of a subject is measured, the subject puts a mouthpiece in the mouth and performs an operation of blowing air and therefore a facial expression is different from normal facial expression and identification is difficult. Hence, in THIRD EMBODIMENT, provided are a breath component measuring system, a breath component measuring method, a computer program and a breath component measuring device which can measure a gas concentration of an alcohol component contained in breath without a subject interrupting the measurement, makes identification easier by capturing an image of a normal face of the measurement subject and prevent imposter.

The THIRD EMBODIMENT according to the present invention will be described below with reference to the accompanying drawings. In addition, a basic configuration of a breath component measuring system (including an information processing terminal 7) according to the THIRD EMBODIMENT is the same as that of the FIRST EMBODIMENT illustrated in FIGS. 8A and 8B, and a configuration of a mouthpiece 30 and a configuration of connecting the mouthpiece 30 to a breath component measuring device 10 are the same as those of the FIRST EMBODIMENT illustrated in FIGS. 2A, 2B, 3A to 3E and 4A to 4C, and will not be described. In addition, in the THIRD EMBODIMENT, the same configurations as those of the FIRST and SECOND EMBODIMENTS will be assigned the same reference numerals, and configurations different from the FIRST the first and SECOND EMBODIMENTS will be mainly described below.

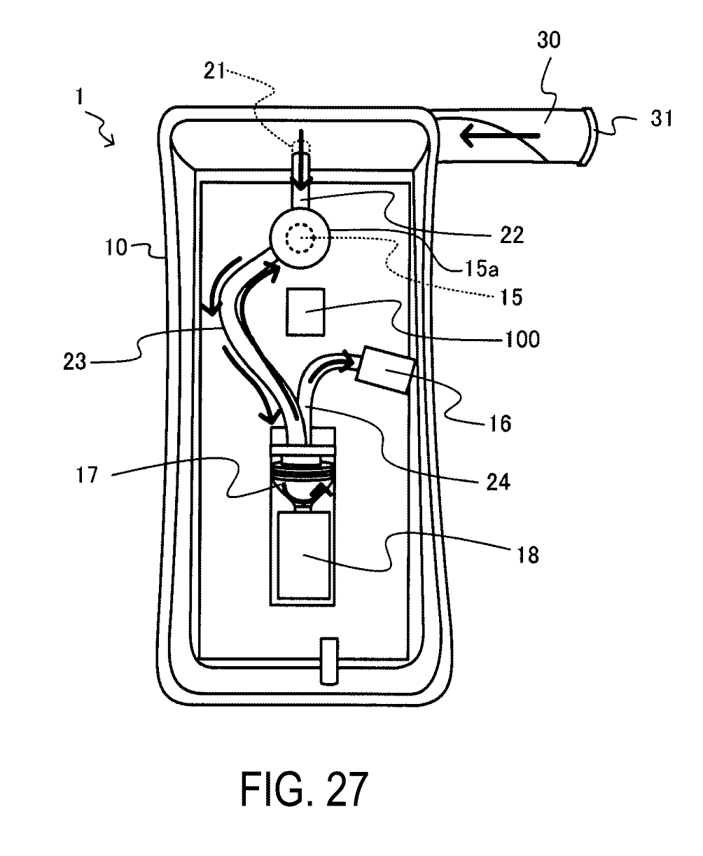

First, an internal configuration of the breath component measuring device 10 will be described. As illustrated in FIG. 27, the breath component measuring device 10 has a gas sensor 15 which measures an alcohol concentration of breath blown in a breath flow path 33, a pressure sensor 16 which detects the pressure of breath blown in the breath component measuring device 10, an air barrel 17 which is a container which accumulates breath inside and can swell and contract, and a solenoid 18 which contracts the air barrel 17 and guides breath inside the air barrel 17 to the gas sensor 15. Further, a flow path of breath inside the breath component measuring device 10 includes a flow path 22 which guides breath blown from a guide hole provided in a projection 21, to a gas sensor room 15a, a flow path 23 which connects the gas sensor room 15a and the air barrel 17 and a flow path 24 which connects the air barrel 17 and the pressure sensor 16. The gas sensor room 15a is also part of a flow path, and the gas sensor 15 is arranged inside the gas sensor room 15a.

The pressure sensor 16 is a detecting unit which detects a pressure of breath blown in the breath component measuring device 10. In the present embodiment, a semiconductor distortion gauge is formed on a surface of a diaphragm, and a semiconductor piezoresistance diffusing pressure sensor which converts a change of electric resistance resulting from a piezoresistance effect produced when the diaphragm deforms due to a force (pressure) from an outside, into an electrical signal is used as the pressure sensor 16.

The gas sensor 15 is a detecting unit which has a gas sensing body, is accommodated inside the gas sensor room 15a of the breath component measuring device 10 and detects gas in breath. At least part of breath blown in the breath flow path contacts the gas sensing body. In the present embodiment, an electrochemical sensor which has a gas sensing body in which a current flows upon contact with alcohol, and detects the alcohol concentration in the gas according to a value of a flowing current is used as the gas sensor 15. For this electrochemical sensor, for example, Pt or Pt alloys are used as an anode and a cathode, and sulfuric acid (H₂SO₄) is used as electrolyte to measure a change in a current produced when alcohol molecules become oxidized by platinum catalyst.

In addition, the gas sensor 15 only needs to detect the alcohol concentration contained in breath, and can adopt alcohol sensors of various systems such as a semiconductor sensor which detects the alcohol concentration in gas based on electrical resistance which changes according to a reaction of oxygen adsorbed to a metal oxide and alcohol in the gas.

In the present embodiment, the gas sensor 15 starts measuring the alcohol concentration when the pressure sensor 16 detects that breath is continuously blown in the breath component measuring device 10 for a predetermined period of time (for example, five seconds). In addition, starting detecting alcohol by means of the gas sensor 15 may be decided based on a condition other than a pressure of gas, and, for example, a sensor which detects a carbon dioxide gas concentration in gas in the breath component measuring device 10, an acoustic sensor which detects a sound produced in the mouthpiece 30 which is produced by breath of a subject or a temperature (thermistor) which detects a temperature of gas in the breath component measuring device 10 is disposed in the breath component measuring device 10 to decide whether or not breath is continuously blown in the breath component measuring device 10 for a predetermined period of time.

Further, a control unit 100 such as a CPU is provided on a circuit substrate in the breath component measuring device 10. This control unit 100 is a computing module which is configured by hardware such as a processor like a CPU or a DSP (Digital Signal Processor), memory and other electronic circuits, software such as a program having a function of the hardware or a combination of these, virtually constructs various functional modules by adequately reading and executing the program, has each constructed functional module control measurement of the above breath component, and controls the entirety of the breath component measuring device 10 such as an input of a power switch 12 or an operation button and an output of a display unit 11a.

### Breath Measuring Functional Module of Breath Component Measuring Device 10

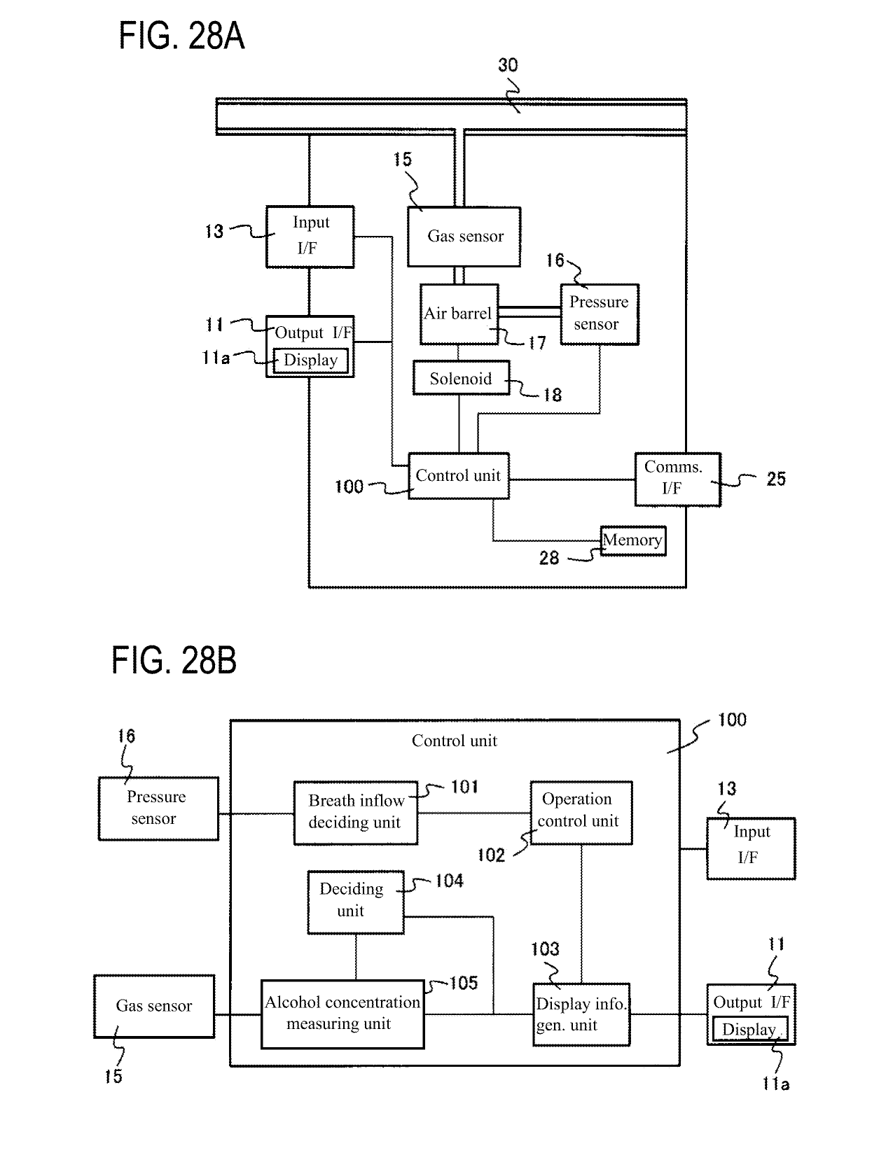

Next, a functional module of the breath component measuring device 10 will be described. FIG. 28A is a block diagram illustrating an entire configuration of the breath component measuring device 10 according to the present embodiment, and FIG. 28B is a block diagram illustrating an internal module of the control unit 100. In addition, a “module” which is used in this description is configured by hardware such as an apparatus or a device, software which has a function of the apparatus or the device or a combination of these, and refers to a functional unit which achieves a predetermined operation.

The breath component measuring device 10 has an input interface 13, an output interface 11, a communication interface 25, a memory 28 and the control unit 100.

The input interface 13 is a device such as an operation button, a touch panel or a jog dial which receives an input of a user operation. The output interface 11 is a device such as a display or a speaker which outputs an image or a sound. This output interface 11 in particular includes a display unit 11a which is an LCD which displays a measurement result of a detection target gas component contained in breath based on a detection result of the gas sensor 15 and information such as an operation guide.

The communication interface 25 is a communication interface with which the communication cable is connected, and transmits, for example, a measurement result to the information processing terminal connected through the communication cable. Instead of the communication cable, wireless communication such as Bluetooth (registered trademark) may be used.

The memory 28 is a memory device such as ROM or RAM which stores various items of data, and stores a threshold value with respect to the acoustic sensor 16 which determines whether or not the gas sensor 15 starts measurement, a threshold of an alcohol concentration value which determines whether or not driving is allowable and information such as a measurement result.

The control unit 100 is a computation processing device such as a CPU, and is a module which virtually constructs each functional module by executing various programs on this control unit 100. In the present embodiment, the control unit 100 has an operation control unit 102, a display information generating unit 103, a deciding unit 104, a breath inflow deciding unit 101 and an alcohol concentration measuring unit 105.

The operation control unit 102 is a module which drives each device of the breath component measuring device 10, and drives, for example, the pressure sensor 16, the gas sensor 15 and the display unit 11a. Particularly, when breath is blown in the breath component measuring device 10 and the pressure sensor 16 continuously detects the pressure exceeding a predetermined threshold, the operation control unit 102 drives the solenoid 18 and pushes breath in the air barrel 17 back to a gas sensor room 15a. Further, the operation control unit 102 performs control to drive the gas sensor 15 and detect an alcohol concentration.

Furthermore, the operation control unit 102 has a function of controlling an image capturing timing of a still image of a subject using a digital camera 77. More specifically, the operation control unit 102 finishes a first image capturing operation before the subject blows breath in the breath component measuring device 10, and starts a second image capturing operation after the subject blows the sufficient amount of breath in the breath component measuring device 10.

Meanwhile, the first image capturing operation is an operation of capturing a still image of the subject using the digital camera 77 before measurement of the alcohol concentration is started, and, when the power switch 12 of the breath component measuring device 10 is pushed, the operation control unit 102 is activated and transmits a signal which instructs image capturing, to the information processing terminal 7, and the information processing terminal 7 side captures an image of the face of the user within a stand-by time (for example, five seconds) after the power switch 12 is pushed.

Further, the second image capturing operation is an operation of capturing a still image of a subject using the digital camera 77 after acquisition of a breath sample of the subject is finished to measure an alcohol concentration. In the present embodiment, when a certain pressure continues for a predetermined period of time (for example, five seconds) after the pressure sensor 16 detects a certain pressure, the operation control unit 102 transmits a signal which instructs image capturing to the information processing terminal 7, and the information processing terminal 7 side captures an image of the subject while the alcohol concentration measuring unit 105 analyzes the alcohol concentration (for example, about three seconds to ten seconds).

The breath inflow deciding unit 101 is a module which calculates a pressure value in the device based on the detection result of the pressure sensor 16, and decides start of an inflow of breath and continuation of breath. More specifically, when the subject blows breath and the pressure sensor 16 detects a predetermined pressure value, the breath inflow deciding unit 101 continuously detects a pressure value in a predetermined period of time while counting the predetermined period of time (for example, about five seconds). Further, when the pressure value detected by the breath inflow deciding unit 101 continuously exceeds the predetermined threshold, the driving control unit 102 drives the solenoid 18, and pushes breath in the air barrel 17 back to the gas sensor room 15a. This is because, when breath which exceeds a certain pressure continues, the amount of air required to measure the alcohol concentration is obtained in the air barrel 17. The subject needs to take a deep breath and exhale breath for a long period of time to obtain an alcohol measurement result.

The alcohol concentration measuring unit 105 is a module which measures the alcohol concentration which is the detection target gas component contained in breath based on a detection result of the gas sensor 15, and, more specifically, calculates the alcohol concentration in breath based on a detection result detected when air in the air barrel 17 is sent out to the gas sensor 15 by the operation control unit 102. “Starting measuring the alcohol concentration” referred here is acquiring an output of the gas sensor 15 and starting calculating the alcohol concentration. Meanwhile, the alcohol concentration measuring unit 105 may drive the gas sensor 15 when the pressure level measured by the pressure sensor 16 continuously exceeds the threshold for a certain period of time, and may not drive the gas sensor 15 in other cases. In this case, “starting measuring the alcohol concentration” is starting an output from the gas sensor 15.

The deciding unit 104 is a module which decides whether or not driving is allowable, based on the alcohol concentration of the detection target gas component calculated by the alcohol concentration measuring unit 105. More specifically, the deciding unit 104 compares the calculated alcohol concentration in breath and the threshold stored in the memory 28, and decides that driving is allowable when the calculated alcohol concentration is the threshold or less.

The display information generating unit 103 is a module which displays various pieces of information on the display unit 11a, and, when, for example, the power switch 12 acquires an inputted signal, displays information about an operation guide such as an instruction to start blowing breath or a message to end blowing or displays, for example, a numerical value of the alcohol concentration detected by the gas sensor 15 or a decision result of the deciding unit 104 on the display unit 11a.

### Internal Configuration of Information Processing Terminal 7

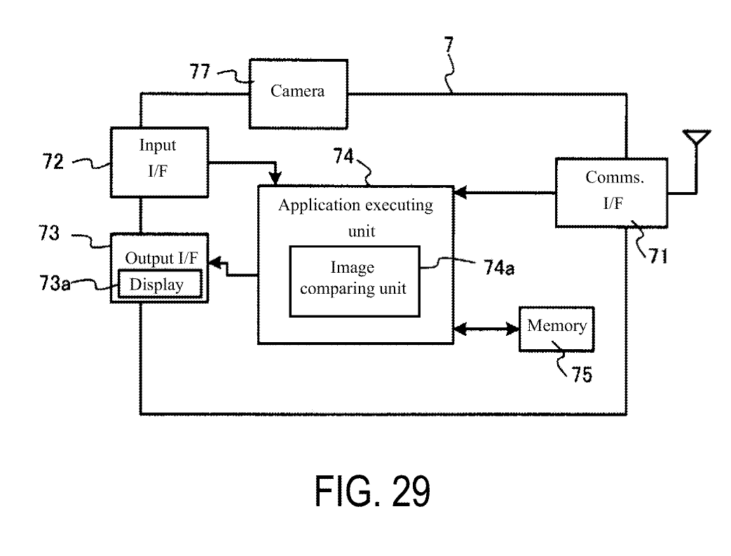

Next, an internal configuration of the information processing terminal 7 will be described. FIG. 29 is a block diagram illustrating an internal configuration of the information processing terminal 7 according to the present embodiment.

As illustrated in FIG. 29, the information processing terminal 7 has an input interface 72 and an output interface 73 as modules of user interfaces. The input interface 72 is a device such as an operation button, a touch panel or a jog dial which receives an input of a user operation. The output interface 73 is a device such as a display or a speaker which outputs an image or a sound. This output interface 73 in particular includes a display unit 73a such as a liquid crystal display.

Further, the information processing terminal 7 has a communication interface 71 as a communication module. The communication interface 71 is a module which transmits and receives various items of data through a communication network such as a mobile telephone communication network or an IP network by way of wireless communication or wired communication.

Further, the information processing terminal 7 has a memory 75 and a digital camera 77. The memory 75 is a memory device which stores various items of data, and, in this memory 75, the measurement result processing program and, in addition, personal information such as a name of the subject, a telephone number and a vehicle number of use are associated and stored. The personal information about the subject is associated with identification information which is used to identify the subject, and the subject may be recognized when, for example, the subject inputs an ID and a password upon measurement or a recording medium in which identification information such as an IC card is recorded is read. The digital camera 77 is an image capturing unit which captures an image of the subject, and captures a still image of the subject under control of the application executing unit 74.

Further, the information processing terminal 7 has the application executing unit 74 as a module which executes an application. The application executing unit 74 is a common OS or a module which executes an application such as browser software or a media viewing application, and is realized by, for example, the CPU.

In the present embodiment, when a measurement result processing program (computer program) is installed, the application executing unit 74 can execute the measurement result processing program. The mobile terminal 7a transmits a measurement result, identification information which is used to specify the subject and position information about the terminal 7a, to a computer installed at a business office of a business operator through a mobile telephone communication network according to the mobile terminal measurement processing program. Meanwhile, the fixed mounted terminal 7b stores in the memory 75 a measurement result and identification information which is used to specify the subject according to the fixed mounted terminal measurement result processing program. The application executing unit 74 has an image comparing unit 74a as a module which compares a first still image captured by a first image capturing operation and a second still image captured by a second image capturing operation.

A signal transmitted from the breath component measuring device 10 triggers the application executing unit 74 to capture using the digital camera 77 an image of the face of the subject before blow of breath is started and after blow is finished. The image comparing unit 74a compares image data obtained by capturing the faces of the users before and after breath is blown, and decides whether or not the users the images of which are captured match. In this processing, face detection processing of determining a face area from an image and face feature point detection processing of calculating feature point positions of the face such as the eyes, the nose and the corners of the lips are performed to compare image data before and after breath is blown, decide whether or not the users the images of which are captured match and output the degree of match of both images which is a comparison result. The degree of match of both of these images may be outputted by producing a warning sound, displaying a message on a display screen and, in addition, sending a message to a predetermined electronic mail address when the users are different. In addition, the application executing unit 74 also has a function of displaying a profile for aligning the position of the face of the subject on the screen of the display unit 73a upon a first image capturing operation and a second image capturing operation, and making an instruction to align the face to this profile. The mobile terminal 7a sends image data to the computer installed at the business office of the business operator. The fixed mounted terminal 7b stores image data in the memory 75.

### Breath Component Measuring Method

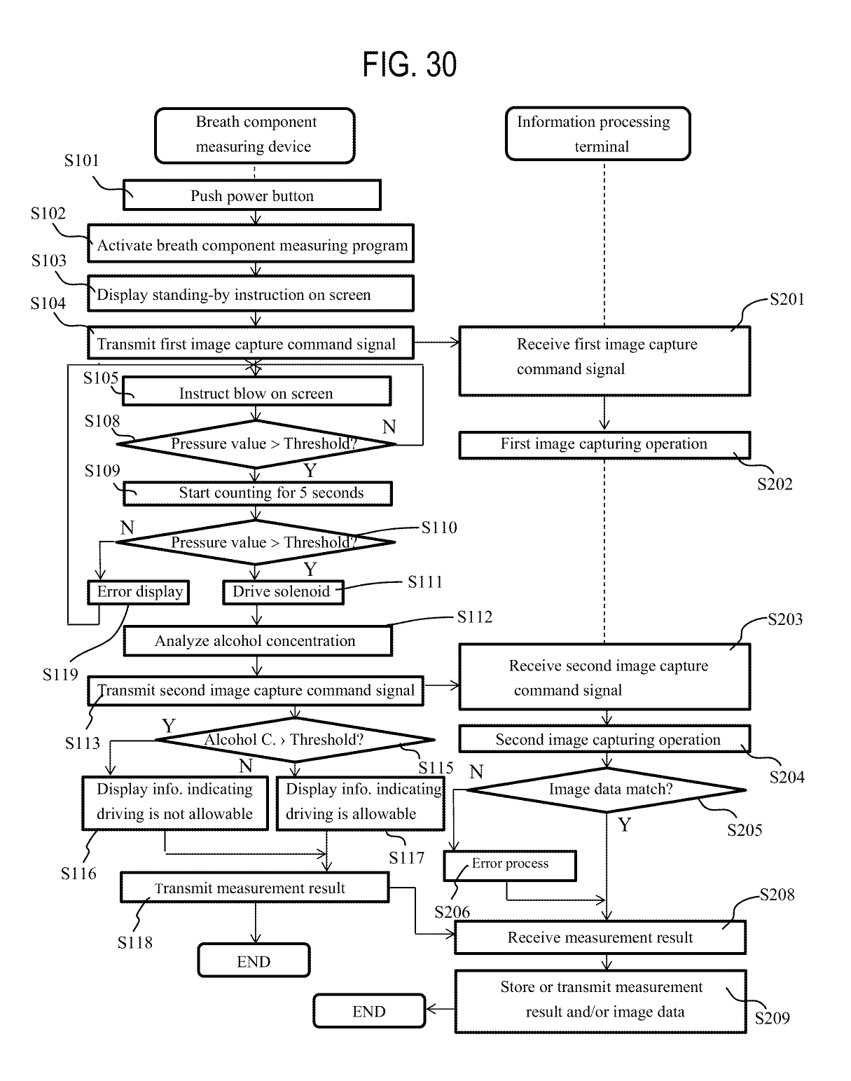

By operating the above breath component measuring system, it is possible to implement the breath component measuring method according to an embodiment of the present invention. FIG. 30 is a flowchart illustrating the breath component measuring method according to the present embodiment.

As illustrated in FIG. 30, first, when the subject pushes the power switch 12 (S101), the control unit 100 activates the breath component measuring program (S102). In this case, the display information generating unit 103 displays a movie of a countdown and an instruction of stand-by for a predetermined period of time (for example, about five seconds) on the display unit 11a until measurement can be performed (S103).

The operation control unit 102 transmits a first image capture command signal to the information processing terminal 7 (S104). When the information processing terminal 7 receives the first image capture signal (S201), in the stand-by time (for example, five seconds) upon launch of the program, the application executing unit 74 executes a first image capturing operation of capturing an image of the face of the subject using the digital camera 77 (S202). Subsequently, after the stand-by time ends, the display information generating unit 103 of the breath component measuring device 10 displays an instruction to blow breath in the mouthpiece 30 for the predetermined period of time (five seconds) on the screen of the display unit 11a (S105).

Further, when the subject blows breath using the mouthpiece 30, the breath blown from the blow port 31 passes in the breath flow path 33, and the breath flows in the breath component measuring device 10 through a guide hole formed in the projection 21. The breath flows in the breath component measuring device 10, and the breath reaches the pressure sensor 16 through the flow path 24.

The breath inflow deciding unit 101 calculates a pressure value based on the detection result of the pressure sensor 16, and monitors the pressure value. The breath inflow deciding unit 101 first decides whether or not the pressure value detected by the pressure sensor 16 exceeds the predetermined threshold (S108). Meanwhile, when the pressure value does not exceed the threshold (“N” in S108), the breath inflow deciding unit 101 stands by until the pressure value exceeds the threshold.

Meanwhile, when the pressure value exceeds the threshold (“Y” in S108), while counting the predetermined period of time (for example, five seconds) from start of measurement (S109), the breath inflow deciding unit 101 decides whether or not the pressure value detected by the pressure sensor 16 continuously exceeds the predetermined threshold in this predetermined period of time (S110).

When the pressure value does not continuously exceed the threshold, the breath inflow deciding unit 101 decides that the alcohol concentration cannot be measured (“N” in S110). This information is inputted to the display information generating unit 103, and the display information generating unit 103 displays error display and an instruction of redoing of blow on the screen of the display unit 11a (S119).

Meanwhile, when the pressure value detected by the pressure sensor 16 continuously exceeds the predetermined threshold (“Y” in S110), the operation control unit 102 drives the solenoid 18 and sends the air in the air barrel 17 to a gas sensor 15 side (S111).

By returning the breath in the air barrel 17 back to the gas sensor 15 after a pressure exceeding the threshold is continuously measured, it is possible to acquire from the subject the amount of breath which is sufficient to measure alcohol using the gas sensor. The subject needs to take a deep breath and exhale breath for a long period of time to obtain an alcohol measurement result. Even when the subject tries to supply a little amount of breath to the breath component measuring device 10 and obtain a measurement result indicating a low alcohol concentration, redoing of blow is instructed.

The alcohol concentration measuring unit 105 measures (analyzes) the alcohol concentration which is a detection target gas component contained in the breath, based on the detection result of the gas sensor 15 (S113). In this case, the operation control unit 102 controls the output interface 11, and instructs the subject to end blowing breath. Further, the operation control unit 102 transmits a second image capture command signal to the information processing terminal 7 (S113). When the information processing terminal 7 receives the second image capture command signal (S203), the application executing unit 74 executes a second image capturing operation of capturing an image of the subject using the digital camera 77 after measurement of the alcohol concentration ends (S204).

Subsequently, the image comparing unit 74a compares image data (that is, first still image data and second still image data) of the subjects the images of which are captured by the first image capturing operation and the second image capturing operation, and decides whether or not the users of both items of image data match according to face recognition processing (S205). When the users of the image data do not match (“N” in S205), error processing is executed (S206). In the error processing, the image comparing unit 74a generates data which indicates that the users of both images do not match.

In the breath component measuring device 10, when the alcohol concentration measuring unit 105 finishes measuring (analyzing) the alcohol concentration of the detection target gas component, information about the alcohol concentration which is the measurement result is inputted to the deciding unit 104, and the deciding unit 104 compares the calculated alcohol concentration and the predetermined threshold (S115). When the alcohol concentration in breath exceeds the threshold (“Y” in S115), the deciding unit 104 decides that driving is not allowable, and the display information generating unit 103 displays that driving is not allowable, on the screen of the display unit 11a (S116). In this case, the alcohol concentration measurement value may be displayed on the screen of the display unit 11a. Meanwhile, when the alcohol concentration is the threshold or less (“N” in S115), the deciding unit 104 decides that driving is allowable, and the display information generating unit 103 displays that driving is allowable (S117). In this case, the alcohol concentration measurement value may be displayed on the screen of the display unit 11a.

Subsequently, the breath component measuring device 10 transmits information about the measurement result to the information processing terminal 7 through the communication interface 25 (S118). When the information processing terminal 7 receives the measurement result information (S208), the measurement result and image data captured twice are stored or sent (S209). More specifically, when the information processing terminal 7 is a fixed mounted terminal 7b, the application executing unit 74 of the fixed mounted terminal 7b stores a measurement result, image data, measurement date data and data which indicates a name of a subject in the memory 75. When the error processing in step S206 is performed (when users the images of which are captured twice do not match), the application executing unit 74 of the fixed mounted terminal 7b stores data which indicates that the users do not match either in the memory 75. The fixed mounted terminal 7b may display measurement result information on the screen of the display unit 73a. When the information processing terminal 7 is the mobile terminal 7a, the application executing unit 74 of the mobile terminal 7a transmits a signal indicating the measurement result, image data, measurement date data and data which indicates the name of the subject, by radio using the communication interface 71 to a computer installed at a business office of a business operator. When the error processing in step S206 is performed (when users the images of which are captured twice do not match), the application executing unit 74 of the mobile terminal 7a sends a report indicating data which indicates that the users do not match either to the computer installed at the business office of the business operator. In the present embodiment, when the users the images of which are obtained by the first image capturing operation and the second image capturing operation do not match, data which indicates that the users in both images do not match is stored in the fixed mounted terminal 7b at the business office of the business operator. Alternatively, the report indicating that the users in both images do no match is sent to the business office of the business operator. Staff at a business office of a business operator learns that a subject changes before and after breath is blown. That is, staff at a business office of a business operator can learn the existence of an untrustworthy subject who did cheating.

In addition, although the breath component measuring device 10 decides whether or not driving is allowable and the breath component measuring device 10 displays, for example, a result in the present embodiment, the present invention is not limited to this, and may transmit an alcohol concentration measurement result to the information processing terminal 7, have the information processing terminal 7 side decide whether or not driving is allowable and have the display unit 73a of the information processing terminal 7 side display the decision result as to whether or not driving is allowable.

When users in images obtained by the first image capturing operation and the second image capturing operation do not match, the information processing terminal 7 may transmit an error signal to the breath component measuring device 10, and, in the breath component measuring device 10 which receives this error signal, the control unit 100 may have the display unit 11a display a warning and the display unit 11a may instruct a subject to blow breath again and do alcohol measurement over. That is, the information processing terminal 7 may transmit to the breath component measuring device 10 an error signal which encourages the subject to try alcohol measurement again using the breath component measuring device 10. Further, the breath component measuring device 10 which receives this error signal may produce a warning sound. Even when measurement is done over again, the information processing terminal 7 may store or send data which indicates that users previously did not match.

Further, although the digital camera 77 of the information processing terminal 7 captures an image of the face of the subject in the above embodiment, for example, the breath component measuring device 10 may have a camera and the breath component measuring device 10 may capture an image of the face of a subject.

### Measurement Result Processing Program

The breath component measuring method according to the present embodiment can be realized by executing a measurement result processing program described according to a predetermined language on a general-use computer such as a mobile computer, a smartphone or a table PC which can communicate with the breath component measuring device 10. More specifically, the following steps are executed in the computer, that is, the information processing terminal 7.

The information processing terminal 7 executes a first image capturing operation step of capturing a first still image of a user using the digital camera 77 according to a first image capture command signal supplied from the breath component measuring device 10 before breath is blown in the breath component measuring device 10. The information processing terminal 7 executes a second image capturing operation step of capturing a second still image of a user using the digital camera 77 according to a second image capture command signal supplied from the breath component measuring device 10 after breath is blown in the breath component measuring device 10. The information processing terminal 7 executes an image comparing step of comparing the first still image and the second still image, and deciding the degree of match of the user of the first still image and the user of the second still image.

Further, by installing the computer in the information processing terminal 7 and executing the computer program on the CPU, it is possible to easily construct a system which has each of the above functions. This program can be distributed through, for example, a communication line, or can be delivered as a package application.

### Fourth Embodiment

Although in a tester which inspects an alcohol checker, alcohol gas is used, alcohol gas is expensive. Further, a disinfectant alcohol liquid or a liquid oral cleaner has a high alcohol concentration, and therefore there is a concern that a sensor which measures alcohol inside a breath component measuring device deteriorates. Hence, a low-cost method is desired in order to supply air containing alcohol (ethanol) of an adequate concentration to the breath component measuring device. Hence, in a FOURTH EMBODIMENT, provided are a test device of a breath component measuring device, a test attachment and a breath component measuring assembly which supply air containing alcohol of an adequate concentration to the breath component measuring device at low cost.

The FOURTH EMBODIMENT of the present invention will be described below with reference to the accompanying drawings. In addition, a basic configuration of a breath component measuring system (including an information processing terminal 7) according to the FOURTH EMBODIMENT is the same as that of a breath component measuring system according to the FIRST EMBODIMENT illustrated in FIGS. 8A and 8B, and will not be described. Further, in the FOURTH EMBODIMENT, the same configurations as in those of the FIRST, SECOND and THIRD EMBODIMENTS will be assigned the same reference numerals, and configurations different from those of the FIRST, SECOND and THIRD EMBODIMENTS will be mainly described.

### Example 1

An EXAMPLE 1 of the FOURTH EMBODIMENT will be described below.

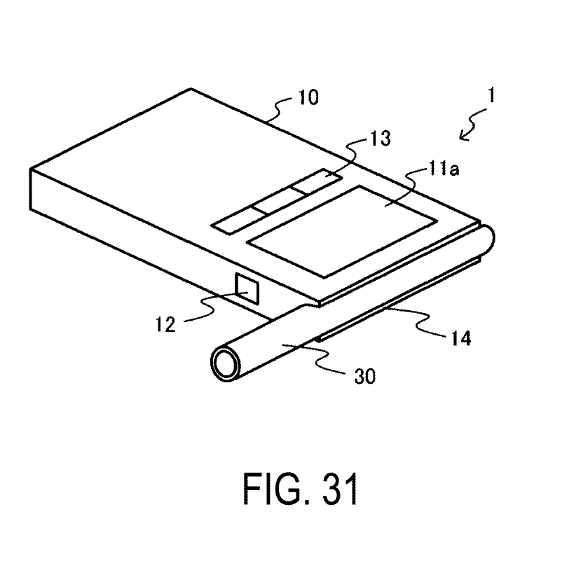

A breath component measuring assembly 1 is a device which measures an alcohol gas concentration which is a detection target gas component contained in breath of a subject, and, as illustrated in FIGS. 31, 32A to 32C, 33A to 33E, 34A to 34C and 35, has a breath component measuring device 10 which has a casing made of a hard material such as metal or resin and a mouthpiece 30 which is detachably attached to the breath component measuring device 10 and is made of hard material such as resin.

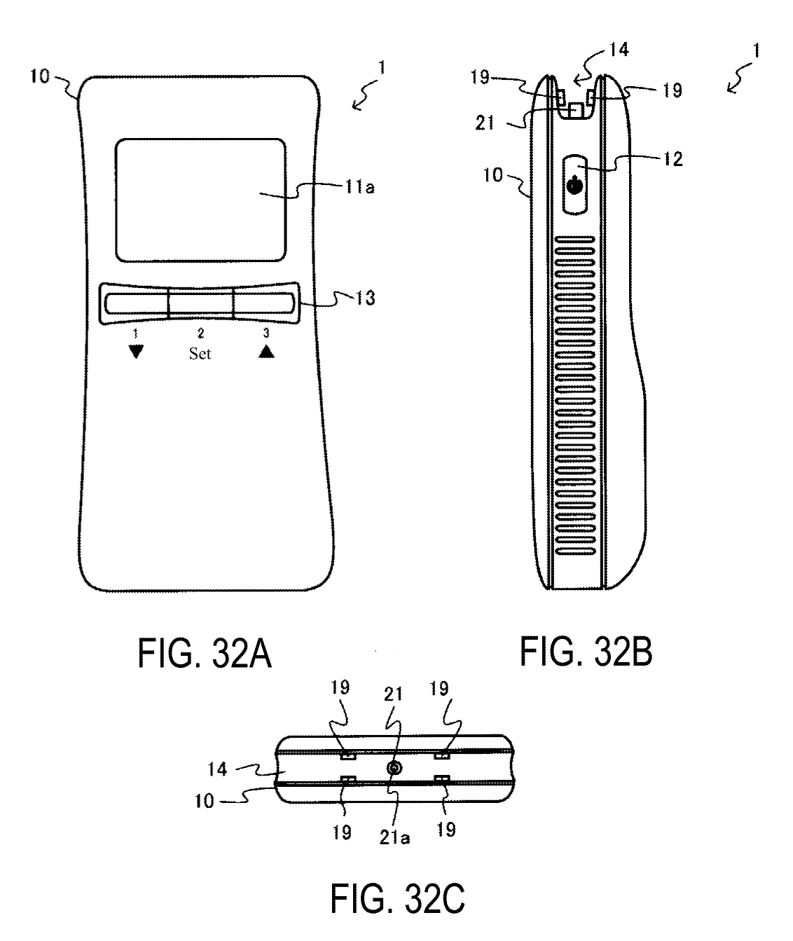

The breath component measuring device 10 of the breath component measuring assembly 1 is a device which acquires breath blown into the mouthpiece 30, and, as illustrated in FIGS. 32A and 32B, on an outer face of the breath component measuring device 10, provided are a power switch 12, an input interface 13 such as operation buttons, an attachment portion 14 to which the mouthpiece 30 in which the subject blows breath is attached, a projection 21 which is provided with a guide hole 21a which allows breath to flow thereinside, and a display unit 11a such as an LCD (Liquid Crystal Display) which displays, for example, a measurement result of a detection target gas component contained in breath based on a detection result of a gas sensor which measures a gas (alcohol) concentration in the breath.

In the present embodiment, the breath component measuring device 10 has, for example, a communication interface which is connected with a communication cable such as the information processing terminal 7, which is connected to an external device which will be described below and a power input terminal with which a power code which supplies power from an outside is connected although not illustrated. The breath component measuring device 10 can have a built-in battery such as a dry cell battery inside as a power source, and can be carried by a user.

As illustrated in FIGS. 32B and 32C, engaging pieces 19 which engage with the mouthpiece 30 are provided in side surfaces of the attachment portion 14, and the breath component measuring device 10 and the mouthpiece 30 are jointed through these engaging pieces 19. The mouthpiece 30 is detachably attached to the attachment portion 14.

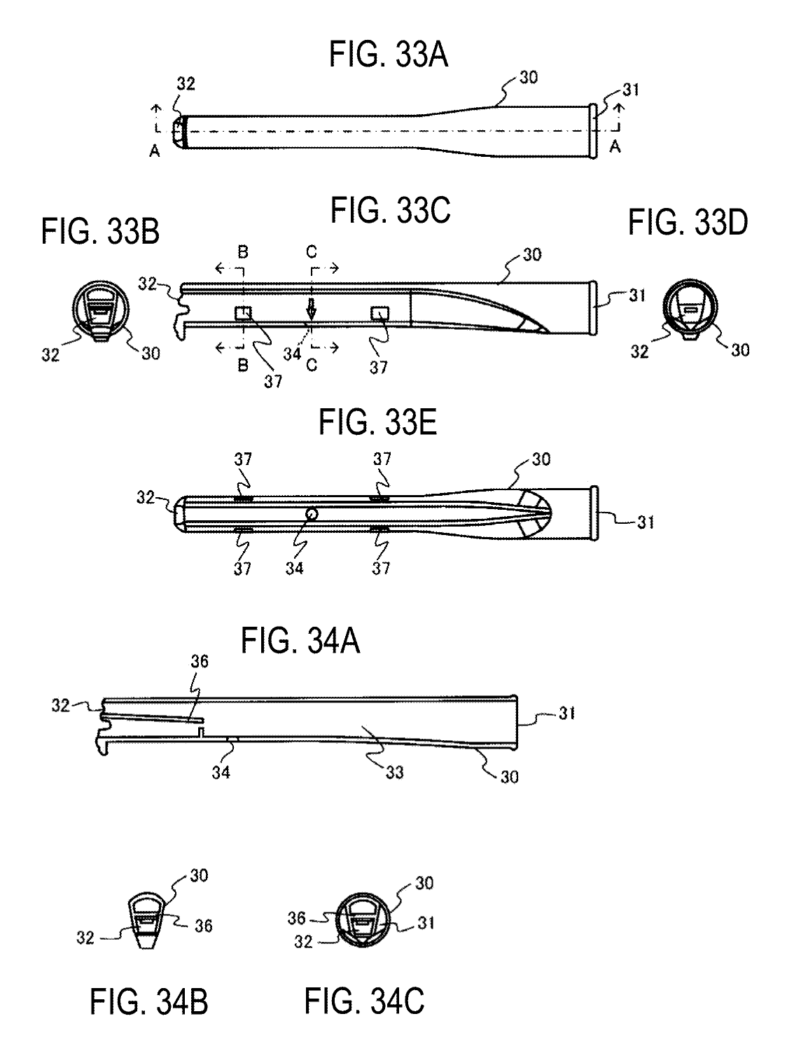

The mouthpiece 30 is a tubular blow unit in which the subject to be examined blows breath, and has a blow port 31 in which the subject blows breath, an outlet 32 which discharges breath and a breath flow path 33 which is formed between the blow port 31 and the outlet 32 and allows the breath blown from the blow port 31 to pass therethrough as illustrated in FIGS. 33A to 33E and 34A to 34C.

In the breath flow path 33, a penetration hole 34 to which the projection 21 of the breath component measuring device 10 is fitted when the mouthpiece 30 is attached to the breath component measuring device 10, and a partitioning wall 36 which is formed on an outlet 32 side and separates a flow of the breath passing through the breath flow path 33 and produces a sound are formed. The penetration hole 34 fits into the projection 21 to make the breath flow path 33 and a guide hole 21a formed in the projection 21 communicate with each other. When air flows therein from the blow port 31, the mouthpiece 30 produces a sound similar to a whistle. Even when the subject does not intentionally blow breath or blows breath in the mouthpiece only for a short period of time, a sound is not made by the mouthpiece 30 and such cheating immediately is found out. To measure an alcohol concentration in front of a person who monitors or supervises the subject, the subject cannot help but continually blowing breath in the mouthpiece 30.

Further, on an outer face of the mouthpiece 30, engaging holes 37 are formed so as to engage with the engaging pieces 19 when attached to the breath component measuring device 10. As illustrated in FIG. 35, when the mouthpiece 30 is attached to the attachment portion 14, the engaging pieces 19 of the breath component measuring device 10 are fitted to the engaging holes 37 of the mouthpiece 30, so that the mouthpiece 30 is stably held by the attachment portion 14.

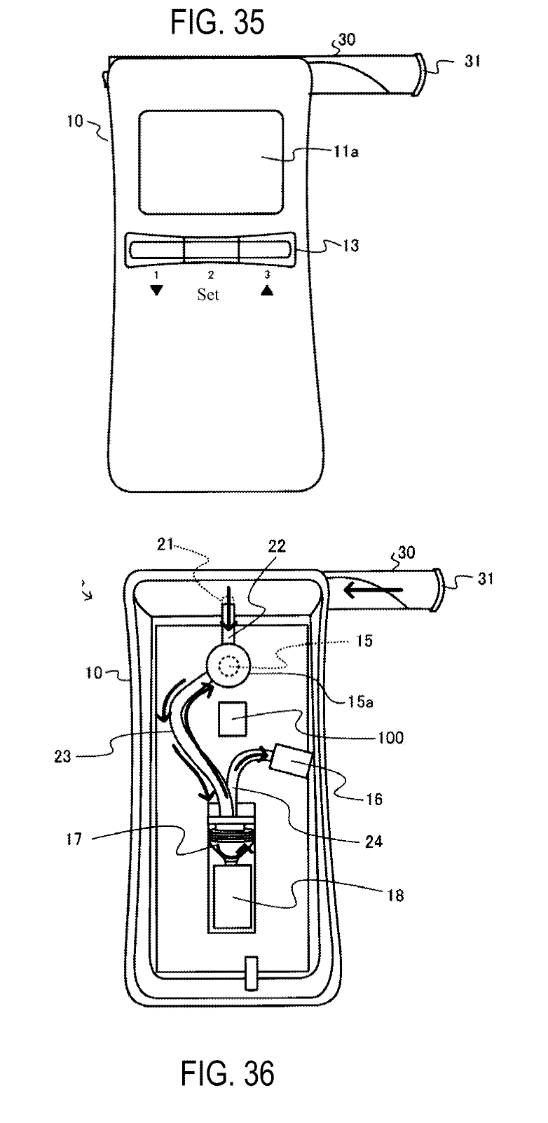

Next, an internal configuration of the breath component measuring device 10 will be described. As illustrated in FIG. 36, this breath component measuring device 10 has a gas sensor 15 which measures an alcohol concentration of breath blown in the breath component measuring device 10, a pressure sensor 16 which detects the pressure of breath blown in the breath component measuring device 10, an air barrel 17 which is a container which accumulates breath inside and can be expanded and contracted, and a solenoid 18 which contracts the air barrel 17 and guides the breath in the air barrel 17 to the gas sensor 15. Further, a flow path of breath in the breath component measuring device 10 includes a flow path 22 which guides breath blown from a guide hole 21a provided in the projection 21, to a gas sensor room 15a, a flow path 23 which connects the gas sensor room 15a and the air barrel 17 with each other and a flow path 24 which connects the air barrel 17 and the pressure sensor 16 with each other. The gas sensor room 15a is also part of the flow path, and the gas sensor 15 is arranged inside the gas sensor room 15a.

The air barrel 17 is made from flexible material and is a stretchable and airtight container which has a bellow shape, and the solenoid 18 is a driving mechanism which contracts this air barrel 17. Further, breath is blown in the air barrel 17 in a contracted state, and an air pressure of the breath expands the air barrel 17. Subsequently, the solenoid 18 contracts the air barrel 17, so that the breath accumulated therein is pushed back to the gas sensor room 15a.

The pressure sensor 16 is a detecting unit which detects a pressure of breath blown into the breath component measuring device 10. In the present embodiment, a semiconductor distortion gauge is formed on a surface of a diaphragm, and a semiconductor piezoresistance diffusing pressure sensor which converts a change of electric resistance resulting from a piezoresistance effect produced when the diaphragm deforms due to a force (pressure) from an outside, into an electrical signal is used as the pressure sensor 16.

The gas sensor 15 is a detecting unit which has a gas sensing body, is accommodated inside the gas sensor room 15a of the breath component measuring device 10 and detects gas in breath. The breath blown in the breath flow path is brought in contact with the gas sensing body. In the present embodiment, an electrochemical sensor which has a gas sensing body in which a current flows upon contact with alcohol, and detects the alcohol concentration in the gas according to a value of a flowing current is used as the gas sensor 15. For this electrochemical sensor, for example, Pt or Pt alloys are used as an anode and a cathode, and sulfuric acid (H₂SO₄) is used as electrolyte to measure a change in a current produced when alcohol molecules become oxidized by platinum catalyst.

The gas sensor 15 only needs to detect the alcohol concentration contained in breath, and can be selected from various alcohol sensors such as a semiconductor sensor which detects the alcohol concentration in gas based on electrical resistance which changes according to a reaction of oxygen adsorbed to a metal oxide and alcohol in the gas.

The gas sensor 15 deteriorates in some cases. Hence, the gas sensor 15 is preferably checked up on a regular basis. Although deterioration of the above electrochemical sensor in particular is accelerated depending on conditions, sensors of other types can also deteriorate.

In the present embodiment, the gas sensor 15 starts measuring the alcohol concentration when the pressure sensor 16 detects that breath is continuously blown in the breath component measuring device 10 for a predetermined period of time (for example, five seconds). In addition, start of detection of alcohol by the gas sensor 15 may be judged based on a condition other than a pressure of gas, and, for example, a sensor which detects a carbon dioxide gas concentration in gas in the breath component measuring device 10, an acoustic sensor which detects a sound produced in the mouthpiece 30 which is produced by breath of a subject or a temperature sensor (thermistor) which detects a temperature of gas in the breath component measuring device 10 is disposed in the breath component measuring device 10 in order to decide whether or not breath is continuously blown in the breath component measuring device 10 for a predetermined period of time.

Further, a control processing unit 100 such as a CPU is provided on a circuit substrate in the breath component measuring device 10. This control processing unit 100 is a computing module which is configured by hardware such as a processor like a CPU or a DSP (Digital Signal Processor), memory and other electronic circuits, software such as a program having a function of the hardware or a combination of these, virtually constructs various functional modules by adequately reading and executing the program, has each constructed functional module control measurement of the above breath component, and controls the entirety of the breath component measuring device 10 such as an input of a power switch 12 or an operation button and an output of a display unit 11a.

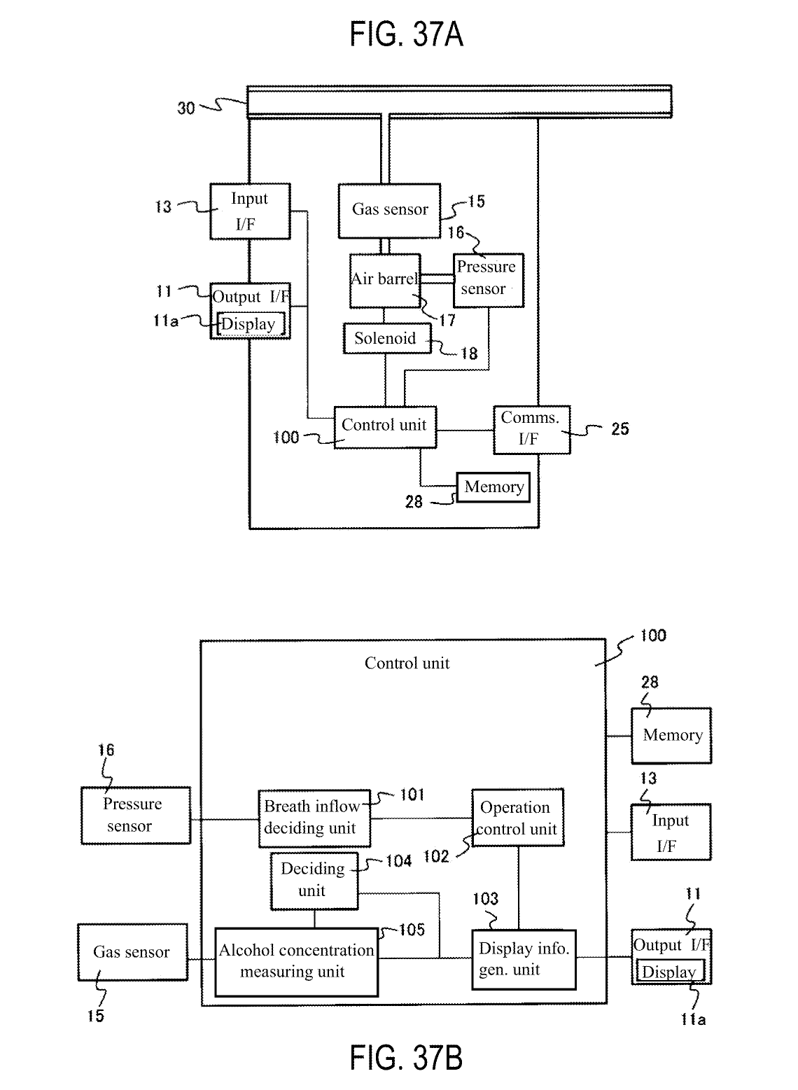

Next, a functional module of the breath component measuring device 10 will be described. FIG. 37A is a block diagram illustrating an entire configuration of the breath component measuring device 10 according to the present embodiment, and FIG. 37B is a block diagram illustrating an internal module of the control processing unit 100. In addition, a “module” which is used in the specification is configured by hardware such as an apparatus or a device, software which has a function thereof or a combination thereof, and means a functional unit which achieves a predetermined operation.

The breath component measuring device 10 has the input interface 13, an output interface 11, a communication interface 25, a memory 28 and the control processing unit 100.

The input interface 13 is a device such as an operation button, a touch panel or a jog dial which receives an input of a user operation. The output interface 11 is a device such as a display or a speaker which outputs an image or a sound. This output interface 11 in particular includes a display unit 11a which is an LCD which displays a measurement result of a detection target gas component contained in breath based on a detection result of the gas sensor 15 and information such as an operation guide.

The communication interface 25 is a communication interface with which the communication cable is connected, and transmits, for example, a measurement result to the information processing terminal connected through the communication cable. Instead of the communication cable, wireless communication such as Bluetooth (registered trademark) may be used.

The memory 28 is a memory device such as ROM or RAM which stores various items of data, and stores a threshold value with respect to the acoustic sensor 16 which determines whether or not the gas sensor 15 starts measurement, a threshold of an alcohol concentration value which determines whether or not driving is allowable and information such as a measurement result.

The control processing unit 100 is a computation processing device such as a CPU, and is a module which virtually configures each functional module by executing various programs on this control processing unit 100. In the present embodiment, the control processing unit 100 has an operation control unit 102, a display information generating unit 103, a deciding unit 104, a breath inflow deciding unit 101 and an alcohol concentration measuring unit 105.

The operation control unit 102 is a module which drives each device of the breath component measuring device 10, and drives, for example, the pressure sensor 16, the gas sensor 15 and the display unit 11a. Particularly, when breath is blown into the breath component measuring device 10 and the pressure sensor 16 continuously detects the pressure exceeding a predetermined threshold, the operation control unit 102 drives the solenoid 18 so as to push breath (gas) in the air barrel 17 back to a gas sensor room 15a. Further, the operation control unit 102 performs control to drive the gas sensor 15 and detect an alcohol concentration.

The breath inflow deciding unit 101 is a module which calculates a pressure value in the device based on the detection result of the pressure sensor 16, and decides start of an inflow of breath and continuation of breath. More specifically, when the subject blows breath and the pressure sensor 16 detects a predetermined pressure value, the breath inflow deciding unit 101 continuously detects a pressure value in a predetermined period of time while counting the predetermined period of time (for example, about five seconds). Further, when the pressure value detected by the breath inflow deciding unit 101 continuously exceeds the predetermined threshold, the operation control unit 102 drives the solenoid 18, and pushes breath in the air barrel 17 back to the gas sensor room 15a. This is because, when breath which exceeds a certain pressure continues, the amount of air required to measure the alcohol concentration is obtained in the air barrel 17. The subject needs to take a deep breath and exhale breath for a long period of time to obtain an alcohol measurement result.

The alcohol concentration measuring unit 105 is a module which measures the alcohol concentration which is the detection target gas component contained in breath based on a detection result of the gas sensor 15, and, more specifically, calculates the alcohol concentration in breath based on a detection result detected when air in the air barrel 17 is sent out to the gas sensor 15 by the operation control unit 102.

The deciding unit 104 is a module which decides whether or not driving is allowable, based on the alcohol concentration of the detection target gas component calculated by the alcohol concentration measuring unit 105. More specifically, the deciding unit 104 compares the calculated alcohol concentration in breath with the threshold stored in the memory 28, and decides that driving is allowable when the calculated alcohol concentration is the threshold or less.

The display information generating unit 103 is a module which displays various pieces of information on the display unit 11a, and, when, for example, the power switch 12 acquires an inputted signal, displays information about an operation guide such as an instruction to start blowing breath or a message to end blowing or displays, for example, a numerical value of the alcohol concentration detected by the gas sensor 15 or a decision result of the deciding unit 104 on the display unit 11a.

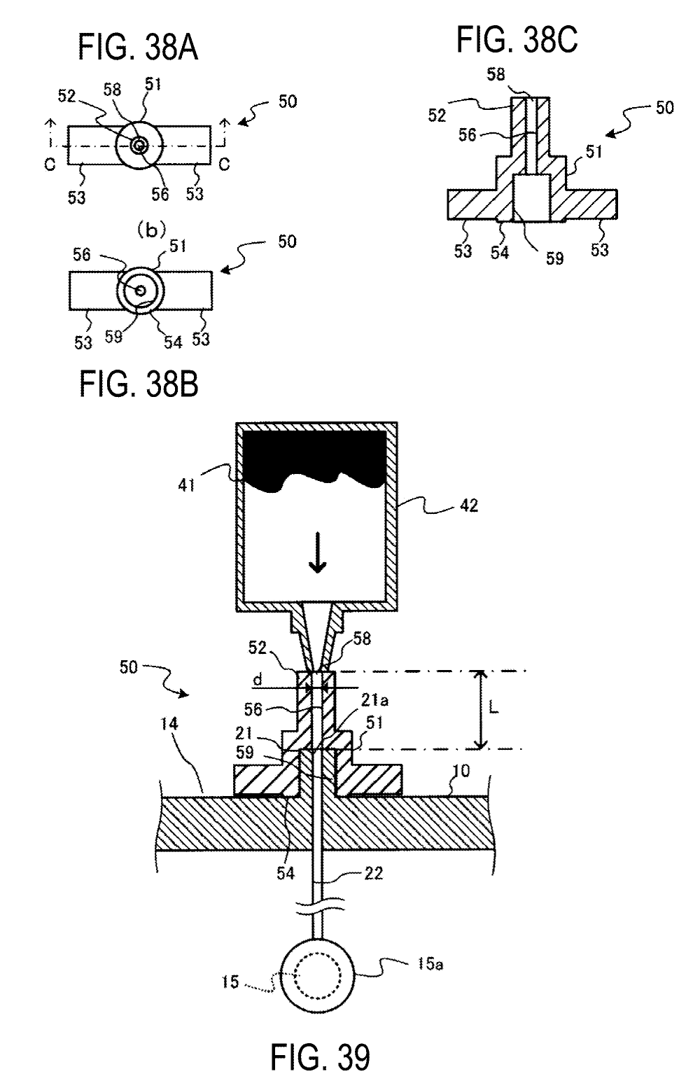

A test device for the above breath component measuring device 10 will be described. FIG. 38A is a top view illustrating the test device, FIG. 38B is a lower surface view of the test device and FIG. 38C is a cross-sectional view along C-C in FIG. 38A.

The test device 50 is made from a hard material such as resin. The test device 50 has an approximately tubular lower portion 51, a tubular upper portion 52 which is thinner than the lower portion 51 and two extended portions 53 which jut out sideward from the lower portion 51. An end surface of the lower portion 51 is a contact surface 54 which is brought in contact with the breath component measuring device 10. In the lower portion 51, a concave portion 59 which is opened in the contact surface 54 is formed. In the upper portion 52, a space 56 which has a circular cross section is formed. The concave portion 59 has a circular cross section, and a uniform diameter in a height direction of the test device 50 (the vertical direction in FIG. 38C). The space 56 also has a circular cross section, and has a uniform diameter (a diameter d in FIG. 39) in the height direction of the test device 50. The concave portion 59 and the space 56 are coaxial, and communicate to each other. The space 56 is opened in an upper end surface of the upper portion 52, and this opening is an inlet 58 which allows air containing alcohol to flow in the space 56. Hence, the inlet 58 is arranged the farthest apart from the contact surface 54 in the space 56.

The test device 50 is used in a state where the contact surface 54 is in contact with the breath component measuring device 10. FIG. 39 is a cross-sectional view of the test device 50 wherein a use state of the test device 50 and the breath component measuring device 10 is shown. As illustrated in FIG. 39, the contact surface 54 of the test device 50 is brought in contact with the breath component measuring device 10 by fitting the projection 21 of the breath component measuring device 10 in the concave portion 59 of the test device 50. The test device 50 and the breath component measuring device 10 form a sort of a breath component measuring assembly.

In this state, the space 56 of the test device 50 communicates with the guide hole 21a of the projection 21 of the breath component measuring device 10 and the flow path 22 in the breath component measuring device 10. In this state, when air containing alcohol is guided to the inlet 58 arranged the farthest apart from the contact surface 54, the air passes in the space 56 and passes through the guide hole 21a of the breath component measuring device 10, and reaches the gas sensor 15 in the breath component measuring device 10.

A method of guiding air containing alcohol to the inlet 58 of the test device 50 is assumed to include arranging gel containing alcohol (for example, gel for disinfecting hands) near the inlet 58 of the test device 50. For example, as illustrated in FIG. 39, the container 42 which contains gel 41 containing alcohol is turned upside down to fit an opening portion of the container 42 in the inlet 58 of the test device 50. The alcohol volatilizes from the gel 41, and the air containing the alcohol enters the inlet 58 of the test device 50 from the container 42. The gel containing the alcohol is low cost.

As long as the shape and the dimension of the test device 50 are adequately set, the alcohol concentration in the air guided in the breath component measuring device 10 appropriately attenuates, so that it is possible to adequately inspect the gas sensor 15. According to an investigation by the inventors, the space 56 (including the inlet 58) is preferably arranged on a straight line on which the guide hole 21a of the breath component measuring device 10 extends. Further, the space 56 (including the inlet 58) preferably has a diameter d from 0.05 mm to 2 mm. Furthermore, a length L from the inlet 58 of the test device 50 to the guide hole 21a of the breath component measuring device 10 is preferably 1 mm to 30 mm.

Still further, the diameter d of the space 56 (including the inlet 58) is preferably 1 mm to 1.5 mm. Moreover, the length L from the inlet 58 to the guide hole 21a of the breath component measuring device 10 is preferably 5 mm to 20 mm.

In the present embodiment, the breath component measuring device 10 has the projection 21 in which the guide hole 21a is formed, and a concave portion 59 to which the projection 21 is fitted and communicates with the space 56 is opened in the contact surface 54 of the test device 50. By adequately setting the length from the inlet 58 to the concave portion 59, it is possible to easily and adequately secure the length L from the inlet 58 to the guide hole 21a. More specifically, the length from the inlet 58 to the concave portion 59 is 1 mm to 30 mm, so that it is possible to easily secure 1 mm to 30 mm of the length L from the inlet 58 to the guide hole 21a. When the length from the inlet 58 to the concave portion 59 is 5 mm to 20 mm, it is possible to easily secure 5 mm to 20 mm of the length L from the inlet 58 to the guide hole 21a.

When the gas sensor 15 is checked up using the test device 50, a test person (checker) operates the input interface 13 of the breath component measuring device 10, and sets the breath component measuring device 10 to a test mode. In the test mode, the checker arranges the test device 50 as described above, and arranges the gel 41 containing alcohol near the inlet 58. The air containing alcohol enters the inlet 58 of the test device 50, passes in the space 56, further passes through the guide hole 21a of the breath component measuring device 10 and reaches the gas sensor 15 in the breath component measuring device 10.

The alcohol concentration measuring unit 105 (FIG. 37) analyzes an output of the gas sensor 15, and calculates the alcohol concentration in air. The display information generating unit 103 outputs the calculated alcohol concentration to the output interface 11. For example, the display information generating unit 103 displays the alcohol concentration on the display unit 11a. Based on the outputted measurement result, a checker may decide whether or not the breath component measuring device 10 normally operates. Alternatively, when the alcohol concentration calculated by the alcohol concentration measuring unit 105 is too low, the deciding unit 104 may generate warning information, and the display information generating unit 103 may output the warning information to the output interface 11.

The breath component measuring device 10 is set to a measurement mode by operating the input interface 13. In the measurement mode, the control processing unit 100 of the breath component measuring device 10 executes measurement of an alcohol concentration of breath of a subject and various operations related to this measurement. Various operations include pushing breath back to the gas sensor 15 using the pressure sensor 16, the solenoid 18 and the air barrel 17, and transmitting information to the information processing terminal 7 through the communication interface 25. However, in the test mode, these operations are not indispensable. Further, capturing an image of a subject using the digital camera 77 of the information processing terminal 7 is not indispensable, either.

When air of a very high alcohol concentration is supplied to the gas sensor 15 upon test of the gas sensor 15 in the test mode, the gas sensor 15 cannot normally measure the alcohol concentration when measuring the alcohol concentration of the subject using the breath component measuring device 10 next. Hence, when the alcohol concentration calculated by the display information generating unit 103 is very high (when, for example, the alcohol concentration exceeds 2 mg/l) upon test of the gas sensor 15 in the test mode, the control processing unit 100 of the breath component measuring device 10 may be disabled to measure the alcohol concentration of the subject for a certain period of time (for example, three minutes) and be enabled to measure the alcohol concentration of the subject after this period of time passes. In this case, the control processing unit 100 may have the display unit 11a of the breath component measuring device 10 display, for example, a guidance which provides an instruction to wait for measurement for three minutes. When the control processing unit 100 can communicate with the information processing terminal 7, the control processing unit 100 may transmit a signal for having the display unit 73a of the information processing terminal 7 display such a guidance, to the information processing terminal 7.

### Example 2

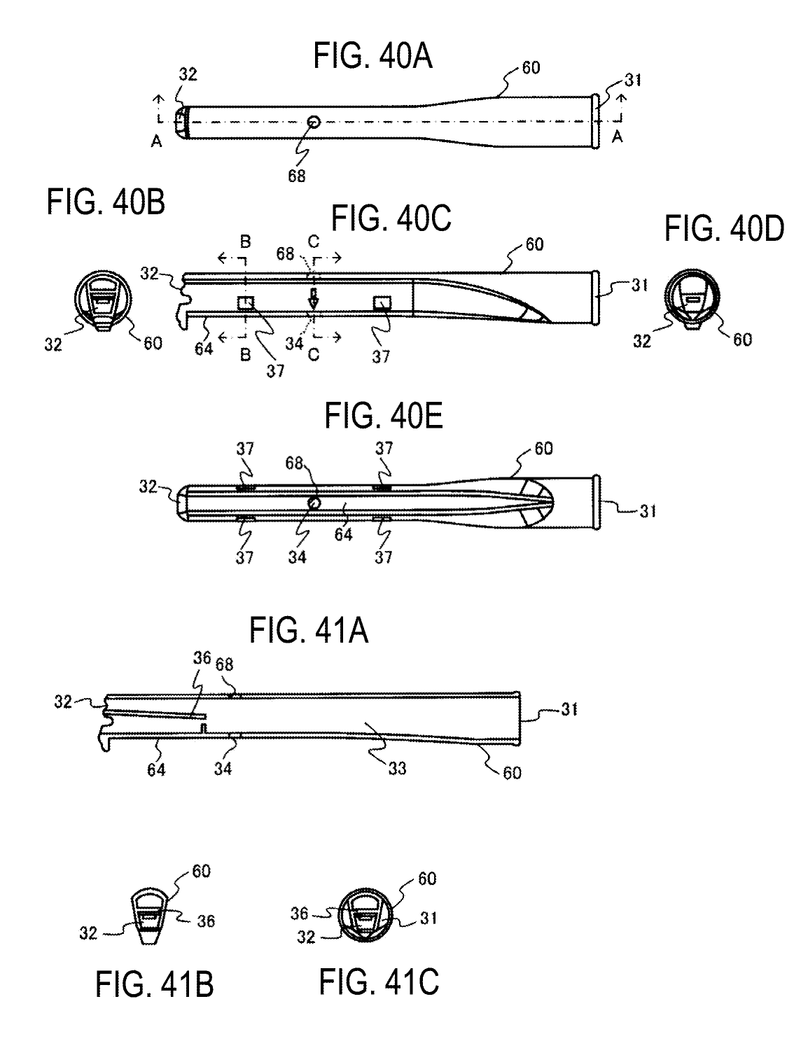

Next, a test device according to an EXAMPLE 2 of the FOURTH EMBODIMENT will be described. FIG. 40A is a top view illustrating a test device 60 according to the present embodiment, FIG. 40B is a front view of the test device 60, FIG. 40C is a side view of the test device 60, FIG. 40D is the back view of the test device 60 and FIG. 40E is a bottom view of the test device 60. Further, FIG. 41A is a cross-sectional view along A-A in FIG. 40A, FIG. 41B is a cross-sectional view along B-B in FIG. 40B, and FIG. 41C is a cross-sectional view along C-C in FIG. 40B.

As is clear upon comparison of FIGS. 33A to 33E, 34A to 34C, 40A to 40E and 41A to 41C, the test device 60 has the same shape as that of the mouthpiece 30 which a subject uses to blow breath in the breath component measuring device 10. In order to indicate the same components as the components used in the mouthpiece 30, the same reference numerals as those used in FIGS. 33A to 33E and 34A to 34C are used in FIGS. 40A to 40E and 41A to 41C. Similar to the mouthpiece 30, the test device 60 is formed using a hard material such as resin.

Meanwhile, unlike the mouthpiece 30, an inlet 68 which is a penetration hole is formed in an upper portion of the test device 60. The inlet 68 is coaxial with a penetration hole 34 formed in a lower portion of the test device 60. The penetration hole 34 and the inlet 68 have circular cross sections. The inlet 68 has a uniform diameter (a diameter d in FIG. 41) in the height direction of the test device 60.

Similar to the mouthpiece 30, the test device 60 is detachably attached to an attachment portion 14 of the breath component measuring device 10. In FIGS. 31, 32A to 32C, 33A to 33E, 34A to 34C and 35, the mouthpiece 30 can be replaced with the test device 60. The test device 60 and the breath component measuring device 10 form a sort of a breath component measuring assembly.

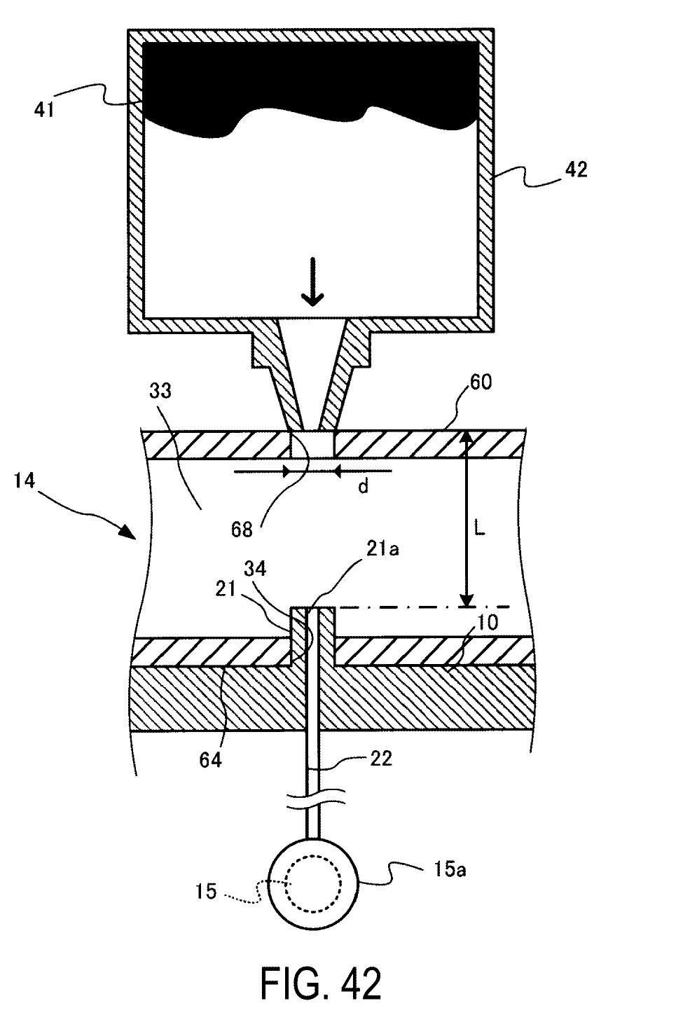

FIG. 42 is a cross-sectional view of the test device 60 which indicates a use state of the test device 60 and the breath component measuring device 10. The test device 60 is attached to the attachment portion 14 of the breath component measuring device 10. The test device 60 has at a lower portion a contact surface 64 which contacts the breath component measuring device 10. In this test device 60, a breath flow path 33 is used as a space which is used to supply air containing alcohol to a gas sensor 15 through a guide hole 21a of the breath component measuring device 10. The breath flow path (space) 33 is formed to communicate with the guide hole 21a of the breath component measuring device 10.

Similar to the mouthpiece 30, the test device 60 has the penetration hole (concave portion) 34. The operation hole 34 is opened in the contact surface 64, and communicates with the breath flow path 33. A projection 21 of the breath component measuring device 10 is fitted to the penetration hole 34.

The breath flow path (space) 33 has the inlet 68 which allows air containing alcohol to flow in the breath flow path 33. The inlet 68 is arranged in the breath flow path 33 the farthest apart from the contact surface 64, and is arranged on a straight line on which the guide hole 21a of the breath component measuring device 10 extends.

The test device 60 is used in a state where the contact surface 64 is in contact with the breath component measuring device 10. As illustrated in FIG. 42, the contact surface 64 of the test device 60 is placed in contact with the breath component measuring device 10 by fitting the projection 21 of the breath component measuring device 10 in the penetration hole 34 of the test device 60.

By this means, the breath flow path 33 of the test device 60 communicates with the guide hole 21a of the projection 21 of the breath component measuring device 10 and the flow path 22 in the breath component measuring device 10. In this state, when air containing alcohol is guided to the inlet 68 arranged the farthest apart from the contact surface 64, the air passes through the breath flow path 33 and passes through the guide hole 21a of the breath component measuring device 10, and reaches the gas sensor 15 in the breath component measuring device 10. Although the breath flow path (space) 33 extends in a direction (a horizontal direction in FIG. 42) vertical with respect to the straight line on which the guide hole 21a extends, the air containing alcohol can pass in the guide hole 21a.

A method of guiding air containing alcohol to the inlet 68 of the test device 60 is assumed to include arranging gel containing alcohol near the inlet 68 of the test device 60. For example, similar to the EXAMPLE 1, the container 42 which contains gel 41 containing alcohol is turned upside down to fit an opening portion of the container 42 to the inlet 68 of the test device 60. The alcohol volatilizes from the gel 41, and the air containing the alcohol enters the inlet 68 of the test device 60 from the container 42.

As long as the shape and the dimension of the test device 60 are adequately set, the alcohol concentration in the air guided in the breath component measuring device 10 appropriately attenuates, so that it is possible to adequately inspect the gas sensor 15. According to an investigation by the inventors, the inlet 68 is preferably arranged on a straight line on which the guide hole 21a of the breath component measuring device 10 extends. Further, the space 68 preferably has a diameter d from 0.05 mm to 2 mm. Furthermore, a length L from the inlet 68 of the test device 60 to the guide hole 21a of the breath component measuring device 10 is preferably 1 mm to 30 mm.

Still further, the diameter d of the inlet 68 is preferably 1 mm to 1.5 mm. Moreover, the length L from the inlet 68 to the guide hole 21a of the breath component measuring device 10 is preferably 5 mm to 20 mm.

In the present embodiment, the breath component measuring device 10 has the projection 21 in which the guide hole 21a is formed, and the penetration hole 34 to which the projection 21 is fitted and communicates with the breath flow path 33 is opened in the contact surface 64 of the test device 60. By adequately setting the length from the inlet 68 to the penetration hole 34, it is possible to easily and adequately secure the length L from the inlet 68 to the guide hole 21a.

When the gas sensor 15 is checked up using the test device 60, a test person (checker) operates an input interface 13 of the breath component measuring device 10, and sets the breath component measuring device 10 to a test mode. In the test mode, the checker arranges the test device 60 as described above, and arranges the gel 41 containing alcohol near the inlet 68. The air containing alcohol enters the inlet 68 of the test device 60, passes through the breath flow path 33, further passes through the guide hole 21a of the breath component measuring device 10 and reaches the gas sensor 15 in the breath component measuring device 10. Consequently, it is possible to decide whether or not the breath component measuring device 10 normally operates similar to the FIRST EMBODIMENT.

Similar to the mouthpiece 30, this test device 60 can be used to supply breath of a subject to the breath component measuring device 10 and measure an alcohol concentration of breath in a state where the test device 60 is attached to the breath component measuring device 10.

### Example 3

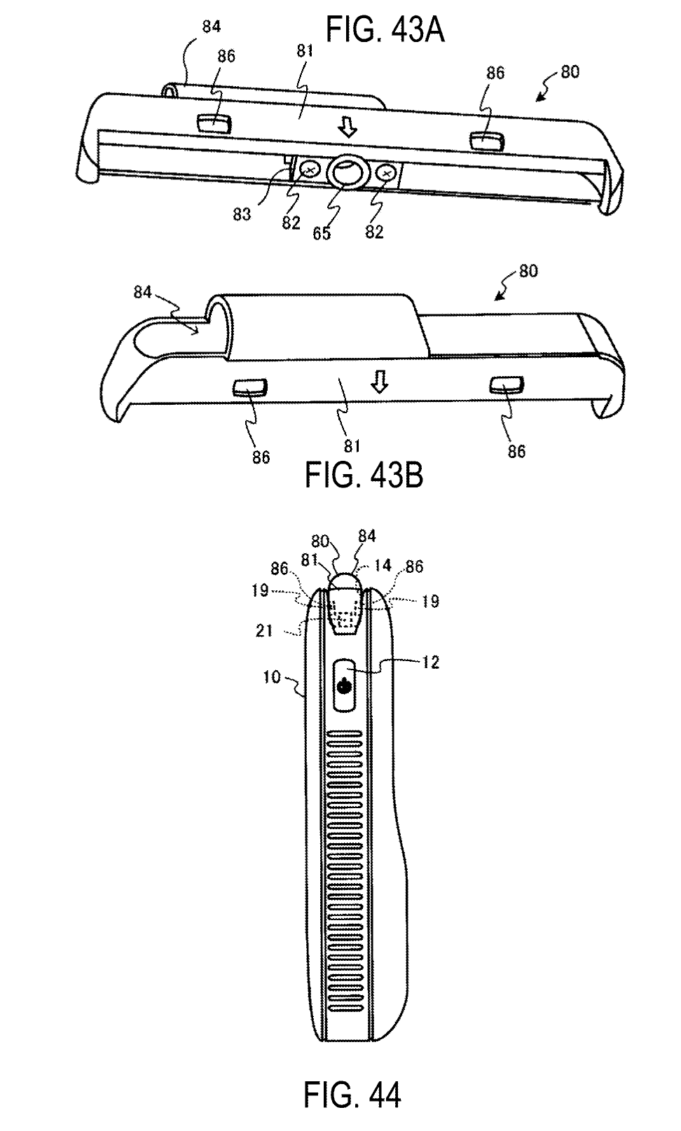

FIG. 43A is a perspective view illustrating a test attachment 80 according to an EXAMPLE 3 of the FOURTH EMBODIMENT, which is diagonally viewed from a lower side thereof, and FIG. 43B is a perspective view illustrating the test attachment 80, which is diagonally viewed from an upper side thereof. As illustrated in FIG. 44, the test attachment 80 is detachably attached to an attachment portion 14 of a breath component measuring device 10.

This test attachment 80 is attached to the breath component measuring device 10 to test a gas sensor 15 of the breath component measuring device 10. The test device 80 and the breath component measuring device 10 form a sort of a breath component measuring assembly.

The test attachment 80 is formed using a hard material such as resin. As illustrated in FIG. 43A, the test attachment 80 has a long holding piece 81 and a test device 83 which is fixed to the holding piece 81 by screws 82. As illustrated in FIGS. 43A and 43B, in the holding piece 81, engaging holes 86 are formed which engage with engaging pieces 19 when the holding piece 81 is attached to the breath component measuring device 10.

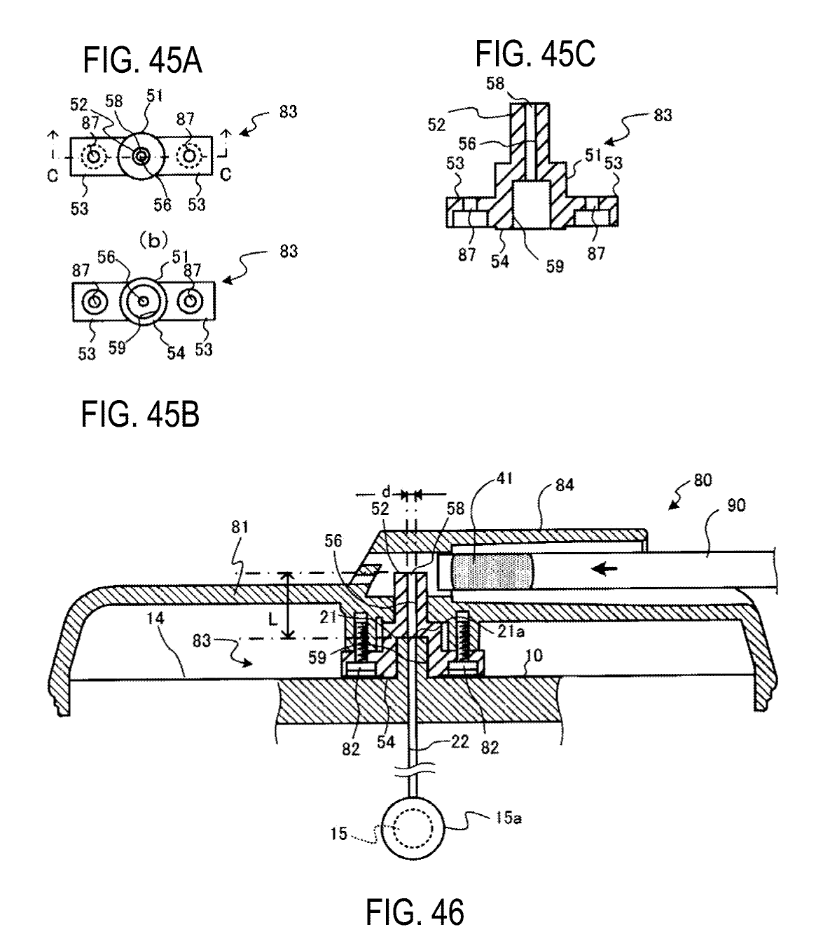

FIG. 45A is a top view illustrating a test device 83, FIG. 45B is a lower surface view of the test device 83, and FIG. 45C is a cross-sectional view along C-C in FIG. 45A. This test device 83 has substantially the same shape as that of a test device 50 according to an EXAMPLE 1 (see FIGS. 38A to 38C). In order to indicate the same components as the components used in the test device 50, the same reference numerals as those used in FIGS. 38A to 38C are used in FIGS. 45A to 45C. Meanwhile, unlike the test device 50, penetration holes 87 are formed in respective extended portions 53 of the test device 83.

FIG. 46 is a cross-sectional view of the test attachment 80, wherein a use state of the test attachment 80 and the breath component measuring device 10 is shown. The holding piece 81 of the test attachment 80 is detachably attached to the attachment portion 14 of a breath component measuring device 10. The holding piece 81 has a cavity portion in which the test device 83 is arranged. Inside this cavity portion, bosses in which the screws 82 are screwed, and the screws 82 penetrate the penetration holes 87 of the test device 83 and are fixed to the bosses. Thus, the test device 83 is fixed to the holding piece 81 by the screws 82.

In the holding piece 81, a straw holder 84 is formed. It is possible to insert a straw 90 in an internal space of the straw holder 84. An upper portion 52 of the test device 83 which is attached to the holding piece 81 by the screws 82 projects into the internal space of the straw holder 84. Hence, the internal space of the straw holder 84 communicates with the space 56 of the test device 83.

When the holding piece 81 is attached to the attachment portion 14, the projection 21 of the breath component measuring device 10 just fits in the concave portion 59 of the test device 83, and the contact surface 54 of the test device 83 contacts the breath component measuring device 10. In this state, the space 56 of the test device 83 communicates with the guide hole 21a of the projection 21 of the breath component measuring device 10 and the flow path 22 in the breath component measuring device 10. In this state, the internal space of the straw holder 84 also communicates with the guide hole 21a of the projection 21 of the breath component measuring device 10 and the flow path 22 in the breath component measuring device 10.

When the gas sensor 15 is checked up using the test attachment 80, a test person (checker) operates the input interface 13 of the breath component measuring device 10, and sets the breath component measuring device 10 to a test mode. In the test mode, the checker arranges the test attachment 80 as described above, and arranges the gel 41 containing alcohol near the inlet 58 of the test device 83. More specifically, the checker attaches the test attachment 80 to the attachment portion 14 of the breath component measuring device 10, sucks the gel 41 containing alcohol using the straw 90, inserts the straw 90 in the straw holder 84 and blows the gel 41 containing alcohol in the straw 90 by means of breath. By this means, air containing alcohol circulates in the test device 83, and flows in a flow path in the breath component measuring device 10 from the guide hole 21a. That is, the air containing alcohol enters the inlet 58 of the test device 83, passes in the space 56, further passes through the guide hole 21a of the breath component measuring device 10 and reaches the gas sensor 15 in the breath component measuring device 10.

Consequently, it is possible to decide whether or not the breath component measuring device 10 normally operates similar to the FIRST EMBODIMENT. This test device 83 has substantially the same shape as that of a test device 50 according to FIRST EMBODIMENT. consequently, as described above in relation to the test device 50 according to the FIRST EMBODIMENT, as long as the shape and the dimension of the test device 83 (the diameter d of the space 56 and the length L from the inlet 58 to the guide hole 21a in particular) are adequately set, the alcohol concentration in the air guided in the breath component measuring device 10 appropriately attenuates, so that it is possible to adequately inspect the gas sensor 15. The holding piece 81 of the test attachment 80 plays a role of facilitating an arrangement of the test device 83 to an adequate position.

The preceding description has been presented only to illustrate and describe exemplary embodiments of the present mouthpiece of breath component measuring device, breath component measuring assembly, breath component measuring device, and breath component measuring system. It is not intended to be exhaustive or to limit the invention to any precise form disclosed. It will be understood by those skilled in the art that various changes may be made and equivalents may be substituted for elements thereof without departing from the scope of the invention. In addition, many modifications may be made to adapt a particular situation or material to the teachings of the invention without departing from the essential scope. Therefore, it is intended that the invention not be limited to the particular embodiment disclosed as the best mode contemplated for carrying out this invention, but that the invention will include all embodiments falling within the scope of the claims. The invention may be practiced otherwise than is specifically explained and illustrated without departing from its spirit or scope.

What is claimed is:

1. A mouthpiece which is attached to a breath component measuring device which measures an alcohol concentration of breath of a subject, the mouthpiece comprising:
a blow port into which the breath of the subject is blown;
a breath flow path which allows the breath blown into the blow port to flow in the breath component measuring device; and
a sound producing portion which produces a sound by means of a flow of the breath.

2. A breath component measuring assembly comprising:
the mouthpiece according to claim 1 and a breath component measuring device to which the mouthpiece is attached, wherein:
the breath component measuring device comprises:
a flow path in which the breath flows from the breath flow path of the mouthpiece;
a gas sensor which measures an alcohol concentration of the breath in the flow path;
an acoustic measuring device which measures an acoustic level of a sound produced by the mouthpiece; and
a control unit which starts measuring the alcohol concentration using the gas sensor when the acoustic level measured by the acoustic measuring device continuously exceeds a threshold for a certain period of time.

3. A breath component measuring device to which the mouthpiece according to claim 1 is to be attached to form a breath component measuring assembly together with the mouthpiece, the breath component measuring device comprising:
a flow path in which the breath flows from the breath flow path of the mouthpiece;
a gas sensor which measures an alcohol concentration of the breath in the flow path;
an acoustic measuring device which measures an acoustic level of a sound produced by the mouthpiece; and
a control unit which starts measuring the alcohol concentration using the gas sensor when the acoustic level measured by the acoustic measuring device continuously exceeds a threshold for a certain period of time.

4. A breath component measuring system comprising:
a breath component measuring assembly which comprises the mouthpiece according to claim 1 and a breath component measuring device to which the mouthpiece is attached; and
an information processing device which can communicate with the breath component measuring device, wherein:
the breath component measuring device comprises:
a flow path in which breath flows from the breath flow path of the mouthpiece;
a gas sensor which measures an alcohol concentration of the breath in the flow path; and
a control unit which starts measuring an alcohol concentration using the gas sensor;
the information processing device comprises an acoustic measuring device which measures an acoustic level of a sound produced by the mouthpiece; and the control unit of the breath component measuring device starts measuring the alcohol concentration using the gas sensor when the acoustic level measured by the acoustic measuring device continuously exceeds a threshold for a certain period of time.
